(19) United States
(12) Patent Application Publication
(10) Pub. No.: US 2026/0263471 A1
(43) Pub. Date: Sep. 10, 2026

(54) Methods of Treating Myelofibrosis

(71) Applicant: Kartos Therapeutics, Inc., Redwood City, CA (US)

(72) Inventors: Wayne Rothbaum, Delray Beach, FL (US); Srdan Verstovsek, Bellaire, TX (US); Richard Levy, New York, NY (US)

(73) Assignee: Kartos Therapeutics, Inc., Redwood City, CA (US)

(21) Appl. No.: 19/662,780

(22) Filed: Apr. 29, 2026

Related U.S. Application Data
(63) Continuation of application No. PCT/US2025/016620, filed on Feb. 20, 2025.
(60) Provisional application No. 63/555,643, filed on Feb. 20, 2024.
(60) Provisional application No. 63/727,025, filed on Dec. 2, 2024.
(60) Provisional application No. 63/726,889, filed on Dec. 2, 2024.

(51) Int. Cl. A61K 31/519; A61K 31/451; A61P 35/00

(52) U.S. Cl. CPC A61K 31/519; A61K 31/451; A61P 35/00

(57) ABSTRACT

Methods of treating myelofibrosis using a Mouse double minute 2 homolog (MDM2) inhibitor and a JAK inhibitor as a combination therapy for patients having a suboptimal response to the JAK inhibitor monotherapy.

### FIELD OF THE INVENTION

Methods of treating myelofibrosis using a Mouse double minute 2 homolog (MDM2) inhibitor and a JAK inhibitor.

### BACKGROUND OF THE INVENTION

Myelofibrosis (MF, including primary MF (PMF), post-polycythemia vera MF (post-PV MF), or post-essential thrombocythemia MF (post-ET MF) is a clonal myeloproliferative neoplasm (MPN), characterized by progressive bone marrow fibrosis and subsequent ineffective erythropoiesis, dysplastic megakaryocyte hyperplasia, and extramedullary hematopoiesis. The typical clinical presentation includes marked splenomegaly, progressive anemia, and constitutional symptoms. Additional risk factors that worsen the prognosis include age of greater than 65 years, hemoglobin of less than 10 g/dL, white blood cell (WBC) of greater than 25×109/L, and blood blasts of greater than or equal to 1%. The median survival at diagnosis in patients with high-risk disease is approximately 2 years (Cervantes 2009).

MF is a clonal stem-cell disease characterized by molecular alterations in driver genes (e.g., JAK2, MPL, CALR), and cytogenetic (e.g., 13q-, 20q-) markers (Pikman 2006; Hussein 2009). The JAK2 V617F mutation, which leads to overactive signaling from cytokine and growth factor receptors in hematopoietic progenitor cells, has been identified in over 95% of patients with PV and in approximately 50% of patients with ET and PMF.

Ruxolitinib, a Janus kinase inhibitor, is approved in the United States and the European Union for the treatment of patients with intermediate, or high-risk MF, including PMF, post-PV MF, and post-ET MF (Jakafi Prescribing Information). Treatment with ruxolitinib monotherapy results in spleen volume reduction (SVR) (e.g., SVR of greater than or equal to 35%) in approximately 28% to 42% of MF patients, and provides improvement in constitutional symptoms (e.g., total symptom score (TSS) reduction of greater than or equal to 50%) in approximately 46% of MF patients (Harrison 2012; Verstovsek 2012). However, only approximately a quarter (e.g., 20-25%) of responding patients achieve both SVR greater than or equal to 35% and TSS greater than or equal to 50%. On the other hand, a small proportion of patients do not achieve any clinically meaningful response after treatment with ruxolitinib monotherapy (e.g., the primary refractory population). Benefit in SVR and TSS reduction from ruxolitinib treatment is reportedly achieved within 12 weeks after initiating treatment, and then plateau, with minimal additional response benefit (Verstovsek 2012). Ruxolitinib does not significantly affect bone marrow fibrosis, or variant allele frequency (VAF) of driver mutations (e.g., JAK2 or CALR) within the first 1-2 years of treatment, suggesting a lack of effect on the underlying disease pathology (Verstovsek 2012). Lastly, roughly 50% of patients with MF discontinue ruxolitinib treatment within 3 years (Harrison 2016; Al-Ali 2017; Verstovsek 2017) with lack of achieving significant SVR, or loss of SVR accounting for approximately 35% of all patient discontinuations (Palandri 2020).

### SUMMARY OF THE INVENTION

In one aspect, the present invention relates to a method for treating myelofibrosis in a human subject who has been previously treated for myelofibrosis with ruxolitinib monotherapy and had a suboptimal response to said previous treatment with ruxolitinib monotherapy comprising concurrent administration to the human of a therapeutically effective amount of navtemadlin or a pharmaceutically acceptable salt thereof and ruxolitinib.

In some embodiments, navtemadlin or a pharmaceutically acceptable salt thereof is administered as an adjunct therapy in a human suffering from myelofibrosis.

In some embodiments, the spleen volume reduction (SVR) in the human after the ruxolitinib monotherapy is <35% compared to the spleen volume prior to the ruxolitinib monotherapy; and the total symptom score (TSS) of the human after the ruxolitinib monotherapy is less than 50% compared to a TSS prior to the ruxolitinib monotherapy.

In some embodiments, the spleen volume reduction (SVR) in the human after the previous treatment with ruxolitinib monotherapy is >zero but <35% compared to the spleen volume prior to the previous treatment with ruxolitinib monotherapy; and the total symptom score (TSS) of the human after the previous treatment with ruxolitinib monotherapy is >zero but less than 50% compared to a TSS prior to the previous treatment with ruxolitinib monotherapy.

In some embodiments, the spleen volume reduction (SVR) in the human after the previous treatment with ruxolitinib monotherapy is >zero but <20% compared to the spleen volume prior to the previous treatment with ruxolitinib monotherapy; and the total symptom score (TSS) of the human after the previous treatment with ruxolitinib monotherapy is >zero but less than 30% compared to a TSS prior to the previous treatment with ruxolitinib monotherapy.

In some embodiments, the spleen volume reduction (SVR) in the human after the previous treatment with ruxolitinib monotherapy is ≥20% but <35% compared to the spleen volume prior to the previous treatment with ruxolitinib monotherapy; and the total symptom score (TSS) of the human after the previous treatment with ruxolitinib monotherapy is ≥30% but <50% compared to a TSS prior to the previous treatment with ruxolitinib monotherapy.

In some embodiments, the ruxolitinib monotherapy is about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, or about 52 weeks. In some embodiments, the ruxolitinib monotherapy is about one month, about two months, about three months, about four months, about five months, about six months, about seven months, about eight months, about nine months, about ten months, about eleven months, or about twelve months. In some embodiments, the ruxolitinib monotherapy is about one year, about two years, about three years, about four years, or about five years. In some embodiments, the ruxolitinib monotherapy is at least 12 weeks. In some embodiments, the ruxolitinib monotherapy is at least 18 weeks.

In some embodiments, the concurrent administration is at least 12 weeks, at least 16 weeks, at least 18 weeks, at least 24 weeks, at least 28 weeks, at least 32 weeks, at least 36 weeks, at least 40 weeks, at least 44 weeks, at least 48 weeks, or at least 52 weeks. In some embodiments, navtemadlin or a pharmaceutically acceptable salt thereof is administered at a dose of 240 mg daily.

In some embodiments, navtemadlin or a pharmaceutically acceptable salt thereof is administered on days 1-7 of a 28-day treatment cycle; wherein navtemadlin or a pharmaceutically acceptable salt thereof is not administered on days 8-28 of the 28-day treatment cycle.

In some embodiments, navtemadlin or a pharmaceutically acceptable salt thereof is administered for one or more treatment cycles. In some embodiments, navtemadlin or a pharmaceutically acceptable salt thereof is administered for one, two, three, four, five, six, seven, eight, nine or ten treatment cycles, wherein each treatment cycle is a 28-day treatment cycle, wherein navtemadlin or a pharmaceutically acceptable salt thereof is administered on days 1-7 of the 28-day treatment cycle; and is not administered on days 8-28 of the 28-day treatment cycle.

In some embodiments, the spleen volume reduction (SVR) in the human after the one or more treatment cycles of navtemadlin adjunct therapy is at least 25%, 30%, 35%, 40%, 45%, 50%, 55%, 60%, 65%, 70%, 75%, 80%, 85%, 90%, or 95% compared to the spleen volume prior to the one or more treatment cycles.

In some embodiments, the spleen volume reduction (SVR) in the human after the one or more treatment cycles of navtemadlin adjunct therapy is at least 35%, 40%, 45%, 50%, 55%, 60%, 65%, 70%, 75%, 80%, 85%, 90%, or 95% compared to the spleen volume prior to the previous treatment with ruxolitinib monotherapy.

In some embodiments, the TSS in the human after the one or more treatment cycles of navtemadlin adjunct therapy is reduced by at least 30%, 40%, 45%, 50%, 55%, 60%, 65%, 70%, 75%, 80%, 85%, 90%, or 95% compared to a TSS prior to the one or more treatment cycles.

In some embodiments, the TSS of the human after the one or more treatment cycles of navtemadlin adjunct therapy is reduced by at least 50%, 55%, 60%, 65%, 70%, 75%, 80%, 85%, 90%, or 95% compared to a TSS prior to the ruxolitinib monotherapy.

In some embodiments, the SVR in the human after the one or more treatment cycles of navtemadlin adjunct therapy is at least 25% compared to the spleen volume prior to the one or more treatment cycles and at least 35% compared to the spleen volume prior to the ruxolitinib monotherapy.

In some embodiments, the TSS of the human after the one or more treatment cycles of navtemadlin adjunct therapy is reduced by at least 30% compared to a TSS prior to the one or more treatment cycles and at least 50% compared to the TSS prior to the ruxolitinib monotherapy.

In one preferred embodiment, the SVR in the human after the one or more treatment cycles of navtemadlin adjunct therapy is at least 25% compared to the spleen volume prior to the one or more treatment cycles and at least 35% compared to the spleen volume prior to the ruxolitinib monotherapy; wherein the TSS of the human after the one or more treatment cycles of navtemadlin adjunct therapy is reduced by at least 30% compared to a TSS prior to the one or more treatment cycles of navtemadlin adjunct therapy and at least 50% compared to the TSS prior to the ruxolitinib monotherapy.

In some embodiments, ruxolitinib is administered at a dose of 5 mg, 10 mg, 15 mg, 20 mg, or 25 mg twice daily.

In some embodiments, the human has a JAK2V617F mutation.

In some embodiments, the human does not have TP53 mutation.

In some embodiments, the human is JAK inhibitor-naïve prior to ruxolitinib monotherapy.

### BRIEF DESCRIPTION OF THE DRAWINGS

The foregoing summary, as well as the following detailed description of the invention, will be better understood when read in conjunction with the appended drawings.

FIG. 1 illustrates an example schematic for a randomized, double-blind study evaluating safety and efficacy of navtemadlin plus ruxolitinib versus placebo plus ruxolitinib in myelofibrosis patients who have a suboptimal response to ruxolitinib, in accordance with an embodiment of the present disclosure. Each treatment cycle is 28 days. 1: Stable Rux Dose is defined as ≥5 mg BID that does not require a treatment hold or dose adjustment in the 8 weeks prior to add-on treatment with navtemadlin. 2: Ruxolitinib will continue to be administered at the Stable Rux Dose. 3. The run-in period baseline assessment occurs prior to the start of ruxolitinib monotherapy dosing. 4. After ≥18 weeks but <25 weeks of treatment, response to ruxolitinib monotherapy will be assessed in subjects that have had a stable dose of ruxolitinib for ≥6 consecutive weeks (e.g., a dose ≥5 mg BID that did not require a treatment hold or dose adjustment). A stable dose of ruxolitinib ≥8 consecutive weeks is set as a baseline for randomization. Response assessment during the run-in period is allowed earlier in some implementations, after a stable dose of ≥6 consecutive weeks. Abbreviations: JAK, Janus kinase; MF, myelofibrosis; QD, daily; SVR, spleen volume reduction; TSS, total symptom score; EOS, end-of-study.

FIG. 2 illustrates example plots showing that navtemadlin combined with ruxolitinib enhances apoptosis in MF patient-derived progenitor cells, in accordance with an embodiment of the present disclosure. Abbreviation: MFI, median fluorescent intensity; NVTM, navtemadlin; Rux, ruxolitinib. Live cells were defined as CD45+mid, SSClow, CD14-, cPARP.

FIG. 3 illustrates example plots showing that navtemadlin combined with ruxolitinib reduces pro-survival MCL-1 levels, in accordance with an embodiment of the present disclosure. Abbreviation: MFI, median fluorescent intensity; NVTM, navtemadlin; Rux, ruxolitinib.

FIGS. 4A, 4B, and 4C collectively illustrate that cytotoxicity of navtemadlin as monotherapy or as add-on therapy to ruxolitinib is correlated with spleen volume reduction, in accordance with an embodiment of the present disclosure. FIG. 4A: Navtemadlin-Induced Reduction in CD34⁺ Cell Count is Correlated with SVR; FIGS. 4B and 4C: Change in CD34+ Cell Count. Abbreviations: C1D1, Cycle 1 Day 1; SVR-35, spleen volume reduction >35%. Data from 1 patient with a cell concentration of 0 cells/μL at Week 24 is not visible in FIG. 4C.

FIG. 5 provides example plots illustrating that navtemadlin-induced reduction in driver gene variant allele frequency is correlated with PFS and OS, in accordance with an embodiment of the present disclosure.

FIG. 6 provides example plots illustrating that navtemadlin-induced reduction in CD34⁺ cell count is correlated with PFS and OS, in accordance with an embodiment of the present disclosure.

FIG. 7 illustrates spleen volume reduction after addition of navtemadlin to a stable dose of ruxolitinib, in accordance with an embodiment of the present disclosure. Evaluable subjects with spleen volume assessments at baseline and Week 24 are shown. Six subjects discontinued treatment prior to Week 24.

FIG. 8 illustrates total symptom score reduction after addition of navtemadlin to a stable dose of ruxolitinib, in accordance with an embodiment of the present disclosure.

FIG. 9 illustrates reduction in driver variant allele frequency and improvement in bone marrow fibrosis after addition of navtemadlin to ruxolitinib, in accordance with an embodiment of the present disclosure. Abbreviations: BM, bone marrow; CALR, calreticulin; JAK2, Janus kinase; MF, myelofibrosis; VAF, variant allele frequency.

FIG. 10 provides multi-component spleen response endpoints based on spleen volume reduction, in accordance with an embodiment of the present disclosure. In FIG. 12, patients who achieve an SVR35 from the pre-ruxolitinib baseline are defined as responders (despite achieving a SVR25 from pre-randomization baseline), thereby attributing clinically meaningful benefit according to established threshold. Abbreviation: SVR, spleen volume reduction, SVR35, spleen volume reduction ≥35%, SVR25, spleen volume reduction ≥25%. The pre-ruxolitinib baseline is also referred as prior to the ruxolitinib monotherapy baseline. The pre-randomization baseline is also referred as after the ruxolitinib monotherapy baseline.

FIG. 11 provides multi-component spleen response endpoints based on TSS, in accordance with an embodiment of the present disclosure. In FIG. 13, patients who achieve an TSS50 from the pre-ruxolitinib baseline are defined as responders (despite achieving a TSS30 from pre-randomization baseline), thereby attributing clinically meaningful benefit according to established threshold. Abbreviation: TSS, total symptom score, TSS30, ≥30% TSS reduction, TSS50, ≥50% TSS reduction.

FIG. 12 illustrates suboptimal spleen and symptom responders from pooled phase 3 ruxolitinib data, in accordance with an embodiment of the present disclosure. Pooled extractable data from published phase 3 JAKi-naïve MF studies for patients treated with ruxolitinib monotherapy (±placebo) SVR (N=465): COMFORT-1, SIMPLIFY-1, and TRANSFORM-1(Verstovsek 2012; Mesa 2017; Pemmaraju 2023). TSS (N=528): COMFORT-1, SIMPLIFY-1, and MANIFEST-2 (Verstovsek 2012; Mesa 2017; Rampal 2023). Week-24 data used as a proxy to Week-18 suboptimal response assessment. Abbreviations: SVR, spleen volume reduction, TSS total symptom score.

FIG. 13 illustrates suboptimal spleen responders based on a SVR25 threshold, in accordance with an embodiment of the present disclosure. Utilizing the pooled dataset, it was determined that among the JAKi-naive MF patients with a suboptimal response to ruxolitinib the mean SVR was 18%. Studies Pooled: Suboptimal spleen response to ruxolitinib monotherapy (±placebo) of >0 but ≤35% (N=253; COMFORT-I, SIMPLIFY-1, TRANSFORM-1). Week-24 data used as a proxy to Week-18 suboptimal response assessment. SVR, spleen volume reduction, SVR25, spleen volume reduction ≥25%.

FIG. 14 illustrates suboptimal TSS responders based on a TSS30 response threshold, in accordance with an embodiment of the present disclosure. Utilizing the pooled dataset, it was determined among the JAKi-naive MF patients with a suboptimal response to ruxolitinib the mean TSS reduction was 28.5%. Studies Pooled: Suboptimal symptom response to ruxolitinib monotherapy (±placebo) of >0% but <50%. TSS, total symptom score, TSS30, ≥30% TSS reduction.

FIG. 15 illustrates the KRT-232-109 study, showing the spleen volume reduction after add-on navtemadlin to a stable dose of ruxolitinib, in accordance with an embodiment of the present disclosure. The KRT-232-109 study is a phase 2 study of add-on navtemadlin in patients with a suboptimal response to ruxolitinib. Note: Evaluable subjects with spleen volume assessments at baseline and Week 24 are shown. Baseline spleen volume magnetic resonance imaging/computed tomography scans were taken while subjects were on a stable dose of ruxolitinib for ≥8 weeks (e.g., no ruxolitinib wash-out). No dose increases of ruxolitinib above the stable baseline dose occurred during the 24-week assessment period. The median duration of ruxolitinib treatment prior to the addition of navtemadlin was 21.6 months (range: 7 to 129). *Six subjects discontinued treatment prior to Week 24. Abbreviations: ITT, intention-to-treat; Rux, ruxolitinib; yrs, years.

FIG. 16 illustrates a clinical trial (e.g., KRT-232-109 study), showing the total symptom score reduction after add-on navtemadlin to a stable dose of ruxolitinib, in accordance with an embodiment of the present disclosure. The KRT-232-109 study is a phase 2 study of add-on navtemadlin in patients with a suboptimal response to ruxolitinib. Note: Baseline TSS assessment was taken while subjects were on a stable dose of ruxolitinib for ≥8 weeks (e.g., no ruxolitinib wash-out). No dose increases of ruxolitinib above the stable baseline dose occurred during the 24-week assessment period. The median duration of ruxolitinib treatment prior to the addition of navtemadlin was 21.6 months (range: 7 to 129). *Six subjects discontinued treatment prior to Week 24. Abbreviations: MFSAF, Myelofibrosis Symptom Assessment Form; ITT, intention-to-treat; Rux, ruxolitinib; TSS, total symptom score; yrs, years.

FIG. 17 illustrates expected SVR for add-on navtemadlin to ruxolitinib, in accordance with an embodiment of the present disclosure. Spleen volume reductions modeled from KRT-232-109 and published phase 3 myelofibrosis studies. Note: Light gray bars depict the baseline distribution of SVR based on pooled, extractable data from published phase 3 JAKi-naïve MF studies for patients with suboptimal spleen response to ruxolitinib monotherapy (±placebo); COMFORT-1, SIMPLIFY-1, and TRANSFORM-1 (Verstovsek 2012; Mesa 2017; Pemmaraju 2023). Blue bars, regardless of shade, show the expected additional benefit of treating patients with add-on navtemadlin. Darkest blue bars depict SVR25 responders from a pre-randomization baseline that also cross the SVR35 threshold from a pre-ruxolitinib baseline (e.g., multi-component SVR endpoint). Abbreviations: CT, computed tomography; LOCF, last observation carried forward; MF, myelofibrosis; MRI, magnetic resonance imaging; Rux, ruxolitinib; SVR, splenic volume response; SVR25, spleen volume reduction ≥25%, SVR35, spleen volume reduction ≥35%.

FIG. 18 illustrates expected SVR for add-on placebo to ruxolitinib, in accordance with an embodiment of the present disclosure. Spleen volume reductions modeled from KRT-232-109 and published phase 3 myelofibrosis studies. Note: Light gray bars depict the baseline distribution of SVR based on pooled, extractable data from published phase 3 JAKi-naïve MF studies for patients with suboptimal spleen response to ruxolitinib monotherapy (±placebo); COMFORT-1, SIMPLIFY-1, and TRANSFORM-1 (Verstovsek 2012; Mesa 2017; Pemmaraju 2023). Dark gray bars show the expected additional benefit of treating patients with add-on placebo. No (0%) of patients are modeled to achieve the multi-component SVR endpoint of SVR25 from a pre-randomization baseline AND SVR35 from a pre-ruxolitinib baseline. Abbreviations: CT, computed tomography; LOCF, last observation carried forward; MF, myelofibrosis; MRI, magnetic resonance imaging; Rux, ruxolitinib; SVR, splenic volume response; SVR25, spleen volume reduction ≥25%, SVR35, spleen volume reduction ≥35%.

FIG. 19 illustrates expected TSS reduction add-on navtemadlin to ruxolitinib, in accordance with an embodiment of the present disclosure. Total symptom scores modeled from KRT-232-109 and published phase 3 myelofibrosis studies. Note: Light gray bars depict the baseline distribution of TSS reductions based on pooled extractable data from published phase 3 JAKi-naïve MF studies for patients with suboptimal symptom response to ruxolitinib monotherapy (±placebo); COMFORT-1, SIMPLIFY-1, and MANIFEST-2 (Verstovsek 2012; Mesa 2017; Rampal 2023). Green bars, regardless of shade, show the expected additional symptom improvement benefit of treating patients with add-on navtemadlin. Darkest green bars depict TSS30 responders from a pre-randomization baseline that also cross the TSS50 threshold from a pre-ruxolitinib baseline (multi-component TSS endpoint). Abbreviations: CT, computed tomography; LOCF, last observation carried forward; MF, myelofibrosis; MRI, magnetic resonance imaging; Rux, ruxolitinib; TSS, total symptoms score; TSS30, total symptom score reduction ≥30%, TSS50, total symptom score reduction ≥50%.

FIG. 20 illustrates expected TSS reductions for placebo add-on to ruxolitinib, in accordance with an embodiment of the present disclosure. Total symptom scores modeled from KRT-232-109 and published phase 3 myelofibrosis studies. Note: Light gray bars depict the baseline distribution of TSS reductions based on pooled extractable data from published phase 3 JAKi-naïve MF studies for patients with suboptimal symptom response to ruxolitinib monotherapy (±placebo); COMFORT-1, SIMPLIFY-1, and MANIFEST-2 (Verstovsek 2012; Mesa 2017; Rampal 2023). Darker gray bars show the expected additional benefit of treating patients with add-on placebo. Black bars depict TSS30 responders from a pre-randomization baseline that also cross the TSS50 threshold from a pre-ruxolitinib baseline (e.g., multi-component TSS endpoint). Abbreviations: CT, computed tomography; LOCF, last observation carried forward; MF, myelofibrosis; MRI, magnetic resonance imaging; Rux, ruxolitinib; TSS, total symptoms score; TSS30, total symptom score reduction ≥30%, TSS50, total symptom score reduction ≥50%.

### DETAILED DESCRIPTION OF THE INVENTION

While preferred embodiments of the invention are shown and described herein, such embodiments are provided by way of example only and are not intended to otherwise limit the scope of the invention. Various alternatives to the described embodiments of the invention may be employed in practicing the invention.

Unless defined otherwise, all technical and scientific terms used herein have the same meaning as is commonly understood by one of skill in the art to which this invention belongs.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

The terms “concurrent administration” or “co-administration” or “adjunct therapy” as used herein, encompass administration of two or more active pharmaceutical agents to a subject during the same period. This term implies that the treatments overlap in time, but it does not necessarily require that the pharmaceutical agents are taken at exactly the same moment or dosing interval. In some embodiments, the two pharmaceutical agents are administered to the subject at different dosing intervals and/or dosing regimen. In some embodiments, one pharmaceutical agent is administered before or after or at the same time as the other pharmaceutical agent. In one embodiment, the terms “adjunct therapy” and “add-on therapy” are interchangeable. The term “navtemadlin adjunct therapy” refers to adding navtemadlin to the existing therapy (e.g. ruxolitinib for treating myelofibrosis).

The term “combination” or “pharmaceutical combination” is defined herein to refer to either a fixed combination in one dosage unit form, a non-fixed combination or a kit of parts for the combined administration where the therapeutic agents may be administered together, independently at the same time or separately within time intervals, which preferably allows that the combination partners show a cooperative, e.g. synergistic effect. Thus, the single compounds of the pharmaceutical combination of the present disclosure could be administered simultaneously or sequentially.

Furthermore, the pharmaceutical combination of the present disclosure may be in the form of a fixed combination or in the form of a non-fixed combination.

The term “effective amount” or “therapeutically effective amount” refers to that amount of an active pharmaceutical ingredient or combination of active pharmaceutical ingredients as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, and other factors which can readily be determined by one of ordinary skill in the art. The term also applies to a dose that will induce a particular response in target cells, (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

In an embodiment, compounds described herein include of the isomers, stereoisomers, and enantiomers thereof.

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In selected embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve proton transfer between the cocrystal and the drug, and instead involves intermolecular interactions, such as hydrogen bonding, aromatic ring stacking, or dispersive forces, between the cocrystal former and the drug in the crystal structure.

The terms “QD,” “qd,” or “q.d.” means quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “ruxolitinib monotherapy” refers to the use of ruxolitinib as the only drug for treating patients with myelofibrosis.

A “therapeutic effect” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

Compounds of the invention also include crystalline and amorphous forms, including, for example, polymorphs, pseudopolymorphs, solvates, hydrates, unsolvated polymorphs (including anhydrates), conformational polymorphs, and amorphous forms of the compounds, as well as combinations thereof “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as combinations thereof, unless a particular crystalline or amorphous form is referred to.

Janus kinase (JAK) inhibitors, including the JAK 1/2 inhibitor ruxolitinib, have become the standard of care therapy for the treatment of patients with MF. However, many MF patients have a suboptimal response to ruxolitinib (e.g., SVR<35% and TSS reduction <50%). In this group, spleen volume remains enlarged, and symptom burden remains clinically relevant. There is an unmet need for improved therapies, with complementary mechanisms, that can be safely combined with a JAKi to treat patients with MF who have a suboptimal response to JAKi treatment. Effective novel therapies would enable more patients to achieve clinically meaningful reductions in splenomegaly and symptom burden and the potential for disease modification, with the goal of survival benefit.

MF is characterized by elevated circulating CD34+ progenitors that overexpress MDM2, due to upstream somatic gain-of-function mutations (e.g., JAK2 V617F) (Nakatake 2012; Orvain 2016). MDM2 is the key negative regulator of tumor suppressor protein 53 (p53). MDM2 overexpression by malignant progenitors is a mechanism by which these malignant CD34+ cells can evade normal p53 tumor suppressor detection and apoptosis.

Almost all patients with MF are TP53WT; just 2-4% of patients with chronic-phase MF carry a TP53 mutation (Harutyunyan 2011; Raza 2012), suggesting that overexpression of MDM2 is sufficient for MF progenitors to suppress normal p53 function, resulting in unchecked cancer cell proliferation. Therapeutic inhibition of MDM2 offers the potential to restore normal p53 function, leading to apoptosis of malignant MDM2-overexpressing MF progenitors, thereby complementing the immunomodulatory, anti-proliferative effects of backbone JAKi (e.g., ruxolitinib) therapy.

The apoptotic threshold of CD34+ MF progenitors can be influenced by factors including upstream pro-proliferative signaling (e.g., constitutive JAK-STAT activation), p21 induction of cell cycle arrest, and extrinsic pro-survival receptor signaling present within the supportive tumor microenvironment. Navtemadlin has potential for synergy with agents that lower the apoptotic threshold to enhance tumor cell death (Vousden 2007). Preclinical data have demonstrated that navtemadlin combined with ruxolitinib enhances apoptosis of MF patient-derived progenitors by lowering the apoptotic threshold (see, e.g., Example 3, below). The combination leverages complementary mechanisms converging on apoptotic cell death by inhibiting transient p21-mediated cell-cycle arrest and expression of pro-survival Bcl-2 family proteins (Clevenger 2023).

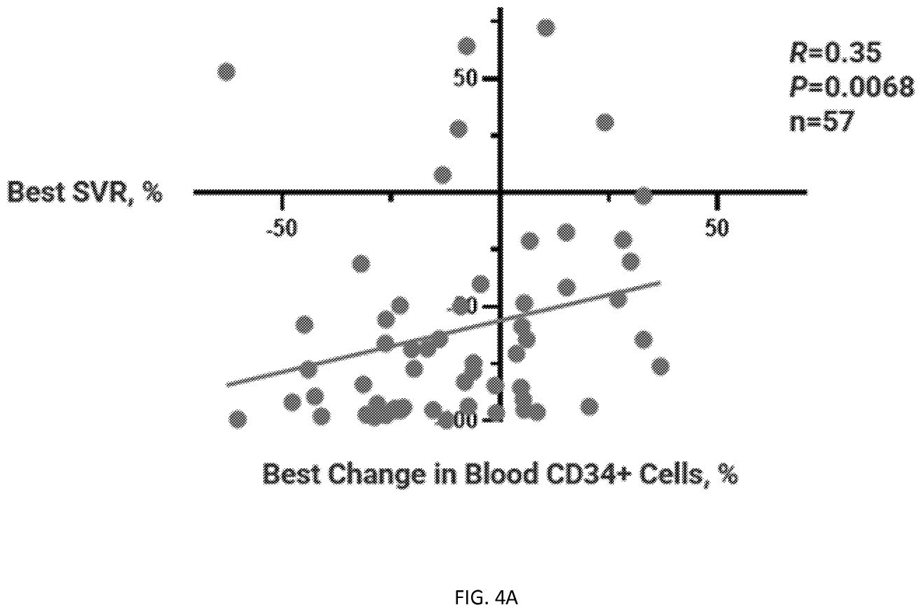

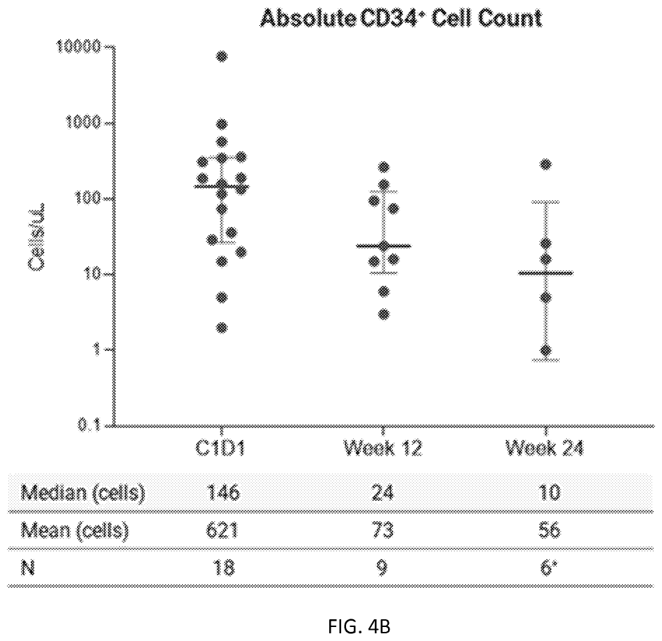

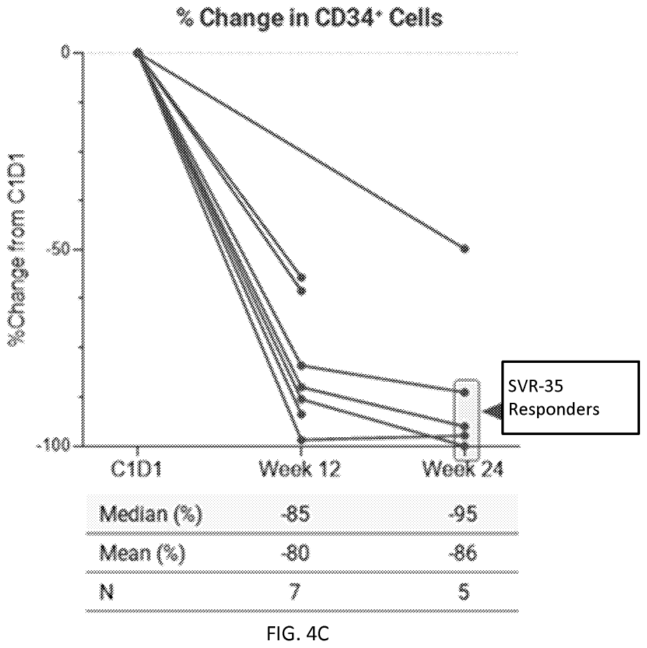

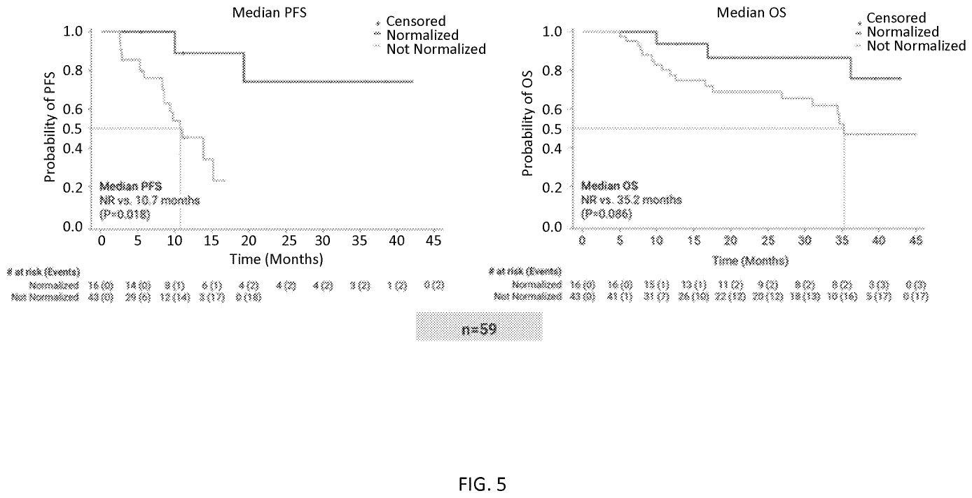

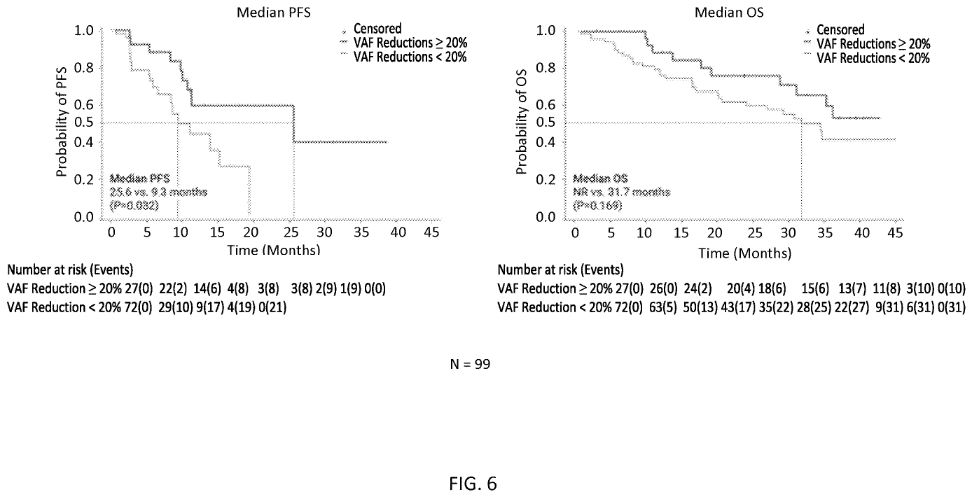

Ex vivo experiments demonstrated the synergistic cytotoxicity of navtemadlin and ruxolitinib (see, for instance, Examples 2-8, below) is clinically relevant and associated with improved clinical outcomes. Navtemadlin, both as a single agent in study KRT-232-101A, and as add-on therapy to ruxolitinib in study KRT-232-109, decreased circulating CD34+ cells (see, e.g., FIG. 4A and FIGS. 4B-C, respectively). In both studies, reduction in CD34+ cells correlated with SVR-patients with the largest decreases in peripheral CD34+ cell count had the largest reductions in spleen volume. As noted, reduction in spleen volume is associated with both increased OS, and improved symptom severity (Mesa 2013; Vannucchi 2015). These results establish the relevance of a cytotoxic mechanism for disease modification for navtemadlin as adjunct therapy to ruxolitinib that improves MF patient outcomes. Furthermore, this mechanism of action is distinct from and complementary to the mechanism through which ruxolitinib monotherapy acts (e.g., inhibition of the JAK-STAT signaling pathway).

Additionally, and without being limited to any particular theory, in some instances, there are beneficial effects of MDM2 inhibition toward the addictive feedback loop between stromal/microenvironment cells and the malignant clone. Because p53 activation reduces NF-κB-mediated signaling, it may synergize with ruxolitinib. In combination with JAK inhibition, p53 activation by navtemadlin may deprive the malignant clone of its proliferative “niche” within the bone marrow, and sites of extramedullary hematopoiesis. Synergistic anticancer effects suggest that add-on therapy of navtemadlin to ruxolitinib may be of particular benefit in patients with MF who have had a suboptimal response to ruxolitinib. As noted in Examples 2-8, interim results from the ongoing KRT-232-109 study demonstrated preliminary evidence of efficacy of navtemadlin as add-on therapy to ruxolitinib, providing clinical support for potential synergy in the therapeutic effects summarized above.

As described above, there is an unmet need for subjects with a suboptimal response to ruxolitinib. While ruxolitinib treatment results in SVR (e.g., >35%) and symptom control (e.g., TSS reduction of >50%) in a subset of patients, many patients have a suboptimal response (e.g., SVR<35% and TSS reduction <50%) (Harrison 2012; Verstovsek 2012; Mesa 2017). While this group of patients has, in some embodiments, minimal benefit from ongoing ruxolitinib use, spleen volume remains enlarged, and symptom burden remains clinically relevant. Thus, maximizing SVR is important to optimize patient outcomes because magnitude of SVR is correlated with, among other endpoints, improvements in quality of life, as assessed by TSS, and overall survival (OS). Without being limited to any one theory of operation, the smaller the spleen becomes, the greater the improvement in quality of life, and the longer an MF patient lives (Mesa 2013; Vannucchi 2015).

Traditional treatment options for human subjects suffering from myelofibrosis to improve outcomes for suboptimal ruxolitinib responders are limited. There are currently no drugs approved that may be added to ruxolitinib to optimize patient care. Unfortunately, as noted above, suboptimal response may also contribute to patient treatment discontinuation and patient outcomes after discontinuing ruxolitinib are dismal. In patients who discontinue ruxolitinib, median OS was just 13.2 months (95% confidence interval [CI], 8.0-22.7 months) (Newberry 2017). Together, these data clearly show the unmet medical need for improved treatment options in MF patients with suboptimal response to ruxolitinib.

### Concurrent Administration

In one aspect, the present invention relates to a method for treating myelofibrosis in a human subject who has been previously treated for myelofibrosis with ruxolitinib monotherapy and had a suboptimal response to said previous treatment with ruxolitinib monotherapy comprising concurrent administration to the human of a therapeutically effective amount of navtemadlin or a pharmaceutically acceptable salt thereof and ruxolitinib.

The term “suboptimal response” is defined as, after the ruxolitinib monotherapy for treating MF, SVR<35% by MRI/CT scan (central review) and TSS reduction of <50%, assessed from the start of the ruxolitinib monotherapy to the end of ruxolitinib monotherapy (“monotherapy period”). In some embodiments, suboptimal response refers to SVR of >−25% but <35% by MRI/CT scan (central review) and TSS reduction of >−35% but <50%. In some embodiments, suboptimal response refers to SVR of >−20% but <35% by MRI/CT scan (central review) and TSS reduction of >−30% but <50%. In some embodiments, suboptimal response refers to SVR of >−15% but <35% by MRI/CT scan (central review) and TSS reduction of >−25% but <50%. In some embodiments, suboptimal response refers to SVR of >−10% but <35% by MRI/CT scan (central review) and TSS reduction of >−20% but <50%. In some embodiments, suboptimal response refers to SVR of >−5% but <35% by MRI/CT scan (central review) and TSS reduction of >−15% but <50%. In some embodiments, suboptimal response refers to SVR of >0% but <35% by MRI/CT scan (central review) and TSS reduction of >−5% but <50%.

In some embodiments, suboptimal response refers to SVR of >0% but <35% by MRI/CT scan (central review) and TSS reduction of >0% but <50%. In some embodiments, suboptimal response refers to SVR of >0% but <20% by MRI/CT scan (central review) and TSS reduction of >0% but <30%. In some embodiments, suboptimal response refers to SVR of ≥20% but <35% by MRI/CT scan (central review) and TSS reduction of ≥30% but <50%. In some embodiments, suboptimal response refers to SVR of >0% but <15% by MRI/CT scan (central review) and TSS reduction of >0% but <25%. In some embodiments, suboptimal response refers to SVR of >0% but <25% by MRI/CT scan (central review) and TSS reduction of >0% but <35%. In some embodiments, suboptimal response refers to SVR of >0% but <10% by MRI/CT scan (central review) and TSS reduction of >0% but <25%. When the SVR is negative (for example, −10%), it indicates that the spleen volume has increased by 10%. When the TSS is negative (for example, −10%), it indicates that the TSS has increased by 10%.

In some embodiments, the spleen volume reduction (SVR) by MRI/CT scan (central review) in the human after the previous treatment with ruxolitinib monotherapy is <35% compared to the spleen volume prior to the previous treatment with ruxolitinib monotherapy; and the total symptom score (TSS) of the human after the previous treatment with ruxolitinib monotherapy is less than 50% compared to a TSS prior to the previous treatment with ruxolitinib monotherapy.

In some embodiments, the spleen volume reduction (SVR) by MRI/CT scan (central review) in the human after the previous treatment with ruxolitinib monotherapy is >zero but <35% compared to the spleen volume prior to the previous treatment with ruxolitinib monotherapy; and the total symptom score (TSS) of the human after the previous treatment with ruxolitinib monotherapy is >zero but less than 50% compared to a TSS prior to the previous treatment with ruxolitinib monotherapy.

In some embodiments, the spleen volume reduction (SVR) by MRI/CT scan (central review) in the human after the previous treatment with ruxolitinib monotherapy is >zero but <20% compared to the spleen volume prior to the previous treatment with ruxolitinib monotherapy; and the total symptom score (TSS) of the human after the previous treatment with ruxolitinib monotherapy is >zero but less than 30% compared to a TSS prior to the previous treatment with ruxolitinib monotherapy.

In some embodiments, the spleen volume reduction (SVR) by MRI/CT scan (central review) in the human after the previous treatment with ruxolitinib monotherapy is ≥20% but <35% compared to the spleen volume prior to the previous treatment with ruxolitinib monotherapy; and the total symptom score (TSS) of the human after the previous treatment with ruxolitinib monotherapy is ≥30% but <50% compared to a TSS prior to the previous treatment with ruxolitinib monotherapy.

In some embodiments, the method comprises two periods: the monotherapy period, as stated above, and the co-administration period when navtemadlin or a pharmaceutically acceptable salt thereof and ruxolitinib are concurrently administered to the human.

At the start of ruxolitinib monotherapy, or prior to initiating ruxolitinib monotherapy, the human subject's spleen volume and TSS are measured. This is called the first measurement. After the ruxolitinib monotherapy before the co-administration period, the human subject's spleen volume and TSS are measured again. This is called the second measurement. SVR and TSS reduction are calculated based on the first measurement and the second measurement. In some embodiments, during the ruxolitinib monotherapy period, SVR is about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1%. In some embodiments, during the monotherapy period, SVR is within a range of about 1-35%, about 5-30%, about 10-20%, about 10-40%, about 0-20%, or about 20-35%, inclusive. In some embodiments, during the ruxolitinib monotherapy period, SVR is within a range of 0-35%, 10-35%, 20-35%, 10-20% or 10-30%, inclusive. In some embodiments, during the ruxolitinib monotherapy period, TSS reduction is about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1%. In some embodiments, during the ruxolitinib monotherapy period, TSS reduction is about 95%, about 90%, about 85%, about 80%, about 75%, about 70%, about 65%, about 60%, about 55%, or about 50%. In some embodiments, during the ruxolitinib monotherapy period, TSS reduction is within a range of 0-50%, 0-40%, 0-30%, 5-45%, 5-40%, 5-30%, 10-40%, 15-40%, 30-50%, inclusive. In some embodiments, during the ruxolitinib monotherapy period, TSS reduction is within a range of 0-50%, 10-50%, 20-50%, 30-50^{%}, 10-40%, 10-30%, 20-30%, or 15-45%. In some embodiments, the human subject has a suboptimal response to the ruxolitinib monotherapy for treating MF, wherein SVR is <35% and TSS reduction is <50%.

In some embodiments, the human has a refractory MF or a relapsed MF. In some embodiments, the human has not been previously treated with a JAK inhibitor prior to ruxolitinib monotherapy. In some embodiments, the human is JAK inhibitor-naïve prior to ruxolitinib monotherapy.

In some embodiments, the human subject has a suboptimal response to the ruxolitinib monotherapy for treating MF, wherein SVR is <30% and TSS reduction is <45%. In some embodiments, the human subject has a suboptimal response to the ruxolitinib monotherapy for treating MF, wherein SVR is <25% and TSS reduction is <40%. In some embodiments, the human subject has a suboptimal response to the ruxolitinib monotherapy for treating MF, wherein SVR is <20% and TSS reduction is <35%. In some embodiments, the human subject has a suboptimal response to the ruxolitinib monotherapy for treating MF, wherein SVR is <15% and TSS reduction is <30%. In some embodiments, the human subject has a suboptimal response to the ruxolitinib monotherapy for treating MF, wherein SVR is <10% and TSS reduction is <25%. In some embodiments, the human subject has a suboptimal response to the ruxolitinib monotherapy for treating MF, wherein SVR is <5% and TSS reduction is <20%. In some embodiments, the human subject has a suboptimal response to the ruxolitinib monotherapy for treating MF, wherein SVR is <20% and TSS reduction is <30%.

The TSS is determined using a Myelofibrosis Symptom Assessment Form (MFSAF) and where the comparison determines a reduction in total symptom score between the baseline measurement (the first measurement) and after the ruxolitinib monotherapy (the second measurement). See, e.g., Mesa et al. The Myelofibrosis Symptom Assessment Form (MFSAF): An Evidence-based Brief Inventory to Measure Quality of Life and Symptomatic Response to Treatment in Myelofibrosis. Leuk Res. 2009 September; 33(9): 1199-1203. doi:10.1016/j.leukres.2009.01.035, which is hereby incorporated herein by reference in its entirety.

In some embodiments, the MFSAF shown below is used for determining TSS. Items:

In some embodiments, the duration of the ruxolitinib monotherapy is at least 8 weeks, 12 weeks, 14 weeks, 16 weeks, 18 weeks, 20 weeks, 24 weeks, 28 weeks, 32 weeks, 36 weeks, 40 weeks, 44 weeks, 48 weeks, 52 weeks, or 56 weeks. In some embodiments, the duration of the ruxolitinib monotherapy is at least 12 weeks. In some embodiments, the duration of the ruxolitinib monotherapy is at least 18 weeks.

In some embodiments, the duration of the ruxolitinib monotherapy can be at least 1 month, 2 months, 3 months, 4 months, 5 months, 6 months, 7 months, 8 months, 9 months, 10 months, 11 months, one year, two, three, or four years.

In some embodiments, ruxolitinib is administered at a dose of 5 mg, 10 mg, 15 mg, 20 mg, or 25 mg twice daily during the ruxolitinib monotherapy. In some embodiments, the dose for administration may be adjusted based on the human's response during the ruxolitinib monotherapy, for example, increasing from 5 mg twice daily to 10 mg twice daily.

In some embodiments, ruxolitinib is administered at a dose of 5 mg twice daily during the ruxolitinib monotherapy.

In some embodiments, ruxolitinib is administered at a dose of 10 mg twice daily during the ruxolitinib monotherapy.

In some embodiments, ruxolitinib is administered at a dose of 15 mg twice daily during the ruxolitinib monotherapy.

In some embodiments, ruxolitinib is administered at a dose of 20 mg twice daily during the ruxolitinib monotherapy.

In some embodiments, ruxolitinib is administered at a dose of 25 mg twice daily during the ruxolitinib monotherapy.

In some embodiments, ruxolitinib is administered at a stable dose during the ruxolitinib monotherapy. The term “stable dose” refers administration of the same dose of ruxolitinib for a period of time. In some embodiments, such period of time can be at least 8 weeks, 12 weeks, 16 weeks, 20 weeks, 24 weeks, 28 weeks, 32 weeks, 36 weeks, 40 weeks, 44 weeks, 48 weeks, 52 weeks, 56 weeks, one year, two years, or three years. In some embodiments, the stable dose is 5 mg, 10 mg, 15 mg, 20 mg, or 25 mg twice daily during the ruxolitinib monotherapy. In some embodiments, the stable dose is 5 mg, 10 mg, 15 mg, 20 mg, or 25 mg twice daily during the ruxolitinib monotherapy. In some embodiments, the stable dose is 5 mg twice daily during the ruxolitinib monotherapy. In some embodiments, the stable dose is 10 mg twice daily during the ruxolitinib monotherapy. In some embodiments, the stable dose is 15 mg twice daily during the ruxolitinib monotherapy. In some embodiments, the stable dose is 20 mg twice daily during the ruxolitinib monotherapy. In some embodiments, the stable dose is 25 mg twice daily during the ruxolitinib monotherapy.

In some embodiments, navtemadlin or a pharmaceutically acceptable salt thereof is administered as an add-on therapy or adjunct therapy. The term “adjunct therapy” or “add-on therapy” refers to a treatment that is prescribed in addition to the initial therapy to enhance therapeutic effects, manage symptoms, or address conditions that are not adequately controlled by the initial therapy. In some embodiments, navtemadlin or a pharmaceutically acceptable salt thereof is administered as an adjunct therapy, wherein ruxolitinib is continued to be administered. In some embodiments, navtemadlin or a pharmaceutically acceptable salt thereof and ruxolitinib are concurrently administered to the human. This duration is referred to as the co-administration period or concurrent administration period. In some embodiments, the co-administration period is at least 4 weeks, 8 weeks, 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks, 76 weeks, 80 weeks, 84 weeks, 88 weeks, 92 weeks, 96 weeks, 100 weeks, or 104 weeks. In some embodiments, the co-administration period is about one month, two months, three months, four months, five months, six months, seven months, eight months, nine months, ten months or eleven months. In some embodiments, the co-administration period is about one, two, three, four, or five years.

In some embodiments, during the co-administration period, navtemadlin or a pharmaceutically acceptable salt thereof is administered on a 28-day treatment cycle. Navtemadlin or a pharmaceutically acceptable salt thereof is administered on days 1-7 of the 28-day treatment cycle, while navtemadlin or a pharmaceutically acceptable salt thereof is not administered on days 8-28 of the 28-day treatment cycle.

In some embodiments, during the co-administration period, navtemadlin or a pharmaceutically acceptable salt thereof is administered on a 21-day treatment cycle. Navtemadlin or a pharmaceutically acceptable salt thereof is administered on days 1-7 of the 21-day treatment cycle, while navtemadlin or a pharmaceutically acceptable salt thereof is not administered on days 8-21 of the 21-day treatment cycle.

In some embodiments, during the co-administration period, navtemadlin or a pharmaceutically acceptable salt thereof is administered for at least one, two, three, four, five, six, seven, eight, nine, ten, or more treatment cycles. In some embodiments, the co-administration period aligns with the treatment cycles during which navtemadlin or a pharmaceutically acceptable salt thereof is administered.

In some embodiments, the spleen volume reduction (SVR) in the human after the one or more treatment cycles of navtemadlin adjunct therapy is at least 35%, 40%, 45%, 50%, 55%, 60%, 65%, 70%, 75%, 80%, 85%, 90%, or 95% compared to the spleen volume prior to the ruxolitinib monotherapy.

In some embodiments, the TSS in the human after the one or more treatment cycles of navtemadlin adjunct therapy is reduced by at least 30%, 40%, 45%, 50%, 55%, 60%, 65%, 70%, 75%, 80%, 85%, 90%, or 95% compared to a TSS prior to the one or more treatment cycles.

In some embodiments, the TSS of the human after the one or more treatment cycles of navtemadlin adjunct therapy is reduced by at least 50%, 55%, 60%, 65%, 70%, 75%, 80%, 85%, 90%, or 95% compared to a TSS prior to the ruxolitinib monotherapy.

In some embodiments, the SVR in the human after the one or more treatment cycles of navtemadlin adjunct therapy is at least 25% compared to the spleen volume prior to the one or more treatment cycles and at least 35% compared to the spleen volume prior to the ruxolitinib monotherapy.

In some embodiments, the TSS of the human after the one or more treatment cycles of navtemadlin adjunct therapy is reduced by at least 30% compared to a TSS prior to the one or more treatment cycles and at least 50% compared to the TSS prior to the ruxolitinib monotherapy.

In some embodiments, the SVR in the human after the one or more treatment cycles of navtemadlin adjunct therapy is at least 25% compared to the spleen volume prior to the one or more treatment cycles and at least 35% compared to the spleen volume prior to the ruxolitinib monotherapy; and the TSS of the human after the one or more treatment cycles is reduced by at least 30% compared to a TSS prior to the one or more treatment cycles and at least 50% compared to the TSS prior to the ruxolitinib monotherapy.

In some embodiments, the spleen volume reduction (SVR) in the human after the co-administration of navtemadlin and ruxolitinib is at least 35%, 40%, 45%, 50%, 55%, 60%, 65%, 70%, 75%, 80%, 85%, 90%, or 95% compared to the spleen volume prior to the ruxolitinib monotherapy.

In some embodiments, the TSS in the human after the co-administration of navtemadlin and ruxolitinib is reduced by at least 30%, 40%, 45%, 50%, 55%, 60%, 65%, 70%, 75%, 80%, 85%, 90%, or 95% compared to a TSS prior to the co-administration.

In some embodiments, the TSS of the human after the co-administration of navtemadlin and ruxolitinib is reduced by at least 50%, 55%, 60%, 65%, 70%, 75%, 80%, 85%, 90%, or 95% compared to a TSS prior to the ruxolitinib monotherapy.

In some embodiments, the SVR in the human after the co-administration of navtemadlin and ruxolitinib is at least 25% compared to the spleen volume prior to the co-administration and at least 35% compared to the spleen volume prior to the ruxolitinib monotherapy.

In some embodiments, the TSS of the human after the co-administration of navtemadlin and ruxolitinib is reduced by at least 30% compared to a TSS prior to the co-administration and at least 50% compared to the TSS prior to the ruxolitinib monotherapy.

In some embodiments, the SVR in the human after the co-administration of navtemadlin and ruxolitinib is at least 25% compared to the spleen volume prior to the co-administration and at least 35% compared to the spleen volume prior to the ruxolitinib monotherapy; and the TSS of the human after the co-administration is reduced by at least 30% compared to a TSS prior to the co-administration and at least 50% compared to the TSS prior to the ruxolitinib monotherapy.

In a preferred embodiment, the SVR in the human after the co-administration of navtemadlin and ruxolitinib is at least 25% compared to the spleen volume prior to the co-administration and at least 35% compared to the spleen volume prior to the ruxolitinib monotherapy; and the TSS of the human after the co-administration is reduced by at least 30% compared to a TSS prior to the co-administration and at least 50% compared to the TSS prior to the ruxolitinib monotherapy.

In some embodiments, the spleen volume reduction (SVR) in the human after the previous treatment with ruxolitinib monotherapy is <35% compared to the spleen volume prior to the ruxolitinib monotherapy; wherein the total symptom score (TSS) of the human after the ruxolitinib monotherapy is less than 50% compared to a TSS prior to the ruxolitinib monotherapy.

In some embodiments, navtemadlin or a pharmaceutically acceptable salt thereof is administered at a dose of 240 mg daily. In some embodiments, navtemadlin or a pharmaceutically acceptable salt thereof is administered at a dose of 120 mg daily. In some embodiments, navtemadlin or a pharmaceutically acceptable salt thereof is administered at a dose of 240 mg twice daily. In some embodiments, navtemadlin or a pharmaceutically acceptable salt thereof is administered at a dose of 120 mg twice daily. In some embodiments, the navtemadlin or a pharmaceutically acceptable salt thereof is administered at a total dose of between 150 mg and 300 mg per day.

In some embodiments, ruxolitinib is administered at a dose of 5 mg, 10 mg, 15 mg, 20 mg, or 25 mg twice daily during the co-administration period.

In some embodiments, ruxolitinib is administered at a dose of 5 mg twice daily during the co-administration period.

In some embodiments, ruxolitinib is administered at a dose of 10 mg twice daily during the co-administration period.

In some embodiments, ruxolitinib is administered at a dose of 15 mg twice daily during the co-administration period.

In some embodiments, ruxolitinib is administered at a dose of 20 mg twice daily during the co-administration period.

In some embodiments, ruxolitinib is administered at a dose of 25 mg twice daily during the co-administration period.

In some embodiments, the human has JAK2 mutation. In some embodiments, the human has a JAK2V617F mutation. In some embodiments, the human does not have a JAK2V617F mutation.

In some embodiments, the human does not have TP53 mutation. In some embodiments, the human has one or more benign TP53 mutations.

In some embodiments, the human has CALR mutation. In some embodiments, the human has CALR mutation, JAK2 mutation, and/or BCR-ABL1 mutation.

In some embodiments, the human has MPL mutation. In some embodiments, the human has CALR mutation, JAK2 mutation, MPL mutation, and/or BCR-ABL1 mutation.

In another aspect, the present invention relates to a method for treating myelofibrosis in a human subject who has been previously treated for myelofibrosis with ruxolitinib monotherapy and had a suboptimal response to said ruxolitinib monotherapy comprising the steps of: i) conducting a first measurement measuring a first spleen volume and a first TSS of a human with myelofibrosis; ii) administering to the human in need thereof ruxolitinib for a period of time (ruxolitinib monotherapy); iii) conducting a second measurement measuring a second spleen volume and a second TSS of the human after the period of treatment with ruxolitinib monotherapy; iv) administering navtemadlin or a pharmaceutically acceptable salt thereof concurrently with ruxolitinib to the human if the second spleen volume is reduced by <35% compared to the first spleen volume and the second TSS is reduced by less than 50% compared to the first TSS. In some embodiments, the period of time is at least about 18 weeks. In some embodiments, the period of time is at least about 19 weeks. In some embodiments, the period of time is at least about 20 weeks. In some embodiments, the period of time is at least about 21 weeks. In some embodiments, the period of time is at least about 22 weeks. In some embodiments, the period of time is at least about 23 weeks. In some embodiments, the period of time is at least about 24 weeks. In some embodiments, the period of time is at least about 25 weeks. In some embodiments, the duration of the ruxolitinib monotherapy is at least 8 weeks, 12 weeks, 14 weeks, 16 weeks, 18 weeks, 20 weeks, 24 weeks, 28 weeks, 32 weeks, 36 weeks, 40 weeks, 44 weeks, 48 weeks, 52 weeks, or 56 weeks.

In some embodiments, the duration of the ruxolitinib monotherapy is at least 1 month, 2 months, 3 months, 4 months, 5 months, 6 months, 7 months, 8 months, 9 months, 10 months, 11 months, one year, two, three, or four years.

In some embodiments, during the co-administration period (concurrent administration period), navtemadlin or a pharmaceutically acceptable salt thereof is administered on a 28-day treatment cycle. Navtemadlin or a pharmaceutically acceptable salt thereof is administered on days 1-7 of the 28-day treatment cycle, while navtemadlin or a pharmaceutically acceptable salt thereof is not administered on days 8-28 of the 28-day treatment cycle. In some embodiments, during the co-administration period (concurrent administration period), navtemadlin or a pharmaceutically acceptable salt thereof is administered on a 21-day treatment cycle. Navtemadlin or a pharmaceutically acceptable salt thereof is administered on days 1-7 of the 21-day treatment cycle, while navtemadlin or a pharmaceutically acceptable salt thereof is not administered on days 8-21 of the 21-day treatment cycle. In some embodiments, during the co-administration period, navtemadlin or a pharmaceutically acceptable salt thereof is administered for at least one, two, three, four, five, six, seven, eight, nine, ten, or more treatment cycles.

In some embodiments, navtemadlin or a pharmaceutically acceptable salt thereof is administered for one or more treatment cycles concurrently with ruxolitinib. In some embodiments, the co-administration period aligns with the treatment cycles during which navtemadlin or a pharmaceutically acceptable salt thereof is administered.

In some embodiments, the method further comprises a step of conducting a third measurement measuring a third spleen volume and a third total symptom score of the human after the one or more treatment cycles.

In some embodiments, the third spleen volume after the one or more treatment cycles is reduced by at least 35% compared to the first spleen volume and by at least 25% compared to the second spleen volume.

In some embodiments, the third TSS after the one or more treatment cycles is reduced by at least 50% compared to the first TSS and by at least 30% compared to the second TSS.

In some embodiments, the third spleen volume after the one or more treatment cycles is reduced by at least 35% compared to the first spleen volume and by at least 25% compared to the second spleen volume; wherein the third TSS is reduced by at least 50% compared to the first TSS and by at least 30% compared to the second TSS.

In a preferred embodiment, navtemadlin or a pharmaceutically acceptable salt thereof is administered for one or more treatment cycles concurrently with ruxolitinib, wherein the third spleen volume after the one or more treatment cycles is reduced by at least 35% compared to the first spleen volume and by at least 25% compared to the second spleen volume; wherein the third TSS is reduced by at least 50% compared to the first TSS and by at least 30% compared to the second TSS.

In yet another aspect, the present disclosure relates to a method of treating MF in a human in need thereof. Such method comprises administering to the human a pharmaceutically effective amount of a MDM2 inhibitor as an add-on therapy to the existing JAKi monotherapy, where the human has a suboptimal response to the JAKi monotherapy. In some embodiments, the MDM2 inhibitor is described in the section of “MDM2 inhibitors”. In some embodiments, JAKi is described in the section of “JAK inhibitors”. In some embodiments, the MDM2 inhibitor is navtemadlin, or a pharmaceutically acceptable salt thereof. In some embodiments, the JAKi is ruxolitinib.

In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, R06839921, APG-115, MI-1601, and pharmaceutically acceptable salts thereof.

In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, R06839921, APG-115, MI-1601, and pharmaceutically acceptable salts thereof.

In an embodiment, the JAK inhibitor is selected from the group consisting of AC-410, AT9283, AZ960, AZD-1480, Baricitinib, BMS-911543, CEP-33779, Cerdulatinib, CHZ868, CYT387, Decernotinib, ENMD-2076, Filgotinib, Ganetespib, INCB039110, INCB-047986, Itacitinib, JAK3-IN-1, JANEX-1, LFM-A13, LY2784544, NS-018, NSC42834, NVP-BSK805, Oclacitinib, Pacritinib, Peficitinib, Pyridone 6, R348, RGB-286638, Ruxolitinib, Ruxolitinib-S, SAR-20347, SB1317, Solcitinib, TG101209, TG101348, Tofacitinib(3R,4S), Tofacitinib(3S,4R), Tofacitinib(3S,4S), Tofacitinib, TYK2-IN-2, Upadacitinib, WHI-P154, WHI-P97, WP1066, XL019, ZM39923, and pharmaceutically acceptable salts thereof.

In an embodiment, the JAK inhibitor is selected from the group consisting of Baricitinib phosphate, CYT387 Mesylate, CYT387 sulfate salt, NS-018 hydrochloride, NS-018 maleate, NVP-BSK805 dihydrochloride, Oclacitinib maleate, Ruxolitinib phosphate, Ruxolitinib sulfate, Tofacitinib citrate, and ZM39923 hydrochloride.

### Navtemadlin

Navtemadlin is a potent and selective, small molecule mouse double minute 2 (MDM2) inhibitor that binds human MDM2 to neutralize its interaction with p53. This restores p53-mediated activity and induces apoptosis in TP53 wild-type (WT) malignancies (Sun 2014). The primary determinant of sensitivity to MDM2 inhibition is TP53 mutation status. Specifically, if tumors have TP53 mutations that impair the transcriptional activity of p53, then MDM2 inhibition may not modulate the tumor suppressor functions of p53. Therefore, the safety and efficacy of navtemadlin treatment should only be evaluated in patients with TP53WT tumors. To this point, strategies to maximize efficacy of MDM2i may be most apparent in tumor cells that have evaded normal p53 tumor suppressor function through upregulating MDM2 levels (e.g., MDM2 gene amplification, MDM2 overexpression, or MDM2 nuclear protein upregulation).

Non-clinical pharmacology studies have shown that navtemadlin drives a dose-dependent reduction in tumor growth in xenograft models representing various genetic backgrounds, and tumor types harboring TP53WT. Activation of the p53 pathway was observed with navtemadlin treatment as measured by increases in cyclin-dependent kinase inhibitor protein (p21) mRNA, a direct transcriptional target of p53, and induction of pro-apoptosis proteins leading to tumor cell death. Preclinical studies conducted with navtemadlin have yielded promising results in in vitro, ex vivo, and in vivo experiments, representing solid tumors and hematologic malignancies, including MF and acute myeloid leukemia secondary to an MPN.

Repeat-dose toxicity studies of up to 13 weeks duration in rats and monkeys, along with in vitro and in vivo safety pharmacology studies, have been completed for navtemadlin. In a standard panel of assays, navtemadlin was not mutagenic or genotoxic, and had negligible potential for phototoxicity. Nonclinical embryofetal toxicity and fertility studies have not been conducted for navtemadlin due to the well documented requirement for p53/MDM2 control during embryonic and fetal development. Based on its mechanism of action, navtemadlin alters cell proliferation and is expected to result in adverse effects on embryofetal viability and development.

### MDM2 Inhibitors

The compound of Formula (I) has the structure and name shown below. 2-((3R,5R,6S)-5-(3-chlorophenyl)-6-(4-chlorophenyl)-1-((S)-1-(isopropylsulfonyl)-3-methylbutan-2-yl)-3-methyl-2-oxopiperidin-3-yl) acetic acid:

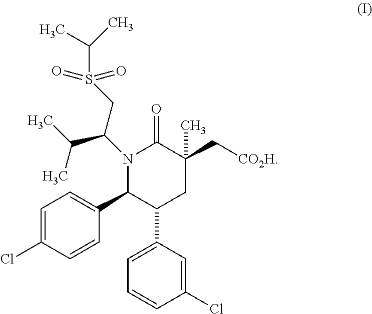

The synthesis of the compound of Formula (I) is set forth in International Applications: WO2011/153509 and WO2014/200937; U.S. Pat. Nos. 8,569,341; 9,593,129; 9,296,736; 9,623,018; 9,757,367; 9,801,867; 9,376,386; and 9,855,259, the disclosure of which are incorporated by reference herein in its entirety.

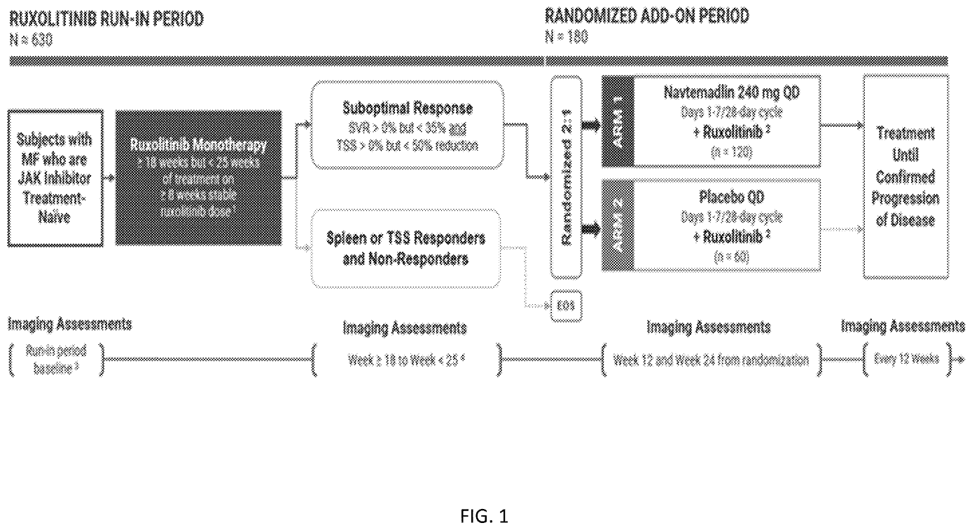

In an embodiment, the compound of Formula (I) or Formula (II) is in an amorphous form. In an embodiment, the MDM2 inhibitor is the compound of Formula (I) or Formula (II) in a crystalline form. In an embodiment, the MDM2 inhibitor is the compound of Formula (I) in a crystalline anhydrous form. In an embodiment, the MDM2 inhibitor is the compound of Formula (I) in a crystalline anhydrous form characterized by a powder X-ray diffraction pattern comprising peaks at diffraction angle 2 theta degrees at approximately 11.6, 12.4, 18.6, 19.0, 21.6 and 23.6. In an embodiment, the MDM2 inhibitor is the compound of Formula (I) in a crystalline anhydrous form having the X-ray diffraction pattern substantially shown in FIG. 1. The method of making such crystalline form was disclosed in the International Application WO2014200937, the disclosure of which is incorporated herein by reference in its entirety.

In an embodiment, the MDM2 inhibitor is a compound of Formula (II) having the structure and name shown below.

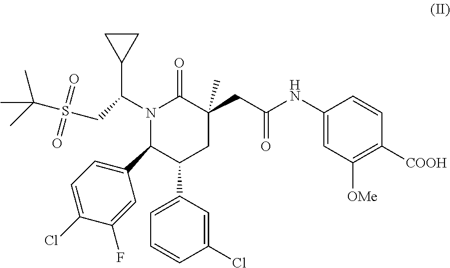 [SMILES: COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

### 4-(2-((3R,5R,6S)-1-((S)-2-(tert-butylsulfonyl)-1-cyclopropylethyl)-6-(4-chloro-3-fluorophenyl)-5-(3-chlorophenyl)-3-methyl-2-oxopiperidin-3-yl)acetamido)-2-methoxybenzoic acid

The synthesis of the compound of Formula (II) is set forth in U.S. Pat. No. 8,952,036, the disclosure of which is incorporated by reference herein in its entirety.

### RG7388 (Idasanutlin)

In an embodiment, the MDM2 inhibitor is RG7388. RG7388 has the chemical structure and name shown as:

### 4-[[(2R,3S,4R,5S)-3-(3-chloro-2-fluorophenyl)-4-(4-chloro-2-fluorophenyl)-4-cyano-5-(2,2-dimethylpropyl)pyrrolidine-2-carbonyl]amino]-3-methoxybenzoic acid

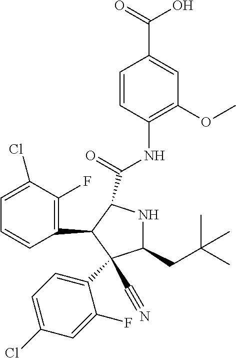 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

### Triptolide (PG490)

In an embodiment, the MDM2 inhibitor is triptolide. Triptolide has the chemical structure and name shown as:

### (5bS,6aS,7aS,8R,8aR,9aS,9bS,10aS,10bS)-8-hydroxy-8a-isopropyl-10b-methyl-2,5,5b,6,6a,8,8a,9a,9b,10b-decahydrotris(oxireno) [2′,3′:4b,5; 2″,3″:6,7; 2″′,3″′:8a,9]phenanthro[1,2-c]furan-3(1H)-one

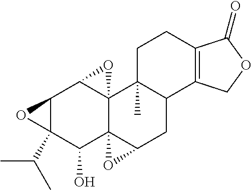

### Nutlin-3a

In an embodiment, the MDM2 inhibitor is Nutlin-3a. Nutlin-3a has the chemical structure and name shown as:

### 4-[(4S,5R)-4,5-bis(4-chlorophenyl)-2-(4-methoxy-2-propan-2-yloxyphenyl)-4,5-dihydroimidazole-1-carbonyl]piperazin-2-one

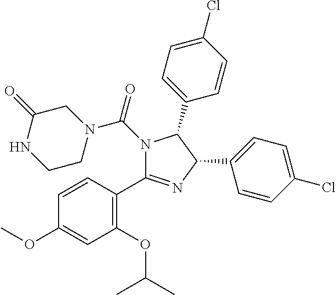 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

In an embodiment, the MDM2 inhibitor is HDM201. HDM201 has the chemical structure and name shown as:

### (4S)-5-(5-chloro-1-methyl-2-oxopyridin-3-yl)-4-(4-chlorophenyl)-2-(2,4-dimethoxypyrimidin-5-yl)-3-propan-2-yl-4H-pyrrolo[3,4-d]imidazol-6-one

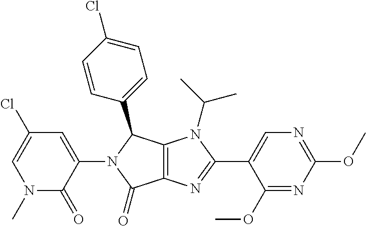 [SMILES: COc1ncc(-c2nc3c(n2C(C)C)[C@H](c2ccc(Cl)cc2)N(c2cc(Cl)cn(C)c2=O)C3=O)c(OC)n1]

### RG7112

In an embodiment, the MDM2 inhibitor is RG7112. RG7112 has the chemical structure and name shown as:

### [(4S,5R)-2-(4-tert-butyl-2-ethoxyphenyl)-4,5-bis(4-chlorophenyl)-4,5-dimethylimidazol-1-yl]-[4-(3-methylsulfonylpropyl)piperazin-1-yl]methanone

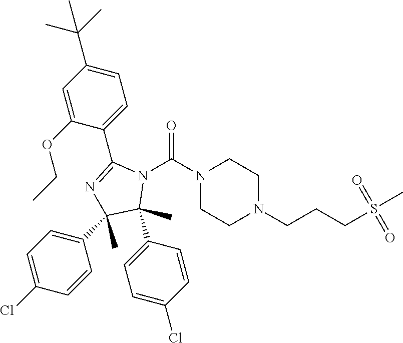 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

### CGM097A

In an embodiment, the MDM2 inhibitor is CGM097A. CGM097A has the chemical structure and name shown as:

### (1S)-1-(4-chlorophenyl)-6-methoxy-2-[4-[methyl-[[4-(4-methyl-3-oxopiperazin-1-yl)cyclohexyl]methyl]amino]phenyl]-7-propan-2-yloxy-1,4-dihydroisoquinolin-3-one

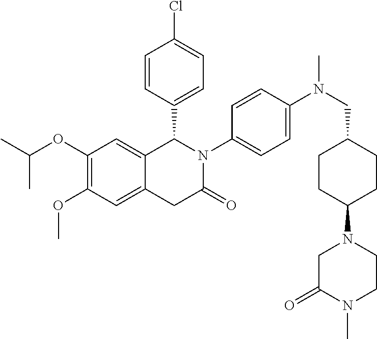 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2]

### Nutlin-3

In an embodiment, the MDM2 inhibitor is nutlin-3. Nutlin-3 has the chemical structure and name shown as:

### 4-[4,5-bis(4-chlorophenyl)-2-(4-methoxy-2-propan-2-yloxyphenyl)-4,5-dihydroimidazole-1-carbonyl]piperazin-2-one

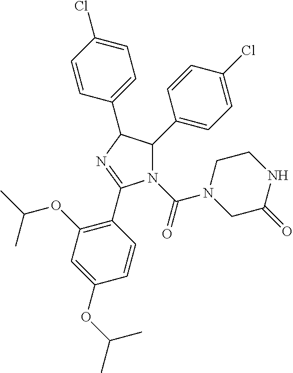 [SMILES: CC(C)Oc1ccc(C2=NC(c3ccc(Cl)cc3)C(c3ccc(Cl)cc3)N2C(=O)N2CCNC(=O)C2)c(OC(C)C)c1]

### SJ-172550

In an embodiment, the MDM2 inhibitor is SJ-172550. SJ-172550 has the chemical structure and name shown as:

### methyl 2-[2-chloro-6-ethoxy-4-[(3-methyl-5-oxo-1-phenylpyrazol-4-ylidene)methyl]phenoxy]acetate

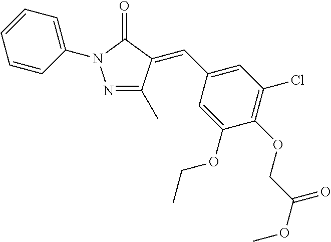 [SMILES: CCOc1cc(/C=C2/C(=O)N(c3ccccc3)N=C2C)cc(Cl)c1OCC(=O)OC]

### SAR405838 (MI-77301)

In an embodiment, the MDM2 inhibitor is SAR405838. SAR405838 has the chemical structure and name shown as:

### (2′R,3R,3'S,5'S)-6-chloro-3′-(3-chloro-2-fluorophenyl)-5′-(2,2-dimethylpropyl)-N-(4-hydroxycyclohexyl)-2-oxospiro[1H-indole-3,4′-pyrrolidine]-2′-carboxamide

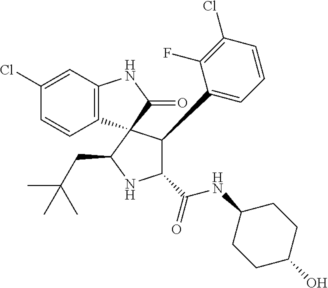 [SMILES: CC(C)(C)C[C@@H]1N[C@@H](C(=O)N[C@H]2CC[C@H](O)CC2)[C@H](c2cccc(Cl)c2F)[C@]12C(=O)Nc1cc(Cl)ccc12]

In an embodiment, the MDM2 inhibitor is MI-773. MI-773 has the chemical structure and name shown as:

### (2′R,3S,3'S,5′R)-6-chloro-3′-(3-chloro-2-fluorophenyl)-5′-(2,2-dimethylpropyl)-N-(4-hydroxycyclohexyl)-2-oxospiro[1H-indole-3,4′-pyrrolidine]-2′-carboxamide

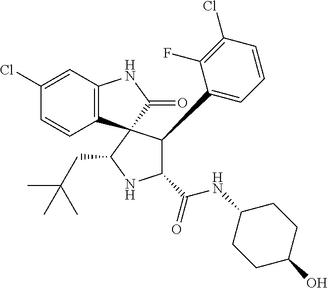 [SMILES: CC(C)(C)C[C@H]1N[C@@H](C(=O)N[C@H]2CC[C@H](O)CC2)[C@H](c2cccc(Cl)c2F)[C@@]12C(=O)Nc1cc(Cl)ccc12]

### MX69

In an embodiment, the MDM2 inhibitor is MX69. MX69 has the chemical structure and name shown as:

### 4-[8-[(3,4-dimethylphenyl)sulfamoyl]-3a,4,5,9b-tetrahydro-3H-cyclopenta[c]quinolin-4-yl]benzoic acid

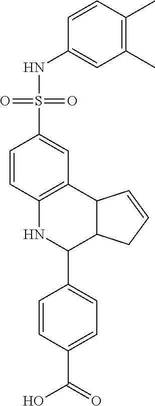 [SMILES: Cc1ccc(NS(=O)(=O)c2ccc3c(c2)C2C=CCC2C(c2ccc(C(=O)O)cc2)N3)cc1C]

### YH239-EE

In an embodiment, the MDM2 inhibitor is YH239-EE. YH239-EE has the chemical structure and name shown as:

### ethyl 3-[2-(tert-butylamino)-1-[(4-chlorophenyl)methyl-formylamino]-2-oxoethyl]-6-chloro-1H-indole-2-carboxylate

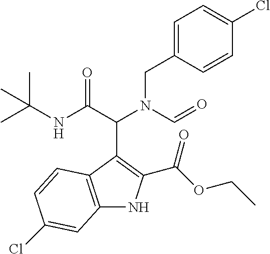 [SMILES: CCOC(=O)c1[nH]c2cc(Cl)ccc2c1C(C(=O)NC(C)(C)C)N(C=O)Cc1ccc(Cl)cc1]

### RO8994

In an embodiment, the MDM2 inhibitor is R08994. R08994 has the chemical structure and name shown as:

### (2′R,3R,3'S,5'S)—N-(4-carbamoyl-2-methoxyphenyl)-6-chloro-3′-(3-chloro-2-fluorophenyl)-5′-(2,2-dimethylpropyl)-2-oxospiro[1H-indole-3,4′-pyrrolidine]-2′-carboxamide

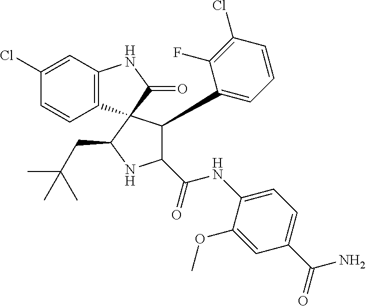 [SMILES: COc1cc(C(N)=O)ccc1NC(=O)C1N[C@@H](CC(C)(C)C)[C@@]2(C(=O)Nc3cc(Cl)ccc32)[C@H]1c1cccc(Cl)c1F]

### Nutlin-3b

In an embodiment, the MDM2 inhibitor is nutlin-3b. Nutlin-3b has the chemical structure and name shown as:

### 4-[(4R,5S)-4,5-bis(4-chlorophenyl)-2-(4-methoxy-2-propan-2-yloxyphenyl)-4,5-dihydroimidazole-1-carbonyl]piperazin-2-one

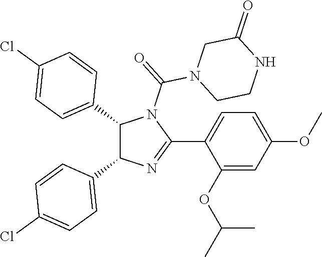 [SMILES: COc1ccc(C2=N[C@H](c3ccc(Cl)cc3)[C@H](c3ccc(Cl)cc3)N2C(=O)N2CCNC(=O)C2)c(OC(C)C)c1]

### Serdemetan (JNJ-26854165)

In an embodiment, the MDM2 inhibitor is Serdemetan. Serdemetan has the chemical structure and name shown as:

### 1-N-[2-(1H-indol-3-yl)ethyl]-4-N-pyridin-4-ylbenzene-1,4-diamine

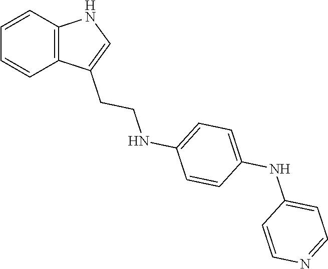 [SMILES: c1ccc2c(CCNc3ccc(Nc4ccncc4)cc3)c[nH]c2c1]

### NSC59984

In an embodiment, the MDM2 inhibitor is NSC59984. NSC59984 has the chemical structure and name shown as:

### (E)-1-(4-methylpiperazin-1-yl)-3-(5-nitrofuran-2-yl)prop-2-en-1-one

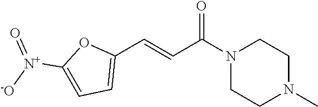

### CHEMBL2386350

In an embodiment, the MDM2 inhibitor is CHEMBL2386350. CHEMBL2386350 has the chemical structure and name shown as:

### 2-[4-[(4S,5R)-2-(4-tert-butyl-2-ethoxyphenyl)-4,5-bis(4-chlorophenyl)-4,5-dimethylimidazole-1-carbonyl]piperazin-1-yl]-1-morpholin-4-ylethanone

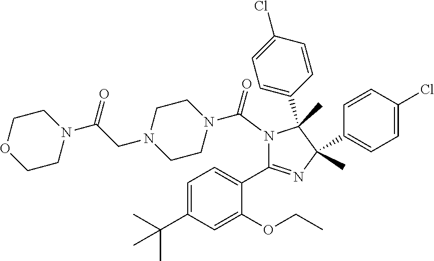 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CC(=O)N2CCOCC2)CC1]

### CGM0970B

In an embodiment, the MDM2 inhibitor is CGM0970B. CGM0970B has the chemical structure and name shown as:

### (1R)-1-(4-chlorophenyl)-6-methoxy-2-[4-[methyl-[[4-(4-methyl-3-oxopiperazin-1-yl)cyclohexyl]methyl]amino]phenyl]-7-propan-2-yloxy-1,4-dihydroisoquinolin-3-one

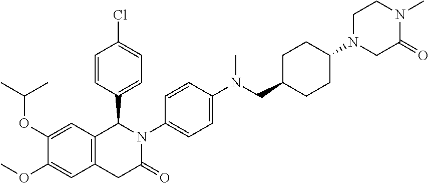 [SMILES: COc1cc2c(cc1OC(C)C)[C@@H](c1ccc(Cl)cc1)N(c1ccc(N(C)C[C@H]3CC[C@H](N4CCN(C)C(=O)C4)CC3)cc1)C(=O)C2]

### MK-8242

In an embodiment, the MDM2 inhibitor is MK-8242. MK-8242 has the chemical structure and name shown as:

### 4-amino-1-[(2R,3S,4S,5R)-3,4-dihydroxy-5-(hydroxymethyl)oxolan-2-yl]pyrimidin-2-one

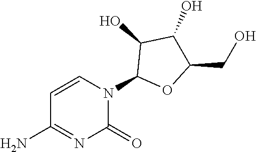 [SMILES: Nc1ccn([C@@H]2O[C@H](CO)[C@@H](O)[C@@H]2O)c(=O)n1]

### DS-3032

In an embodiment, the MDM2 inhibitor is DS-3032. DS-3032 has the chemical structure and name shown as:

### (3′R,4'S,5′R)—N-((3R,6S)-6-carbamoyltetrahydro-2H-pyran-3-yl)-6″-chloro-4′-(2-chloro-3-fluoropyridin-4-yl)-4,4-dimethyl-2″-oxodispiro[cyclohexane-1,2′-pyrrolidine-3′,3′″-indoline]-5′-carboxamide

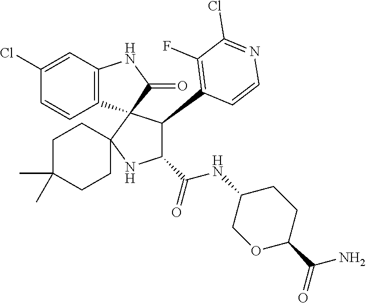 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

### DS-3032B

In an embodiment, the MDM2 inhibitor is DS-3032B. DS-3032B has the chemical structure and name shown as:

### (3′R,4'S,5′R)—N-((3R,6S)-6-carbamoyltetrahydro-2H-pyran-3-yl)-6″-chloro-4′-(2-chloro-3-fluoropyridin-4-yl)-4,4-dimethyl-2″-oxodispiro[cyclohexane-1,2′-pyrrolidine-3′,3′″-indoline]-5′-carboxamide 4-methylbenzenesulfonate

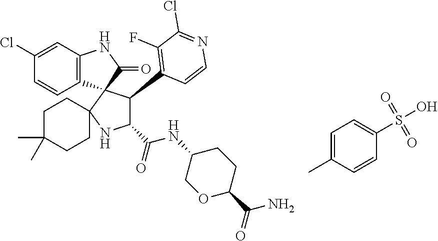 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21 ; Cc1ccc(S(=O)(=O)O)cc1]

### HDM201

In an embodiment, the MDM2 inhibitor is HDM201. HDM201 has the chemical structure and name shown as:

### (4S)-5-(5-chloro-1-methyl-2-oxopyridin-3-yl)-4-(4-chlorophenyl)-2-(2,4-dimethoxypyrimidin-5-yl)-3-propan-2-yl-4H-pyrrolo[3,4-d]imidazol-6-one

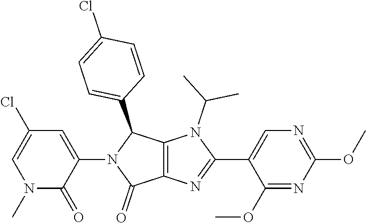 [SMILES: COc1ncc(-c2nc3c(n2C(C)C)[C@H](c2ccc(Cl)cc2)N(c2cc(Cl)cn(C)c2=O)C3=O)c(OC)n1]

### APG-115

In an embodiment, the MDM2 inhibitor is APG-115. APG-115 has the chemical structure and name shown as:

### 4-((3′R,4′S,5′R)-6″-Chloro-4′-(3-chloro-2-fluorophenyl)-1′-ethyl-2″-oxodispiro[cyclohexane-1,2′-pyrrolidine-3′,3″-indoline]-5′-carboxamido)bicyclo[2.2.2]octane-1-carboxylic Acid

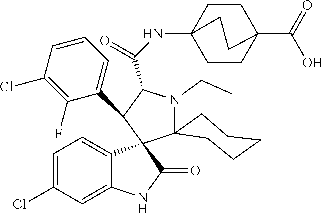 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

### MI-1061

In an embodiment, the MDM2 inhibitor is APG-115. APG-115 has the chemical structure and name shown as:

### 4-((3′R,4′S,5′R)-6″-chloro-4′-(3-chloro-2-fluorophenyl)-2″-oxodispiro[cyclohexane-1,2′-pyrrolidine-3′,3″-indoline]-5′-carboxamido)benzoic acid

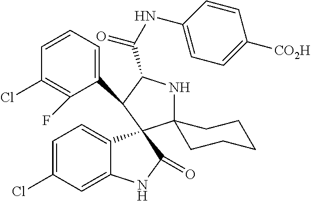 [SMILES: O=C(O)c1ccc(NC(=O)[C@@H]2NC3(CCCCC3)[C@@]3(C(=O)Nc4cc(Cl)ccc43)[C@H]2c2cccc(Cl)c2F)cc1]

### JAK Inhibitors

### Ruxolitinib

In an embodiment, the JAK inhibitor is Ruxolitinib (available from Incyte Corp. and Novartis AG). Ruxolitinib has the chemical structure and name shown as: (R)-3-(4-(7H-pyrrolo[2,3-d]pyrimidin-4-yl)-1H-pyrazol-1-yl)-3-cyclopentylpropanenitrile,

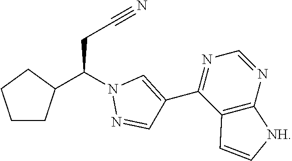 [SMILES: N#CC[C@H](C1CCCC1)n1cc(-c2ncnc3[nH]ccc23)cn1]

The preparation of this compound is described in U.S. Pat. Nos. 8,604,043, 7,834,022, 8,486,902, 8,530,485, 7,598,257, 8,541,425, and 8,410,265 the disclosures of which are incorporated by reference herein. In an embodiment, the JAK inhibitor is a compound selected from the structures disclosed in U.S. Pat. Nos. 8,604,043, 7,834,022, 8,486,902, 8,530,485, 7,598,257, 8,541,425, and 8,410,265, the disclosures of which are incorporated by reference herein.

In an embodiment, the JAK inhibitor is Ruxolitinib phosphate (available from Incyte Corp. and Novartis AG). In an embodiment, the JAK inhibitor is the phosphate salt of (R)-3-(4-(7H-pyrrolo[2,3-d]pyrimidin-4-yl)-1H-pyrazol-1-yl)-3-cyclopentylpropanenitrile.

### Baricitinib

In an embodiment, the JAK inhibitor is Baricitinib (available from Incyte Corp. and Eli Lilly & Co.). Baricitinib has the chemical structure and name shown as: 2-(3-(4-(7H-pyrrolo[2,3-d]pyrimidin-4-yl)-1H-pyrazol-1-yl)-1-(ethylsulfonyl)azetidin-3-yl)acetonitrile

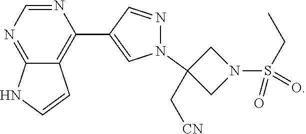 [SMILES: CCS(=O)(=O)N1CC(CC#N)(n2cc(-c3ncnc4[nH]ccc34)cn2)C1]

The preparation of this compound is described in U.S. Pat. Nos. 8,158,616 and 8,420,629, the disclosures of which are incorporated by reference herein. In an embodiment, the JAK inhibitor is a compound described in U.S. Pat. Nos. 8,158,616 and 8,420,629, the disclosures of which are incorporated by reference herein.

### Momelotinib

In an embodiment, the JAK inhibitor is Momelotinib (Gilead Sciences). Momelotinib is also known as CYT-387. Momelotinib has the chemical structure and name shown as: N-(cyanomethyl)-4-(2-((4-morpholinophenyl)amino)pyrimidin-4-yl)benzamide

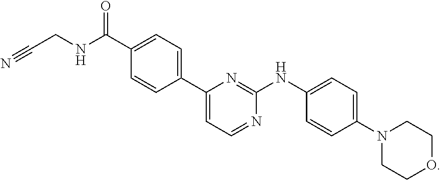 [SMILES: N#CCNC(=O)c1ccc(-c2ccnc(Nc3ccc(N4CCOCC4)cc3)n2)cc1]

The preparation of this compound is described in U.S. Pat. No. 8,486,941, the disclosure of which is incorporated by reference herein. In an embodiment, the JAK inhibitor is a compound described in U.S. Pat. No. 8,486,941, the disclosure of which is incorporated by reference herein.

### Ganetespib

In an embodiment, the JAK inhibitor is Ganetespib. Ganetespib has the chemical structure and name shown as: 5-(2,4-dihydroxy-5-isopropylphenyl)-4-(1-methyl-1H-indol-5-yl)-2,4-dihydro-3H-1,2,4-triazol-3-one

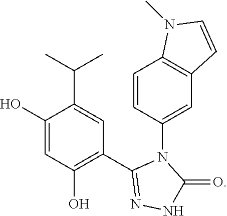 [SMILES: CC(C)c1cc(-c2n[nH]c(=O)n2-c2ccc3c(ccn3C)c2)c(O)cc1O]

The preparation of this compound is described in U.S. Pat. Nos. 7,825,148 and 8,628,752, the disclosures of which are incorporated by reference herein. In an embodiment, the JAK inhibitor is a compound described in U.S. Pat. Nos. 7,825,148 and 8,628,752, the disclosures of which are incorporated by reference herein.

### NS-018

In an embodiment, the JAK inhibitor is NS-018. NS-018 has the chemical structure and name shown as: (S)—N²-(1-(4-fluorophenyl)ethyl)-6-(1-methyl-1H-pyrazol-4-yl)-N⁴-(pyrazin-2-yl)pyrimidine-2,4-diamine

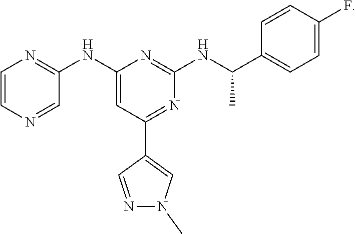 [SMILES: C[C@H](Nc1nc(Nc2cnccn2)cc(-c2cnn(C)c2)n1)c1ccc(F)cc1]

The preparation of this compound is described in U.S. Pat. Nos. 8,673,891 and 8,586,591, the disclosures of which are incorporated by reference herein. In an embodiment, the JAK inhibitor is a compound described in U.S. Pat. Nos. 8,673,891 and 8,586,591, the disclosures of which are incorporated by reference herein.

### BMS-911543

In an embodiment, the JAK inhibitor is BMS-911543. BMS-911543 has the chemical structure and name shown as: N,N-dicyclopropyl-4-((1,5-dimethyl-1H-pyrazol-3-yl)amino)-6-ethyl-1-methyl-1,6-dihydroimidazo[4,5-d]pyrrolo[2,3-b]pyridine-7-carboxamide

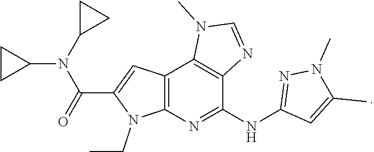 [SMILES: CCn1c(C(=O)N(C2CC2)C2CC2)cc2c3c(ncn3C)c(Nc3cc(C)n(C)n3)nc21]

The preparation of this compound is described in U.S. Pat. Nos. 8,673,933 and 8,202,881, the disclosures of which are incorporated by reference herein. In an embodiment, the JAK inhibitor is a compound described in U.S. Pat. Nos. 8,673,933 and 8,202,881, the disclosures of which are incorporated by reference herein.

### Gandotinib (LY2784544)

In an embodiment, the JAK inhibitor is Gandotinib. Gandotinib has the chemical structure and name shown as: 3-(4-chloro-2-fluorobenzyl)-2-methyl-N-(5-methyl-1H-pyrazol-3-yl)-8-(morpholinomethyl)imidazo[1,2-b]pyridazin-6-amine

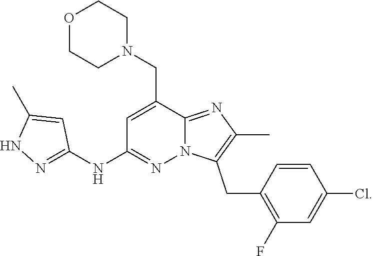 [SMILES: Cc1cc(Nc2cc(CN3CCOCC3)c3nc(C)c(Cc4ccc(Cl)cc4F)n3n2)n[nH]1]

The preparation of this compound is described in U.S. Pat. No. 7,897,600, the disclosure of which is incorporated by reference herein. In an embodiment, the JAK inhibitor is a compound described in U.S. Pat. No. 7,897,600, the disclosure of which is incorporated by reference herein.

### ENMD-2076

In an embodiment, the JAK inhibitor is ENMD-2076. ENMD-2076 has the chemical structure and name shown as: (E)-N-(5-methyl-1H-pyrazol-3-yl)-6-(4-methylpiperazin-1-yl)-2-styrylpyrimidin-4-amine

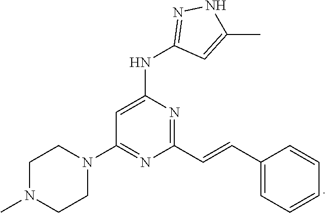 [SMILES: Cc1cc(Nc2cc(N3CCN(C)CC3)nc(/C=C/c3ccccc3)n2)n[nH]1]

The preparation of this compound is described in U.S. Pat. Nos. 8,153,630; 7,563,787 and 8,114,870, the disclosures of which are incorporated by reference herein. In an embodiment, the JAK inhibitor is a compound described in U.S. Pat. Nos. 8,153,630; 7,563,787 and 8,114,870, the disclosures of which are incorporated by reference herein.

### AT-9283

In an embodiment, the JAK inhibitor is AT-9283. AT-9283 has the chemical structure and name shown as: 1-cyclopropyl-3-(3-(5-(morpholinomethyl)-1H-benzo[d]imidazol-2-yl)-1H-pyrazol-4-yl)urea

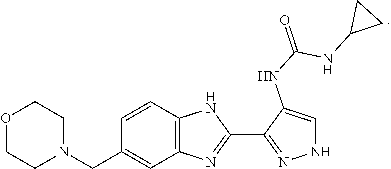 [SMILES: O=C(Nc1c[nH]nc1-c1nc2cc(CN3CCOCC3)ccc2[nH]1)NC1CC1]

The preparation of this compound is described in U.S. Pat. Nos. 8,399,442 and 7,977,477, the disclosures of which are incorporated by reference herein. In an embodiment, the JAK inhibitor is a compound described in U.S. Pat. Nos. 8,399,442 and 7,977,477, the disclosures of which are incorporated by reference herein.

### Pacritinib

In an embodiment, the JAK inhibitor is Pacritinib. Pacritinib has the chemical structure and name shown as: 11-(2-pyrrolidin-1-yl-ethoxy)-14,19-dioxa-5,7,26-triaza-tetracyclo[19.3.1.1(2,6).1(8,12)]heptacosa-1(25),2(26),3,5,8,10,12(27),16,21,23-decaene

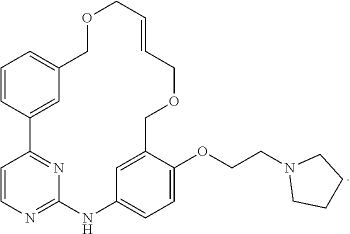 [SMILES: C1=C/COCc2cc(ccc2OCCN2CCCC2)Nc2nccc(n2)-c2cccc(c2)COC/1]

In an embodiment, the structure of Pacritinib may be a tautomeric form. The preparation of Pacritinib is described in U.S. Pat. Nos. 8,143,255; 8,153,632 and 8,415,338, the disclosures of which are incorporated by reference herein.

### AC-410

In an embodiment, the JAK inhibitor is AC-410 (available from Ambit Biosciences). AC-410 has the chemical structure and name shown as: (S)-(4-fluorophenyl)(4-((5-methyl-1H-pyrazol-3-yl)amino)quinazolin-2-yl)methanol

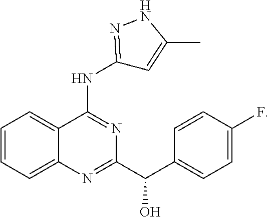 [SMILES: Cc1cc(Nc2nc([C@@H](O)c3ccc(F)cc3)nc3ccccc23)n[nH]1]

The preparation of racemic (4-fluorophenyl)(4-((5-methyl-1H-pyrazol-3-yl)amino)quinazolin-2-yl)methanol hydrochloride is described in Examples 3 and 12 of U.S. Pat. No. 8,349,851, the disclosure of which is incorporated by reference herein.

### AZD-1480

In an embodiment, the JAK inhibitor is AZD-1480. AZD-1480 has the chemical structure and name shown as: (S)-5-chloro-N²-(1-(5-fluoropyrimidin-2-yl)ethyl)-N⁴-(5-methyl-1H-pyrazol-3-yl)pyrimidine-2,4-diamine

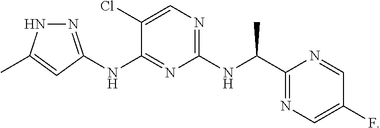 [SMILES: Cc1cc(Nc2nc(N[C@@H](C)c3ncc(F)cn3)ncc2Cl)n[nH]1]

The preparation of this compound is described in U.S. Pat. No. 8,088,784, the disclosure of which is incorporated by reference herein. In an embodiment, the JAK inhibitor is selected from the compounds described in U.S. Pat. No. 8,088,784, the disclosure of which is incorporated by reference herein.

### CYT387

In an embodiment, the JAK inhibitor is CYT387. CYT387 has the chemical structure and name shown as: N-(cyanomethyl)-4-(2-(4-morpholinophenylamino)pyrimidin-4-yl)benzamide

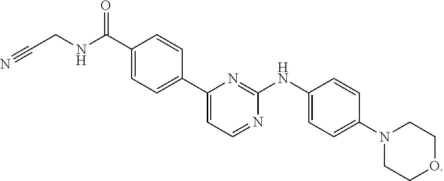 [SMILES: N#CCNC(=O)c1ccc(-c2ccnc(Nc3ccc(N4CCOCC4)cc3)n2)cc1]

The preparation of this compound is described in U.S. Pat. Nos. 9,809,559 and 8,486,941, the disclosures of which are incorporated by reference herein.

### TYK2-IN-2

In an embodiment, the JAK inhibitor is TYK2-IN-2. TYK2-IN-2 has the chemical structure and name shown as: 6-((3,5-dimethylphenyl)amino)-8-(methylamino)imidazo[1,2-b]pyridazine-3-carboxamide

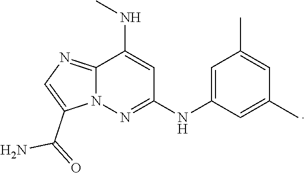 [SMILES: CNc1cc(Nc2cc(C)cc(C)c2)nn2c(C(N)=O)cnc12]

### SAR-20347

In an embodiment, the JAK inhibitor is SAR-20347. SAR-20347 has the chemical structure and name shown as: 2-(2-chloro-6-fluorophenyl)-5-[4-(morpholine-4-carbonyl)anilino]-1,3-oxazole-4-carboxamide

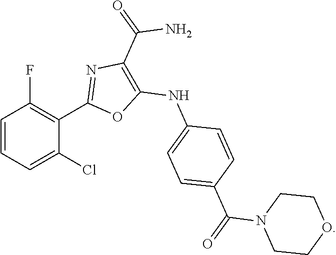 [SMILES: NC(=O)c1nc(-c2c(F)cccc2Cl)oc1Nc1ccc(C(=O)N2CCOCC2)cc1]

### Upadacitinib (ABT-494)

In an embodiment, the JAK inhibitor is Upadacitinib (ABT-494). Upadacitinib has the chemical structure and name shown as: (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide

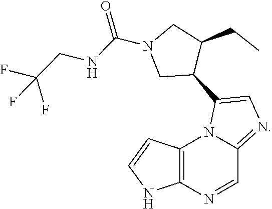 [SMILES: CC[C@@H]1CN(C(=O)NCC(F)(F)F)C[C@@H]1c1cnc2cnc3[nH]ccc3n12]

### WP1066

In an embodiment, the JAK inhibitor is WP1066. WP1066 has the chemical structure and name shown as: (E)-3-(6-bromopyridin-2-yl)-2-cyano-N-[(1S)-1-phenylethyl]prop-2-enamide

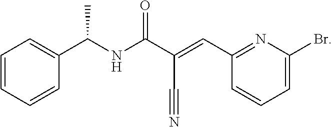 [SMILES: C[C@H](NC(=O)/C(C#N)=C/c1cccc(Br)n1)c1ccccc1]

### GLPG0634 (Filgotinib)

In an embodiment, the JAK inhibitor is GLPG0634 (Filgotinib). GLPG0634 has the chemical structure and name shown as: N-[5-[4-[(1,1-dioxo-1,4-thiazinan-4-yl)methyl]phenyl]-[1,2,4]triazolo[1,5-a]pyridin-2-yl]cyclopropanecarboxamide

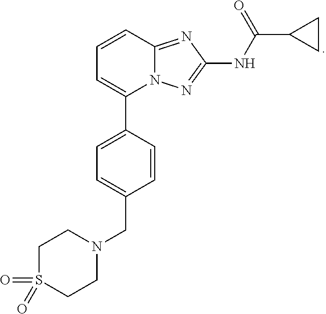 [SMILES: O=C(Nc1nc2cccc(-c3ccc(CN4CCS(=O)(=O)CC4)cc3)n2n1)C1CC1]

### TG101348 (Fedratinib; SAR 302503)

In an embodiment, the JAK inhibitor is TG101348 (Fedratinib; SAR 302503). TG101348 has the chemical structure and name shown as: N-tert-butyl-3-[[5-methyl-2-[4-(2-pyrrolidin-1-ylethoxy)anilino]pyrimidin-4-yl]amino]benzenesulfonamide

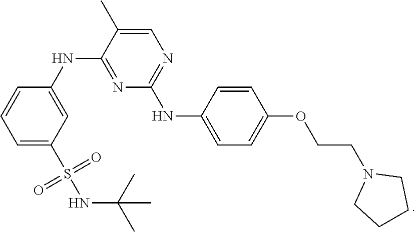 [SMILES: Cc1cnc(Nc2ccc(OCCN3CCCC3)cc2)nc1Nc1cccc(S(=O)(=O)NC(C)(C)C)c1]

### Cerdulatinib (PRT062070; PRT2070)

In an embodiment, the JAK inhibitor is Cerdulatinib (PRT062070; PRT2070). Cerdulatinib has the chemical structure and name shown as: 4-(cyclopropylamino)-2-[4-(4-ethylsulfonylpiperazin-1-yl)anilino]pyrimidine-5-carboxamide

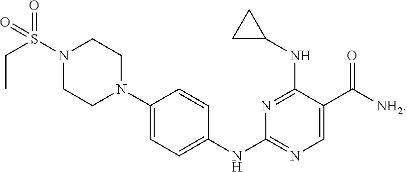 [SMILES: CCS(=O)(=O)N1CCN(c2ccc(Nc3ncc(C(N)=O)c(NC4CC4)n3)cc2)CC1]

### Tofacitinib

In an embodiment, the JAK inhibitor is Tofacitinib. Tofacitinib has the chemical structure and name shown as: 3-[(3R,4R)-4-methyl-3-[methyl(7H-pyrrolo[2,3-d]pyrimidin-4-yl)amino]piperidin-1-yl]-3-oxopropanenitrile

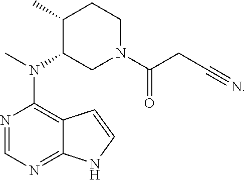 [SMILES: C[C@@H]1CCN(C(=O)CC#N)C[C@@H]1N(C)c1ncnc2[nH]ccc12]

### Itacitinib

In an embodiment, the JAK inhibitor is Itacitinib. Itacitinib has the chemical structure and name shown as: 2-[1-[1-[3-fluoro-2-(trifluoromethyl)pyridine-4-carbonyl]piperidin-4-yl]-3-[4-(7H-pyrrolo[2,3-d]pyrimidin-4-yl)pyrazol-1-yl]azetidin-3-yl]acetonitrile

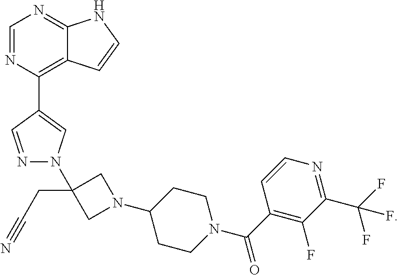 [SMILES: N#CCC1(n2cc(-c3ncnc4[nH]ccc34)cn2)CN(C2CCN(C(=O)c3ccnc(C(F)(F)F)c3F)CC2)C1]

### Decernotinib

In an embodiment, the JAK inhibitor is Decernotinib. Decernotinib has the chemical structure and name shown as: (2R)-2-methyl-2-[[2-(1H-pyrrolo[2,3-b]pyridin-3-yl)pyrimidin-4-yl]amino]-N-(2,2,2-trifluoroethyl)butanamide

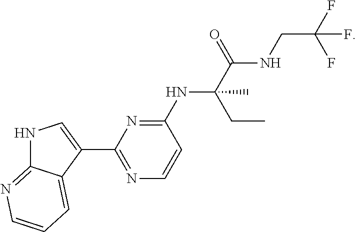 [SMILES: CC[C@@](C)(Nc1ccnc(-c2c[nH]c3ncccc23)n1)C(=O)NCC(F)(F)F]

### CHZ868

In an embodiment, the JAK inhibitor is CHZ868. CHZ868 has the chemical structure and name shown as: N-[4-[2-(2,4-difluoroanilino)-1,4-dimethylbenzimidazol-5-yl]oxypyridin-2-yl]acetamide

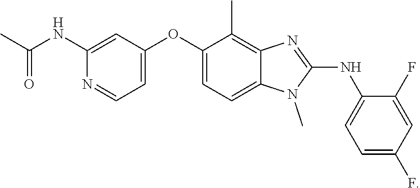 [SMILES: CC(=O)Nc1cc(Oc2ccc3c(nc(Nc4ccc(F)cc4F)n3C)c2C)ccn1]

### SB1317

In an embodiment, the JAK inhibitor is SB1317. SB1317 has the chemical structure and name shown as: (E)-6-methyl-12-oxa-3,6-diaza-2(4,2)-pyrimidina-1,4(1,3)-dibenzenacyclododecaphan-8-ene

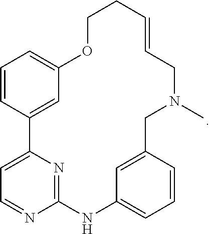 [SMILES: CN1C/C=C/CCOc2cccc(c2)-c2ccnc(n2)Nc2cccc(c2)C1]

### Solcitinib

In an embodiment, the JAK inhibitor is Solcitinib. Solcitinib has the chemical structure and name shown as: N-[5-[4-(3,3-dimethylazetidine-1-carbonyl)phenyl]-[1,2,4]triazolo[1,5-a]pyridin-2-yl]cyclopropanecarboxamide

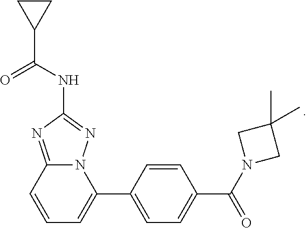 [SMILES: CC1(C)CN(C(=O)c2ccc(-c3cccc4nc(NC(=O)C5CC5)nn34)cc2)C1]

### Peficitinib

In an embodiment, the JAK inhibitor is Peficitinib. Peficitinib has the chemical structure and name shown as: 4-[[(1R,3S)-5-hydroxy-2-adamantyl]amino]-1H-pyrrolo[2,3-b]pyridine-5-carboxamide

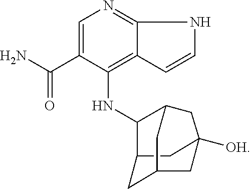 [SMILES: NC(=O)c1cnc2[nH]ccc2c1NC1C2CC3CC1CC(O)(C3)C2]

### CEP-33779

In an embodiment, the JAK inhibitor is CEP-33779. CEP-33779 has the chemical structure and name shown as: N-[3-(4-methylpiperazin-1-yl)phenyl]-8-(4-methylsulfonylphenyl)-[1,2,4]triazolo[1,5-a]pyridin-2-amine

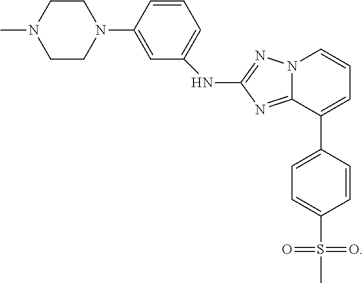 [SMILES: CN1CCN(c2cccc(Nc3nc4c(-c5ccc(S(C)(=O)=O)cc5)cccn4n3)c2)CC1]

### Pyridone 6

In an embodiment, the JAK inhibitor is Pyridone 6. Pyridone 6 has the chemical structure and name shown as: 2-(tert-butyl)-9-fluoro-3H-benzo[h]imidazo[4,5-f]isoquinolin-7-ol

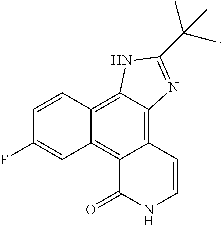 [SMILES: CC(C)(C)c1nc2c3cc[nH]c(=O)c3c3cc(F)ccc3c2[nH]1]

### LFM-A13

In an embodiment, the JAK inhibitor is LFM-A13. LFM-A13 has the chemical structure and name shown as: (Z)-2-cyano-N-(2,5-dibromophenyl)-3-hydroxybut-2-enamide

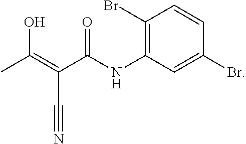 [SMILES: C/C(O)=C(\C#N)C(=O)Nc1cc(Br)ccc1Br]

### BMS-911543

In an embodiment, the JAK inhibitor is BMS-911543. BMS-911543 has the chemical structure and name shown as: (Z)—N,N-dicyclopropyl-4-((1,5-dimethyl-1,2-dihydro-3H-pyrazol-3-ylidene)amino)-6-ethyl-1-methyl-1,6-dihydroimidazo[4,5-d]pyrrolo[2,3-b]pyridine-7-carboxamide

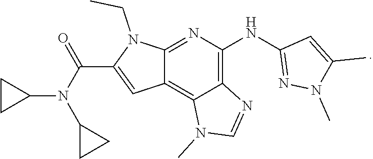 [SMILES: CCn1c(C(=O)N(C2CC2)C2CC2)cc2c3c(ncn3C)c(Nc3cc(C)n(C)n3)nc21]

### NS-018

In an embodiment, the JAK inhibitor is NS-018. NS-018 has the chemical structure and name shown as: 6-N-[(1S)-1-(4-fluorophenyl)ethyl]-4-(1-methylpyrazol-4-yl)-2-N-pyrazin-2-ylpyridine-2,6-diamine

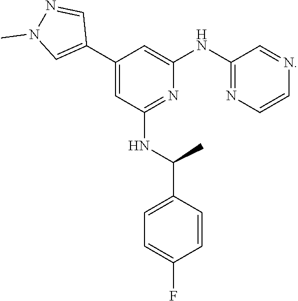 [SMILES: C[C@H](Nc1cc(-c2cnn(C)c2)cc(Nc2cnccn2)n1)c1ccc(F)cc1]

### JANEX-1

In an embodiment, the JAK inhibitor is JANEX-1. JANEX-1 has the chemical structure and name shown as: 4-[(6,7-dimethoxyquinazolin-4-yl)amino]phenol

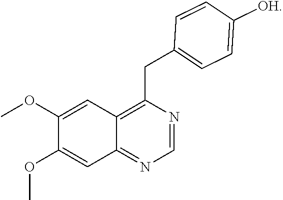 [SMILES: COc1cc2ncnc(Cc3ccc(O)cc3)c2cc1OC]

### TG101209

In an embodiment, the JAK inhibitor is TG101209. TG101209 has the chemical structure and name shown as: N-tert-butyl-3-[[5-methyl-2-[4-(4-methylpiperazin-1-yl)anilino]pyrimidin-4-yl]amino]benzenesulfonamide

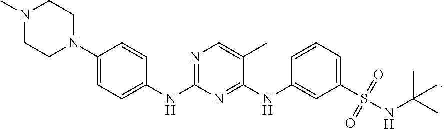 [SMILES: Cc1cnc(Nc2ccc(N3CCN(C)CC3)cc2)nc1Nc1cccc(S(=O)(=O)NC(C)(C)C)c1]

### WHI-P154

In an embodiment, the JAK inhibitor is WHI-P154. WHI-P154 has the chemical structure and name shown as: 2-bromo-4-[(6,7-dimethoxyquinazolin-4-yl)amino]phenol

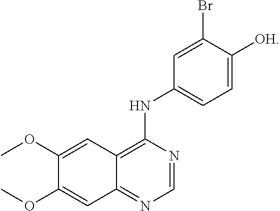 [SMILES: COc1cc2ncnc(Nc3ccc(O)c(Br)c3)c2cc1OC]

### NVP-BSK805

In an embodiment, the JAK inhibitor is NVP-BSK805. NVP-BSK805 has the chemical structure and name shown as: 4-[[2,6-difluoro-4-[3-(1-piperidin-4-ylpyrazol-4-yl)quinoxalin-5-yl]phenyl]methyl]morpholine

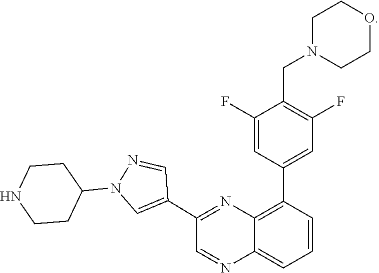 [SMILES: Fc1cc(-c2cccc3ncc(-c4cnn(C5CCNCC5)c4)nc23)cc(F)c1CN1CCOCC1]

### ZM39923

In an embodiment, the JAK inhibitor is ZM39923. ZM39923 has the chemical structure and name shown as: 3-[benzyl(propan-2-yl)amino]-1-naphthalen-2-ylpropan-1-one

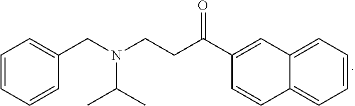 [SMILES: CC(C)N(CCC(=O)c1ccc2ccccc2c1)Cc1ccccc1]

### Ruxolitinib-S

In an embodiment, the JAK inhibitor is Ruxolitinib-S. Ruxolitinib-S has the chemical structure and name shown as: (3S)-3-cyclopentyl-3-[4-(7H-pyrrolo[2,3-d]pyrimidin-4-yl)pyrazol-1-yl]propanenitrile

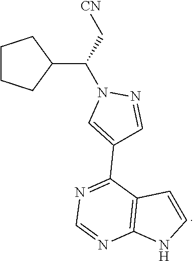 [SMILES: N#CC[C@@H](C1CCCC1)n1cc(-c2ncnc3[nH]ccc23)cn1]

### XL019

In an embodiment, the JAK inhibitor is XL019. XL019 has the chemical structure and name shown as: (2S)—N-[4-[2-(4-morpholin-4-ylanilino)pyrimidin-4-yl]phenyl]pyrrolidine-2-carboxamide

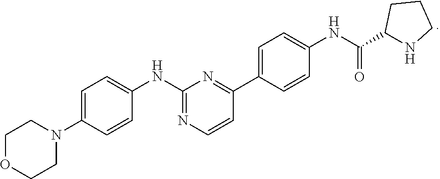

### AZ960

In an embodiment, the JAK inhibitor is AZ960. AZ960 has the chemical structure and name shown as: 5-fluoro-2-[[(1S)-1-(4-fluorophenyl)ethyl]amino]-6-[(5-methyl-1H-pyrazol-3-yl)amino]pyridine-3-carbonitrile

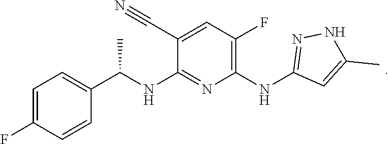 [SMILES: Cc1cc(Nc2nc(N[C@@H](C)c3ccc(F)cc3)c(C#N)cc2F)n[nH]1]

### JAK3-IN-1

In an embodiment, the JAK inhibitor is JAK3-IN-1. JAK3-IN-1 has the chemical structure and name shown as: N-[3-[[[5-chloro-2-[2-methoxy-4-(4-methylpiperazin-1-yl)anilino]pyrimidin-4-yl]amino]methyl]phenyl]prop-2-enamide

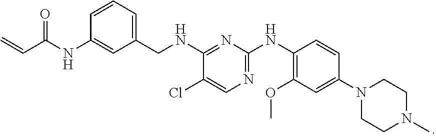 [SMILES: C=CC(=O)Nc1cccc(CNc2nc(Nc3ccc(N4CCN(C)CC4)cc3OC)ncc2Cl)c1]

### WHI-P97

In an embodiment, the JAK inhibitor is WHI-P97. WHI-P97 has the chemical structure and name shown as: 2,6-dibromo-4-[(6,7-dimethoxyquinazolin-4-yl)amino]phenol

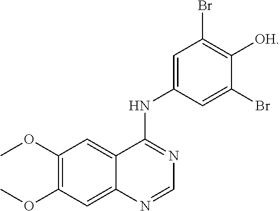 [SMILES: COc1cc2ncnc(Nc3cc(Br)c(O)c(Br)c3)c2cc1OC]

### RGB-286638

In an embodiment, the JAK inhibitor is RGB-286638. RGB-286638 has the chemical structure and name shown as: 1-[3-[4-[[4-(2-methoxyethyl)piperazin-1-yl]methyl]phenyl]-4-oxo-1H-indeno[1,2-c]pyrazol-5-yl]-3-morpholin-4-ylurea; dihydrochloride

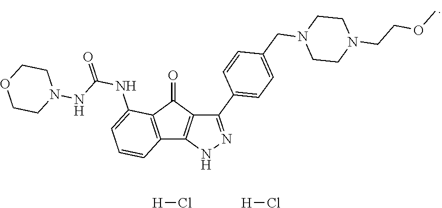 [SMILES: COCCN1CCN(Cc2ccc(-c3n[nH]c4c3C(=O)c3c(NC(=O)NN5CCOCC5)cccc3-4)cc2)CC1 ; Cl ; Cl]

### Tofacitinib(3R,4S)

In an embodiment, the JAK inhibitor is Tofacitinib(3R,4S). Tofacitinib(3R,4S) has the chemical structure and name shown as: 3-[(3R,4S)-4-methyl-3-[methyl(7H-pyrrolo[2,3-d]pyrimidin-4-yl)amino]piperidin-1-yl]-3-oxopropanenitrile

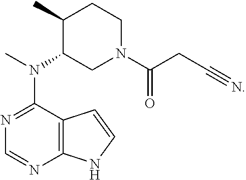 [SMILES: C[C@H]1CCN(C(=O)CC#N)C[C@@H]1N(C)c1ncnc2[nH]ccc12]

### NSC42834

In an embodiment, the JAK inhibitor is NSC42834. NSC42834 has the chemical structure and name shown as: 2-methyl-1-phenyl-4-pyridin-2-yl-2-(2-pyridin-2-ylethyl)butan-1-one

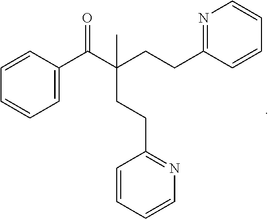 [SMILES: CC(CCc1ccccn1)(CCc1ccccn1)C(=O)c1ccccc1]

### PF-06651600

In an embodiment, the JAK inhibitor is PF-06651600. PF-06651600 has the chemical structure and name shown as: benzyl 2-(hydroxymethyl)-5-[(2-methylpropan-2-yl)oxycarbonylamino]piperidine-1-carboxylate

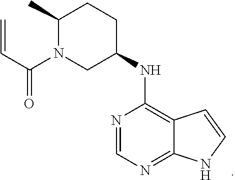 [SMILES: C=CC(=O)N1C[C@H](Nc2ncnc3[nH]ccc23)CC[C@@H]1C]

### Tofacitinib(3S,4S)

In an embodiment, the JAK inhibitor is Tofacitinib(3S,4S). Tofacitinib(3S,4S) has the chemical structure and name shown as: 3-[(3S,4S)-4-methyl-3-[methyl(7H-pyrrolo[2,3-d]pyrimidin-4-yl)amino]piperidin-1-yl]-3-oxopropanenitrile

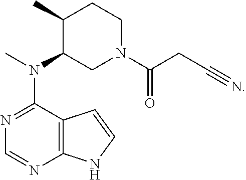 [SMILES: C[C@H]1CCN(C(=O)CC#N)C[C@H]1N(C)c1ncnc2[nH]ccc12]

### Tofacitinib(3S,4R)

In an embodiment, the JAK inhibitor is Tofacitinib(3S,4R). Tofacitinib(3S,4R) has the chemical structure and name shown as: 3-[(3S,4R)-4-methyl-3-[methyl(7H-pyrrolo[2,3-d]pyrimidin-4-yl)amino]piperidin-1-yl]-3-oxopropanenitrile

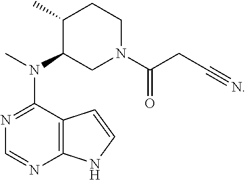 [SMILES: C[C@@H]1CCN(C(=O)CC#N)C[C@H]1N(C)c1ncnc2[nH]ccc12]

### AEG3482

In an embodiment, the JAK inhibitor is AEG3482. AEG3482 has the chemical structure and name shown as: 6-phenylimidazo[2,1-b][1,3,4]thiadiazole-2-sulfonamide

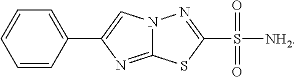 [SMILES: NS(=O)(=O)c1nn2cc(-c3ccccc3)nc2s1]

### Lestaurtinib (CEP-701)

In an embodiment, the JAK inhibitor is Lestaurtinib (CEP-701). Lestaurtinib has the chemical structure and name shown as: (5R,7S,8S)-7-hydroxy-7-(hydroxymethyl)-8-methyl-5,6,7,8,13,14-hexahydro-15H-16-oxa-4b,8a,14-triaza-5,8-methanodibenzo[b,h]cycloocta[jkl]cyclopenta[e]-as-indacen-15-one

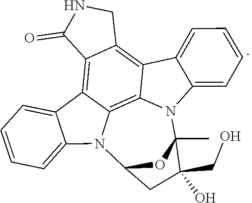 [SMILES: C[C@]12O[C@H](C[C@]1(O)CO)n1c3ccccc3c3c4c(c5c6ccccc6n2c5c31)CNC4=O]

### Oclacitinib

In an embodiment, the JAK inhibitor is Oclacitinib. Oclacitinib has the chemical structure and name shown as: N-methyl-1-[4-[methyl(7H-pyrrolo[2,3-d]pyrimidin-4-yl)amino]cyclohexyl]methanesulfonamide

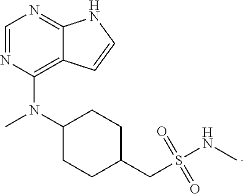 [SMILES: CNS(=O)(=O)CC1CCC(N(C)c2ncnc3[nH]ccc23)CC1]

In an embodiment, the JAK inhibitor is (E)-4-(2-(pyrrolidin-1-yl)ethoxy)-6,11-dioxa-3-aza-2(4,2)-pyrimidina-1(2,5)-furana-4(1,3)-benzenacyclododecaphan-8-ene. In an embodiment, the JAK inhibitor is (9E)-15-(2-(pyrrolidin-1-yl)ethoxy)-7,12,25-trioxa-19,21,24-triaza-tetracyclo[18.3.1.1(2,5).1(14,18)]hexacosa-1(24),2,4,9,14(26),15,17,20,22-nonaene. In an embodiment, the JAK inhibitor is a compound of Formula (LIV-A):

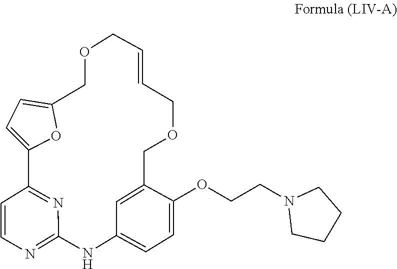 [SMILES: C1=C2COC/C=C/COCc3cc(ccc3OCCN3CCCC3)Nc3nccc(n3)C(=C1)O2]

or a pharmaceutically acceptable salt thereof. The preparation and properties of this JAK inhibitor are known to those of ordinary skill in the art, and for example are described in: Madan (2012) J. Immunol. 189, 4123-4134 and William (2012) J. Med. Chem. 55, 2623-2640.

In an embodiment, the JAK inhibitor is (R)-(4-fluorophenyl)(4-((5-methyl-1H-pyrazol-3-yl)amino)quinazolin-2-yl)methanol, which is also known in the art to be active as a JAK inhibitor. In an embodiment, the JAK inhibitor is racemic (4-fluorophenyl)(4-((5-methyl-1H-pyrazol-3-yl)amino)quinazolin-2-yl)methanol, which is also known in the art to be active as a JAK inhibitor.

In an embodiment, the JAK inhibitor is (S)-5-fluoro-2-((1-(4-fluorophenyl)ethyl)amino)-6-((5-methyl-1H-pyrazol-3-yl)amino)nicotinonitrile. In an embodiment, the JAK inhibitor is a compound of Formula (LX):

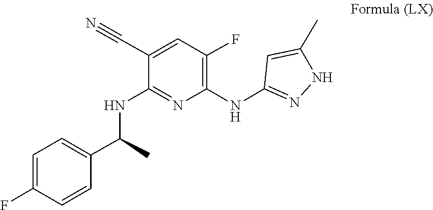 [SMILES: Cc1cc(Nc2nc(N[C@@H](C)c3ccc(F)cc3)c(C#N)cc2F)n[nH]1]

or a pharmaceutically acceptable salt thereof. The preparation of this compound is described in U.S. Pat. No. 8,324,252, the disclosure of which is incorporated by reference herein. In an embodiment, the JAK inhibitor is selected from the compounds described in U.S. Pat. No. 8,324,252, the disclosure of which is incorporated by reference herein.

In an embodiment, the JAK inhibitor is ((R)-7-(2-aminopyrimidin-5-yl)-1-((1-cyclopropyl-2,2,2-trifluoroethyl)amino)-5H-pyrido[4,3-b]indole-4-carboxamide, which is also named 7-(2-aminopyrimidin-5-yl)-1-{[(1R)-1-cyclopropyl-2,2,2-trifluoroethyl]amino}-5H-pyrido[4,3-b]indole-4-carboxamide. In an embodiment, the JAK inhibitor is a compound of Formula (LXII):

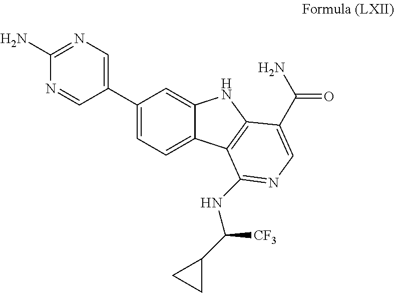 [SMILES: NC(=O)c1cnc(N[C@H](C2CC2)C(F)(F)F)c2c1[nH]c1cc(-c3cnc(N)nc3)ccc12]

or a pharmaceutically acceptable salt thereof. The preparation of this compound is known to those of ordinary skill in the art, and is described in Lim (2011) J. Med. Chem. 54, 7334-7349, the disclosure of which is incorporated by reference herein.

Further embodiments of the invention include the following:
1. Navtemadlin or a pharmaceutically acceptable salt thereof and ruxolitinib, for use in a method for treating myelofibrosis in a human subject who has been previously treated for myelofibrosis with ruxolitinib monotherapy and had a suboptimal response to said previous treatment with ruxolitinib monotherapy, the method comprising concurrent administration to the human of a therapeutically effective amount of the navtemadlin or a pharmaceutically acceptable salt thereof and the ruxolitinib.
2. Navtemadlin or a pharmaceutically acceptable salt thereof and ruxolitinib, for use according to embodiment 1, wherein the spleen volume reduction (SVR) in the human after the previous treatment with ruxolitinib monotherapy is <35% compared to the spleen volume prior to the previous treatment with ruxolitinib monotherapy; and the total symptom score (TSS) of the human after the previous treatment with ruxolitinib monotherapy is less than 50% compared to a TSS prior to the previous treatment with ruxolitinib monotherapy.
3. Navtemadlin or a pharmaceutically acceptable salt thereof and ruxolitinib, for use according to embodiment 1, wherein the spleen volume reduction (SVR) in the human after the previous treatment with ruxolitinib monotherapy is >zero but <35% compared to the spleen volume prior to the previous treatment with ruxolitinib monotherapy; and the total symptom score (TSS) of the human after the previous treatment with ruxolitinib monotherapy is >zero but less than 50% compared to a TSS prior to the previous treatment with ruxolitinib monotherapy.
4. Navtemadlin or a pharmaceutically acceptable salt thereof and ruxolitinib, for use according to any one of embodiments 1 or 3, wherein the spleen volume reduction (SVR) in the human after the previous treatment with ruxolitinib monotherapy is >zero but <20% compared to the spleen volume prior to the previous treatment with ruxolitinib monotherapy; and the total symptom score (TSS) of the human after the previous treatment with ruxolitinib monotherapy is >zero but less than 30% compared to a TSS prior to the previous treatment with ruxolitinib monotherapy.
5. Navtemadlin or a pharmaceutically acceptable salt thereof and ruxolitinib, for use according to any one of embodiments 1, 2 or 3, wherein the spleen volume reduction (SVR) in the human after the previous treatment with ruxolitinib monotherapy is ≥20% but <35% compared to the spleen volume prior to the previous treatment with ruxolitinib monotherapy; and the total symptom score (TSS) of the human after the previous treatment with ruxolitinib monotherapy is ≥30% but <50% compared to a TSS prior to the previous treatment with ruxolitinib monotherapy.
6. Navtemadlin or a pharmaceutically acceptable salt thereof and ruxolitinib, for use according to any one of embodiments 1-5, wherein the previous treatment with ruxolitinib monotherapy is about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, or about 52 weeks.
7. Navtemadlin or a pharmaceutically acceptable salt thereof and ruxolitinib, for use according to any one of embodiments 1-5, wherein the previous treatment with ruxolitinib monotherapy is at least 12 weeks.
8. Navtemadlin or a pharmaceutically acceptable salt thereof and ruxolitinib, for use according to any one of embodiments 1-5, wherein the previous treatment with ruxolitinib monotherapy is at least 18 weeks.
9. Navtemadlin or a pharmaceutically acceptable salt thereof and ruxolitinib, for use according to any one of embodiments 1-8, wherein the concurrent administration of navtemadlin and ruxolitinib is at least 12 weeks, at least 16 weeks, at least 18 weeks, at least 24 weeks, at least 28 weeks, at least 32 weeks, at least 36 weeks, at least 40 weeks, at least 44 weeks, at least 48 weeks, or at least 52 weeks.
10. Navtemadlin or a pharmaceutically acceptable salt thereof and ruxolitinib, for use according to any one of embodiments 1-9, wherein the concurrent administration of navtemadlin and ruxolitinib is at least 24 weeks.
11. Navtemadlin or a pharmaceutically acceptable salt thereof and ruxolitinib, for use according to any one of embodiments 1-10, wherein navtemadlin or a pharmaceutically acceptable salt thereof is administered at a dose of 240 mg daily.
12. Navtemadlin or a pharmaceutically acceptable salt thereof and ruxolitinib, for use according to any one of embodiments 1-11, wherein navtemadlin or a pharmaceutically acceptable salt thereof is administered on days 1-7 of a 28-day treatment cycle; wherein navtemadlin or a pharmaceutically acceptable salt thereof is not administered on days 8-28 of the 28-day treatment cycle.
13. Navtemadlin or a pharmaceutically acceptable salt thereof and ruxolitinib, for use according to embodiment 12, wherein navtemadlin or a pharmaceutically acceptable salt thereof is administered for one or more treatment cycles.
14. Navtemadlin or a pharmaceutically acceptable salt thereof and ruxolitinib, for use according to any one of embodiments 12 or 13, wherein navtemadlin or a pharmaceutically acceptable salt thereof is administered for one, two, three, four, five, six, seven, eight, nine, or ten treatment cycles.
15. Navtemadlin or a pharmaceutically acceptable salt thereof and ruxolitinib, for use according to any one of embodiments 13 or 14, wherein the spleen volume reduction (SVR) in the human after the one or more treatment cycles with navtemadlin is at least 25%, 30%, 35%, 40%, 45%, 50%, 55%, 60%, 65%, 70%, 75%, 80%, 85%, 90%, or 95% compared to the spleen volume prior to the one or more treatment cycles.
16. Navtemadlin or a pharmaceutically acceptable salt thereof and ruxolitinib, for use according to any one of embodiments 13-15, wherein the spleen volume reduction (SVR) in the human after the one or more treatment cycles with navtemadlin is at least 35%, 40%, 45%, 50%, 55%, 60%, 65%, 70%, 75%, 80%, 85%, 90%, or 95% compared to the spleen volume prior to the previous treatment with ruxolitinib monotherapy.
17. Navtemadlin or a pharmaceutically acceptable salt thereof and ruxolitinib, for use according to any one of embodiments 13-16, wherein the TSS in the human after the one or more treatment cycles with navtemadlin is reduced by at least 30%, 40%, 45%, 50%, 55%, 60%, 65%, 70%, 75%, 80%, 85%, 90%, or 95% compared to a TSS prior to the one or more treatment cycles.
18. Navtemadlin or a pharmaceutically acceptable salt thereof and ruxolitinib, for use according to any one of embodiments 13-17, wherein the TSS of the human after the one or more treatment cycles with navtemadlin is reduced by at least 50%, 55%, 60%, 65%, 70%, 75%, 80%, 85%, 90%, or 95% compared to a TSS prior to the previous treatment with ruxolitinib monotherapy.
19. Navtemadlin or a pharmaceutically acceptable salt thereof and ruxolitinib, for use according to any one of embodiments 13-18, wherein the SVR in the human after the one or more treatment cycles with navtemadlin is at least 25% compared to the spleen volume prior to the one or more treatment cycles and at least 35% compared to the spleen volume prior to the previous treatment with ruxolitinib monotherapy.
20. Navtemadlin or a pharmaceutically acceptable salt thereof and ruxolitinib, for use according to any one of embodiments 13-19, wherein the TSS of the human after the one or more treatment cycles with navtemadlin is reduced by at least 30% compared to a TSS prior to the one or more treatment cycles and at least 50% compared to the TSS prior to the previous treatment with ruxolitinib monotherapy.
21. Navtemadlin or a pharmaceutically acceptable salt thereof and ruxolitinib, for use according to any one of embodiments 13-20, wherein the SVR in the human after the one or more treatment cycles with navtemadlin is at least 25% compared to the spleen volume prior to the one or more treatment cycles and at least 35% compared to the spleen volume prior to the previous treatment with ruxolitinib monotherapy; wherein the TSS of the human after the one or more treatment cycles is reduced by at least 30% compared to a TSS prior to the one or more treatment cycles and at least 50% compared to the TSS prior to the previous treatment with ruxolitinib monotherapy.
22. Navtemadlin or a pharmaceutically acceptable salt thereof and ruxolitinib, for use according to any one of embodiments 1-21, wherein ruxolitinib is administered at a dose of 5 mg, 10 mg, 15 mg, 20 mg, or 25 mg twice daily during ruxolitinib monotherapy.
23. Navtemadlin or a pharmaceutically acceptable salt thereof and ruxolitinib, for use according to any one of embodiments 1-22, wherein ruxolitinib is administered at a dose of 5 mg, 10 mg, 15 mg, 20 mg, or 25 mg twice daily during concurrent administration.
24. Navtemadlin or a pharmaceutically acceptable salt thereof and ruxolitinib, for use according to any one of embodiments 1-23, wherein the human has a JAK2V617F mutation.
25. Navtemadlin or a pharmaceutically acceptable salt thereof and ruxolitinib, for use according to any one of embodiments 1-24, wherein the human does not have TP53 mutation.
26. Navtemadlin or a pharmaceutically acceptable salt thereof and ruxolitinib, for use according to any one of embodiments 1-25, wherein the human is JAK inhibitor-naïve prior to ruxolitinib monotherapy.

### EXAMPLES

### Example 1 Randomized Double-Blind Study Evaluating Safety and Efficacy of Navtemadlin Plus Ruxolitinib Vs. Placebo Plus Ruxolitinib in Myelofibrosis Patients Who have a Suboptimal Response to Ruxolitinib

This is a Phase 3, randomized, double-blind, placebo-controlled study of navtemadlin or placebo as add-on therapy to ruxolitinib in subjects with MF who have a suboptimal response after ≥18 weeks of treatment with ruxolitinib monotherapy.

Subjects with JAKi-naïve MF will be enrolled into a ruxolitinib monotherapy run-in period (FIG. 1). Those subjects that meet suboptimal response criteria and have TP53WT MF will be randomized in a double-blind, placebo-controlled method to receive navtemadlin or placebo as add-on therapy to ruxolitinib treatment. Each portion of the study design is described below.

Approximately 630 JAKi-naïve subjects with MF are expected to be enrolled to attain 180 randomized suboptimal response subjects (Harrison 2012; Verstovsek 2012; Mesa 2017). Subjects will receive ruxolitinib monotherapy at a dose ≥5 mg twice per day (BID) chosen at the discretion of the Investigator during the run-in period. After ≥18 weeks and <25 weeks of treatment, response to therapy will be assessed in subjects that have had a stable dose of ruxolitinib for ≥6 consecutive weeks (e.g., a dose ≥5 mg BID that has not required a treatment hold or dose adjustment). Subjects that have a suboptimal response to a stable dose of ruxolitinib monotherapy and meet eligibility criteria (including a stable dose of ruxolitinib ≥8 consecutive weeks) will be randomized in a double-blind, placebo-controlled method to receive navtemadlin or placebo as add-on therapy to ruxolitinib treatment. Note, a stable dose of ruxolitinib ≥8 consecutive weeks is set as a baseline for randomization; however, in some implementations, response assessment during the run-in period is allowed earlier, after a stable dose of ≥6 consecutive weeks.

Suboptimal response to ruxolitinib monotherapy is defined as >0% but <35% reduction in spleen volume by MRI/CT scan (central review) and >0% but <50% reduction in TSS by MFSAF v4.0. Subjects that do not meet criteria for randomization may continue receiving commercially available ruxolitinib as per standard of care treatment and will be discontinued from this study.

Approximately 180 evaluable subjects with a suboptimal response to ruxolitinib monotherapy will be randomized 2:1 to receive either navtemadlin as add-on therapy to ruxolitinib (N=120; Arm 1) or placebo as add-on therapy to ruxolitinib (N=60; Arm 2). Navtemadlin/placebo will be administered 240 mg once daily on Days 1-7 on a 28-day treatment cycle.

In both treatment arms, ruxolitinib will continue to be administered at the subject's stable dose (e.g., a dose ≥5 mg BID that did not require a treatment hold or dose adjustment in the 8 weeks prior to study treatment in the randomized period). For each subject, dose escalation or any dose increases of ruxolitinib during the randomized period above the stable dose are not allowed before the primary efficacy assessment.

A stratified, permuted-block randomization scheme will be used for treatment allocation, and will be based on one or more (e.g., at least 4) stratification factors:
SVR>0% but <20%,
SVR≥20% but <35%,
TSS reduction >0% but <30%,
TSS reduction ≥30% but <50%,
Optionally, International Prognostic Scoring System (IPSS; (Cervantes 2009) (Intermediate-1 vs Intermediate-2 or High), and/or
Optionally, total daily ruxolitinib dose at randomization (<20 mg vs ≥20 mg).

Radiological assessment of disease will be performed at specified timepoints.

The study will include a screening period, a run-in period, and for subjects who are eligible, a randomization period, and a follow-up period.

Screening period assessments will be performed within 28 days prior to initiation of the run-in period. Subjects who satisfy all eligibility criteria are eligible to enroll in the study.

The run-in period will follow subjects from the initiation of treatment on Cycle 1 Day 1 with ruxolitinib monotherapy until randomization to navtemadlin/placebo treatment as add-on therapy to ruxolitinib, or study discontinuation for subjects who are ineligible for participation in the randomized period of the study.

The randomization period consists of 28-day cycles and will extend from the first dose of add-on study treatment in the navtemadlin/placebo period of the study until the end of treatment (EOT) visit which should occur within 28 days from the last dose of study drug. Initiation of navtemadlin/placebo treatment should be performed as early as possible or within 3 days following randomization approval. In the randomized period, all subjects will continue study treatment until criteria for permanent discontinuation of study drug are met. These criteria include disease progression, or where the study drug is no longer tolerated by the subject, or study end. Efficacy evaluations will be performed, including but not limited to SVR25, TSS30, SVR35, and/or TSS50.

The follow-up period will begin once a subject discontinues study treatment in the randomized period of the study, and will continue until death, lost to follow-up, consent withdrawal, or study end, whichever occurs first. The follow-up period may include both response follow-up and long-term follow-up periods.

The response follow-up period will occur for randomized subjects who discontinue treatment for reasons other than disease progression and will include efficacy assessments at a minimum of every 12 weeks ±7 days until disease progression, withdrawal of consent, or start of subsequent anticancer therapy.

The long-term follow-up period will occur for randomized subjects with disease progression who will be followed for survival and subsequent anti-cancer therapy every 12 weeks ±14 days until study end.

### Example 2 Navtemadlin Combined with Ruxolitinib Synergistically Increase Apoptosis in a JAK2 V617F Cell Line

Apoptosis induced by navtemadlin, ruxolitinib, or the combination was assessed in the UKE-1 cell line, a TP53^{WT} JAK2 V617F cell line derived from a patient with essential thrombocytopenia that transformed into acute leukemia (Fiedler 2000). Addition of navtemadlin to ruxolitinib synergistically increased apoptosis (P<0.001) at 24 hours (data not shown). Quantitative comparison using Combenefit software of the combination versus single agent results using a highest single agent (HSA) model showed statistically significant synergy at clinically meaningful concentrations (Clevenger 2023).

### Example 3 Navtemadlin Combined with Ruxolitinib Synergize in Apoptosis of Chronic Phase MF Patient-Derived Progenitor Cells

Because the UKE-1 cell line does not fully represent myeloid cells in chronic phase myelofibrosis (MF cell lines cannot be used because they are TP53^{MUT}), the combination effect was confirmed in myeloid cells from MF patients. The effects of navtemadlin and ruxolitinib were assessed in apoptosis assays using peripheral blood mononuclear cells collected from patients with chronic phase MF. MF-cells were co-cultured with a stromal support layer to mimic the supportive signals more closely from the tumor microenvironment present in vivo.

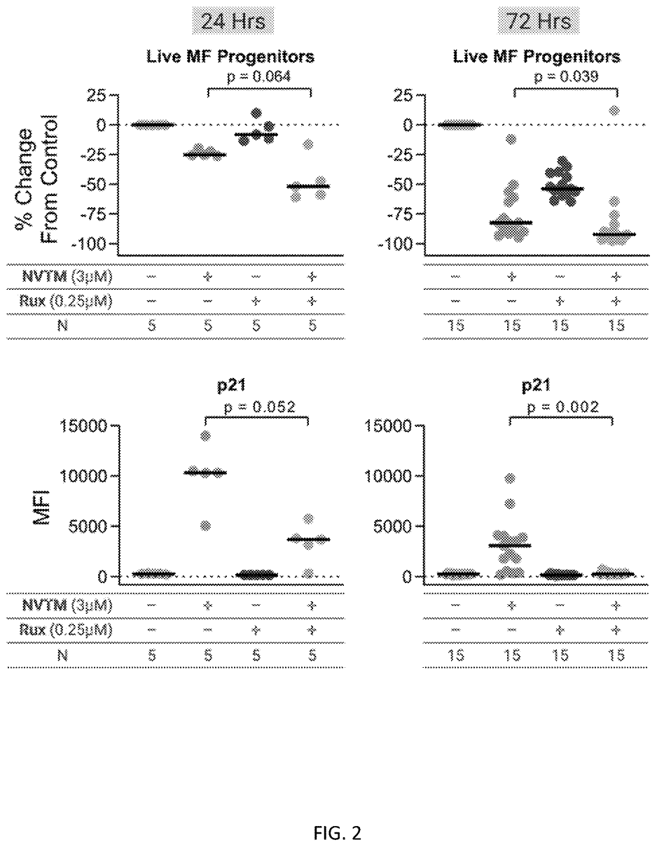

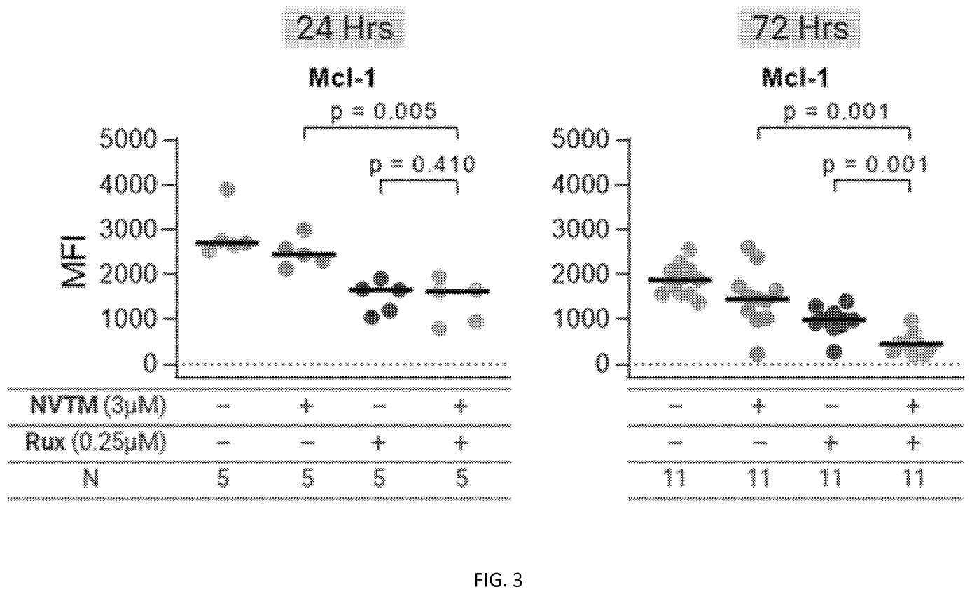

After 24 and 72 hours of co-culture, in the presence of clinically relevant concentrations of navtemadlin, ruxolitinib or the combination of the two drugs, progenitor cells were identified by flow cytometry (CD45^{+mid}, SSC^{low}, CD14⁻), and examined for markers of apoptosis (cleaved PARP). The combination of navtemadlin and ruxolitinib resulted in significantly more apoptosis of MF progenitor cells than from either drug alone at 72 hours (FIG. 2).

Activation of p53 also activates p21, a critical p53 induced cell cycle checkpoint that regulates cell cycle arrest and antagonizes apoptosis. As expected, navtemadlin alone markedly increased p21 expression in MF patient cells, whereas, as expected, ruxolitinib alone did not. Unexpectedly, the combination of navtemadlin and ruxolitinib significantly reduced levels of navtemadlin-induced p21 activation, evident as early as 24 hours after initial exposure. At 72 hours of combination exposure, p21 expression was nearly completely suppressed compared to navtemadlin alone (FIG. 2). These results demonstrated that MF patient cells treated with the combination of navtemadlin and ruxolitinib still induce p21 activation, but at a significantly lower apoptotic threshold needed to overcome the p21-mediated cell cycle arrest and apoptotic resistance. Surprisingly, in addition to the lowered apoptotic threshold of MF-cells, another synergistic mechanism of the combination was observed—significantly lowered expression of the BCL-2 family protein MCL-1, an important pro-survival, cell death escape pathway utilized by cancer cells in myeloid and B cell diseases (FIG. 2).

### Example 4 Cytotoxicity of Navtemadlin as Monotherapy or As Add-on Therapy to Ruxolitinib is Correlated with Spleen Volume Reduction

In MF, there is not a “true” cytotoxic target at the blast level to define clinical response, like in AML. However, ex vivo results demonstrate that navtemadlin robustly decreased circulating CD34⁺ cells (FIG. 4A and FIGS. 4B-C, respectively). Importantly, in both studies, reduction in CD34⁺ cells correlated with SVR—patients with the largest decreases in peripheral CD34⁺ cell count had the largest reductions in spleen volume.

In study KRT-232-101A, from baseline Cycle 1 Day 1 to Week 24, the median reduction in circulating CD34⁺ cells in subjects treated with 240 mg on Days 1-7 of a 28-day treatment cycle was 89.02% (range: −98.5% to 103.7%; (Vachhani 2021). Analysis of all evaluable subjects showed that change in CD34⁺ cell count was positively and significantly correlated with the magnitude of SVR (FIG. 4A).

In study KRT-232-109, the addition of navtemadlin to ongoing ruxolitinib treatment decreased CD34⁺ cell count at both Week 12 and Week 24, with a larger median reduction at Week 24 (FIG. 4B). Indeed, four out of five subjects with the largest CD34⁺ reductions from baseline Cycle 1 Day 1 to Week 24 were SVR35 responders (FIG. 4C).

These results provide compelling evidence that navtemadlin-induced cytotoxicity of malignant CD34⁺ cells improve the clinically important outcome of SVR and establishes the relevance of a cytotoxic mechanism for disease modifying outcomes in patients with MF. In addition, these results further support the relevance of MF-cell killing models demonstrating synergy in preclinical experiments (see, e.g., Examples 2 and 3). Importantly, this cytotoxicity-mediated mechanism is distinct from, and complementary to, the mechanisms through which ruxolitinib monotherapy acts (e.g., inhibition of the JAK-STAT signaling pathway).

### Example 5 KRT-232-114: Navtemadlin Monotherapy has Efficacy in Treatment-Naïve Myelofibrosis

KRT-232-114 was a Phase 2 open-label study in which the safety and efficacy of navtemadlin monotherapy was assessed in treatment-naïve patients with MF. A total of 14 subjects were treated with navtemadlin 240 mg on Days 1-7 on a 28-day cycle. Median treatment duration was 183.0 days (range, 7-539). A total of 14.3% of subjects (2/14) had SVR of ≥35% at the Week 24 assessment by central review, while 28.6% (4/14) had a TSS reduction of ≥50% by MFSAF v4.0 at the same time point.

### Example 6 KRT-232-IOA: Navtemadlin Monotherapy has Efficacy and Results in Disease-Modification in Ruxolitinib-Relapsed/Refractory Myelofibrosis

KRT-232-101A is a Phase 2 open-label, dose-finding study designed to evaluate the safety and efficacy of navtemadlin in subjects with MF who were relapsed or refractory to JAK inhibitor treatment. Subjects were treated with one of four dosing schedules to determine the optimal Phase 2 dose and schedule: navtemadlin 120 mg QD for Days 1-7 on a 21-cycle; navtemadlin 240 mg QD for Days 1-7 on a 21-day cycle; navtemadlin 240 mg QD for Days 1-7 on a 28-day cycle; or navtemadlin 240 mg QD for Days 1-5 on a 28-day cycle.

A total of 32 subjects were treated with navtemadlin 240 mg on Days 1-7 on a 28-day cycle. Fourteen (14) of these subjects had ruxolitinib washout of ≥28 days prior to the baseline imaging assessment; efficacy results are reported for this subset of patients to control for the effects on the spleen after ruxolitinib discontinuation prior to Cycle 1 Day 1. Median treatment duration for all 32 subjects was 175.5 days (range, 2-469). A total of 14.3% of subjects (2/14) had SVR of ≥35% at the Week 24 assessment by central review, while 14.3% (2/14) had a TSS reduction of ≥50% by MFSAF v4.0 at the same time point.

Navtemadlin Monotherapy Results in Disease-Modification of MF. Pharmacodynamic (PD) analysis showed disease modification with navtemadlin treatment, including reduced CD34⁺ cell count, and VAF reductions.

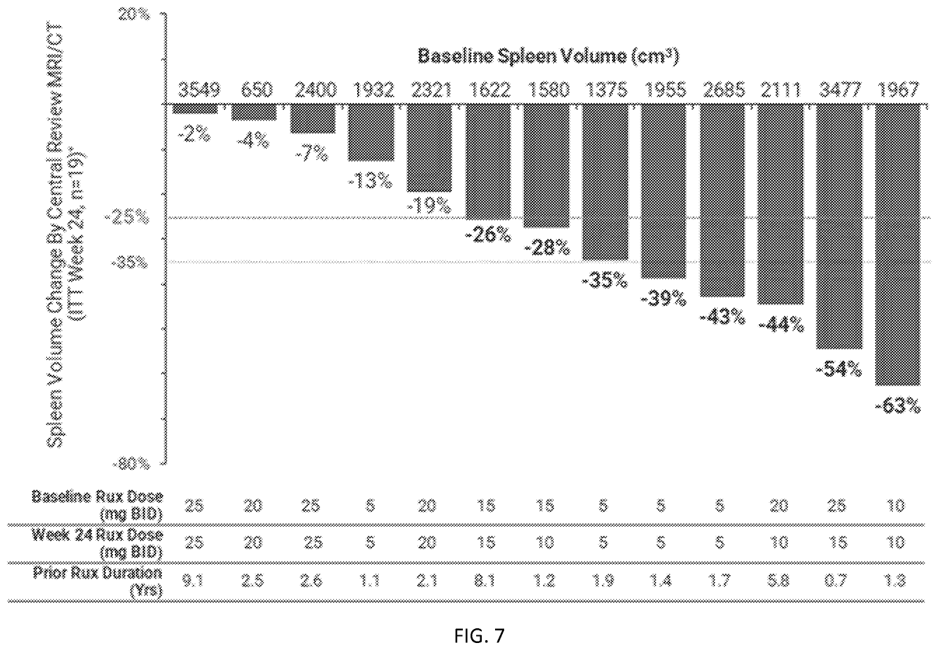

Reduction in CD34⁺ Cell Count: Reduction in CD34⁺ cell count was correlated with progression-free survival (PFS) and OS (FIG. 7) (Vachhani 2023). Subjects in which CD34⁺ cell count normalized (e.g., had <7 cells/μL), had significantly longer PFS and OS than subjects in which CD34⁺ cell counts remained elevated.

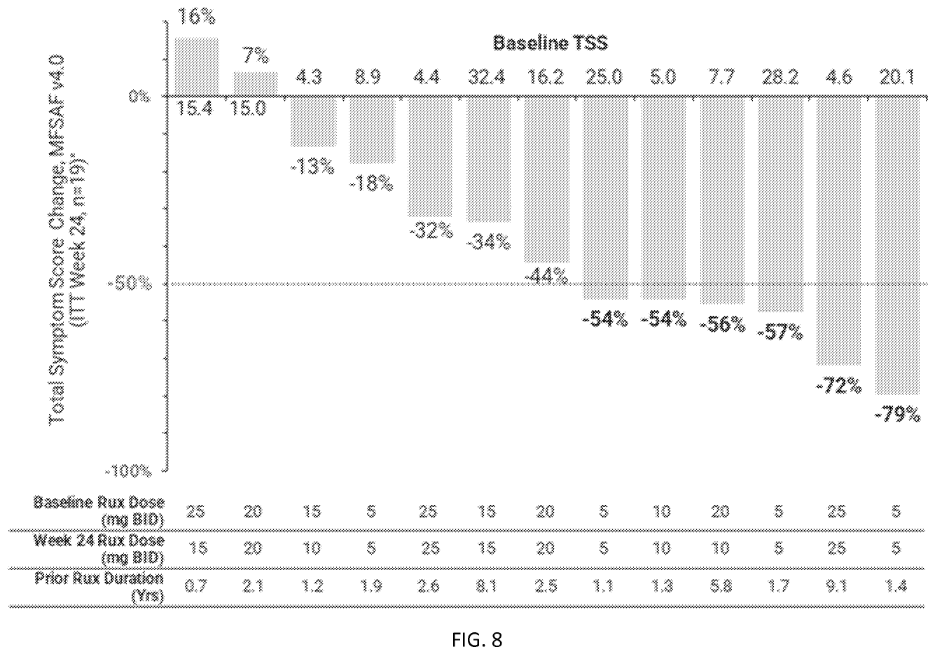

Reductions in Driver Gene VAF: Across all evaluable subjects, the magnitude of best VAF reduction was positively and significantly correlated (p<0.001) with the magnitude of best change in spleen volume. Similar to CD34⁺ count, reduction of VAF was correlated with PFS and OS (FIG. 8) (Vachhani 2023). Subjects with VAF reduction ≥20% had longer PFS and longer OS when compared with those with VAF reduction <20%.

Improvement in Bone Marrow Fibrosis: Across all evaluable subjects, 27.3% (12/44) demonstrated improvement in fibrosis grade from baseline to Week 24.

### Example 7 KRT-232-109: Addition of Navtemadlin to Ongoing Therapy with a Stable Dose of Ruxolitinib Results in Synergistic Efficacy Responses

KRT-232-109 is an ongoing, Phase 1/2 study of the safety and efficacy of navtemadlin as add-on therapy to ruxolitinib in MF patients with a suboptimal response to ruxolitinib monotherapy. Patients eligible for this study did not have a spleen response per IWG-MRT criteria and did not show signs of progression after prior treatment with a stable dose of ruxolitinib. In the dose escalation Phase 1 study, subjects were treated with navtemadlin 120 mg, 180 mg, or 240 mg on Days 1-7 on a 28-day cycle. In the Phase 2 dose expansion, additional subjects were treated at the Recommended Phase 2 Dose (RP2D) of 240 mg on Days 1-7 on a 28-day treatment cycle. As of 2 May 2023, a total of 28 subjects had been treated with the RP2D. Subjects had been treated with prior ruxolitinib monotherapy for a median duration of 21.6 months (range, 7-129) before the addition of navtemadlin. It is important to note that the ruxolitinib dose was not increased in any subjects after initiation of navtemadlin. Median treatment duration with navtemadlin as add-on therapy to an ongoing stable dose of ruxolitinib was 143 days (range, 3-449). Nineteen (19) subjects had reached Week 24 or discontinued prior to that time point and were evaluable for efficacy. A total of 31.6% of subjects (6/19) had SVR of ≥35% at the Week 24 assessment by central review (FIG. 7), while 31.6% (6/19) had a TSS reduction of ≥50% by MFSAF v4.0 at the same time point (FIG. 8).

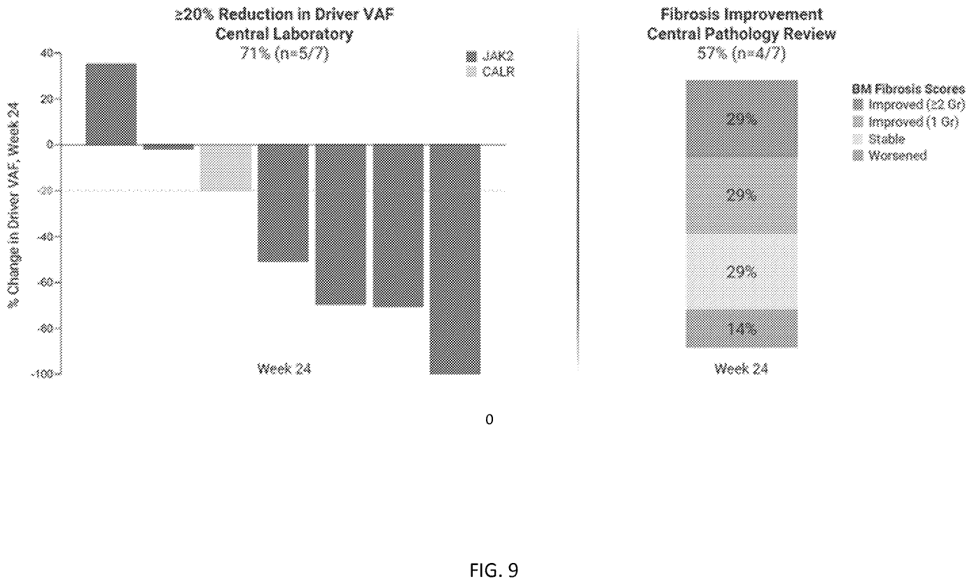

Navtemadlin Add-On Therapy to Ruxolitinib Results in Disease-Modification of MF. Navtemadlin add-on therapy to ruxolitinib reduced driver gene VAF and improved bone marrow fibrosis scores (FIG. 9). A total of 71% (5 of 7 evaluable subjects) had ≥20% reduction in driver VAF at Week 24, and 57% (4/7) had improvement in bone marrow fibrosis scores comparing baseline to Week 24. These preliminary results are a substantial improvement from changes observed at similar timepoints in ruxolitinib (Kvasnicka 2018), or navtemadlin monotherapy studies.

### Example 8 Clinical Evidence of Synergy with Addition of Navtemadlin to Ongoing Ruxolitinib

Clinical evidence of synergy resulting from the addition of navtemadlin to ruxolitinib demonstrated when comparing results from navtemadlin monotherapy studies KRT-232-114 and KRT-232-101A versus add-on study KRT-232-109 (Table 5). Study results demonstrated the efficacy of navtemadlin as add-on treatment to ongoing therapy with ruxolitinib was greater than efficacy achieved with navtemadlin alone, as measured by SVR and TSS reductions.

In studies KRT-232-114 and KRT-101A, patients with ruxolitinib-naïve or ruxolitinib relapsed/refractory MF were treated with navtemadlin monotherapy. In each of these studies, 14.3% of patients treated with navtemadlin monotherapy had a spleen response (SVR≥35%) at Week 24. In KRT-232-109, patients that did not have a spleen response per IWG-MRT criteria nor disease progression while on ruxolitinib monotherapy but on a stable dose of ruxolitinib for ≥8 weeks, (median treatment duration 21.6 months of ruxolitinib monotherapy), were treated with navtemadlin as add-on therapy to their ongoing ruxolitinib treatment. In this study, 31.6% of patients had SVR≥35% and TSS≥50% at Week 24, markedly better than responses reported in the navtemadlin monotherapy studies KRT-232-114 and KRT-232-101A.

TABLE 5
Clinical Evidence for Synergy of Navtemadlin and Ruxolitinib

| | KRT-232-114 | KRT-232-101A | KRT-232-109 |
|---|---|---|---|
| | (n = 14) | (n = 14) | (n = 19) |
| | Indication | | |
| | | Rux- | MF with |
| | | relapsed/ | suboptimal |
| | Rux-naïve | refractory | response to Rux |
| | MF | MF | monotherapy |
| | Treatment | | |
| | | | Navtemadlin |
| | | | added to an |
| | | | ongoing stable |
| | Navtemadlin | Navtemadlin | dose of |
| | monotherapy | monotherapy | ruxolitinib |
| SVR35 at Week 24 | 14.3% (2/14) | 14.3% (2/14) | 31.6% (6/19) |
| by Central Review | | | |
| TSS50 at Week 24 | 28.6% (4/14) | 14.3% (2/14) | 31.6% (6/19) |
| by MFSAF v4.0 | | | |
| SVR35 at Week 24 | 14.3% (2/14) | 14.3% (2/14) | 31.6% (6/19) |
| by Central Review | | | |
| TSS50 at Week 24 | 28.6% (4/14) | 14.3% (2/14) | 31.6% (6/19) |
| by MFSAF v4.0 | | | |

Abbreviations: Rux = ruxolitinib; SVR35 = spleen volume reduction of ≥35% at Week 24; TSS50 = total symptom score reduction of ≥50% at Week 24. Navtemadlin dose: All navtemadlin dosing was 240 mg on Days 1-7 of a 28-day cycle.

Of note, the novel SVR≥35% and TSS≥50% responses occurred in a patient population where ongoing ruxolitinib monotherapy did not provide further response benefit since SVR and TSS responses to ruxolitinib plateau after the initial 12 weeks of single agent ruxolitinib therapy (Verstovsek 2012). Thus, efficacy results observed in study KRT-232-109 were due to the addition of navtemadlin to ongoing ruxolitinib therapy. Together, these clinical results provide strong support for further development of navtemadlin as add-on therapy to ruxolitinib for patients with a suboptimal response to ruxolitinib monotherapy in study KRT-232-115. In addition, the magnitude of response after addition of navtemadlin to ongoing ruxolitinib (31.6% of patients with SVR≥35%) exceeded the magnitude observed in monotherapy trials (14.3% in frontline and in relapsed refractory settings) by ~2.2 fold, strongly suggestive of a synergistic effect of this drug combination.

The addition of navtemadlin to ruxolitinib treatment resulted in novel SVR≥35%; however, many other subjects had clinically relevant decreases in spleen volume of ≥25% to <35% (FIG. 7). In ruxolitinib monotherapy for the treatment of myelofibrosis, while numerically smaller than SVR≥35%, an SVR of ≥25% is associated with clinically meaningful increases in overall survival. Pooled analysis of results from COMFORT-1 and COMFORT-2 ruxolitinib monotherapy studies for the treatment of myelofibrosis reported that an SVR of ≥25% was associated with meaningful improvements in survival that were similar in magnitude to those associated with SVR≥35% (Vannucchi 2015). In the pooled analysis, the hazard ratio for survival in ruxolitinib-treated patients with SVR≥25% to <35% was 0.25 (95% CI: 0.11-0.61), representing a 75% reduction in risk of death versus patients that had SVR<10%. Most importantly, this hazard ratio was numerically similar to patients who achieved SVR≥35% to <50% (0.24 [95% CI: 0.11-0.56).

In study KRT-232-109, the addition of navtemadlin did not negatively affect ruxolitinib dosing. Dose reductions of ruxolitinib prior to Week 24 were rare, occurring in only 15.8% (3/19) of patients and all three of these patients achieved at least a 25% reduction in spleen volume at Week 24, providing clinical evidence that ruxolitinib dose reductions did not have a negative effect on patients' ability to achieve additional clinical benefit.

The safety profile of navtemadlin add-on therapy to ruxolitinib in study KRT-232-109 was acceptable. A total of 19.4% of subjects had a serious adverse event (SAE) and 8.3% of subjects had navtemadlin-related SAEs. Anemia was the only SAE that occurred in >1 subject (occurring in 2 subjects, 5.6%). Navtemadlin-related grade 3/4 TEAEs were reported in 44.4% of subjects. The most frequently reported navtemadlin-related Grade 3/4 TEAEs (>5%) were thrombocytopenia/platelet count decreased (25%), anemia (13.9%), nausea (11.1%), and diarrhea, neutrophil count decreased, and leukocytosis (5.6% each).

### Example 9 Study KRT-232-115: A Phase 3, Randomized, Double-blind, Add-on Study Evaluating the Safety and Efficacy of Navtemadlin Plus Ruxolitinib vs Placebo Plus Ruxolitinib in JAK Inhibitor-Naïve Patients with Myelofibrosis Who Have a Suboptimal Response to Ruxolitinib

The study includes a ruxolitinib monotherapy run-in period of >18 weeks but <25 weeks during which subjects will receive standard of care ruxolitinib treatment. After completion of the run-in period, eligible subjects identified as suboptimal responders to ruxolitinib monotherapy will be randomized 2:1 in a double-blind, placebo-controlled manner to receive either add-on navtemadlin (Arm 1) or add-on placebo (Arm 2) in the setting of continuous ruxolitinib use. All subjects assessed as refractory or optimally responding to ruxolitinib monotherapy will be discontinued from the study.

The run-in period will extend from the initiation of treatment with ruxolitinib monotherapy until randomization to navtemadlin/placebo as add-on therapy to ruxolitinib treatment (or study discontinuation for subjects who are ineligible for participation in the randomized period of the study). During the run-in period, eligible subjects with JAK inhibitor-naïve MF will receive ruxolitinib monotherapy at a dose 5 mg twice per day (BID) chosen at the discretion of the Investigator. Spleen volume and MF symptoms before and after 18 weeks but <25 weeks of ruxolitinib monotherapy will be assessed in subjects who have had a stable dose of ruxolitinib for 6 consecutive weeks (i.e., a dose 5 mg BID that did not require a treatment hold or dose adjustment). These assessments should be performed within 14 days prior to randomization.

The randomized period consists of 28-day treatment cycles and will extend from the first dose of add-on navtemadlin/placebo until the end of treatment (EOT) visit, which should occur within 28 days from the last dose of study drug. Approximately 180 subjects identified as suboptimal responders to ruxolitinib who meet eligibility criteria will be randomized (2:1) in a double-blind, placebo-controlled manner to receive either add-on navtemadlin (Arm 1; n=120) or add-on placebo (Arm 2; n=60) in the setting of continuous ruxolitinib treatment. Suboptimal response to ruxolitinib monotherapy is defined as >0% but <35% reduction in spleen volume by MRI/CT scan (central review) and >0% but <50% reduction in TSS by MFSAF v4.0. Key eligibility requirements are that subjects must be on a stable dose of ruxolitinib for ≥8 consecutive weeks (note that a response assessment during the monotherapy run-in period is allowed after a stable dose of ≥6 consecutive weeks) and have TPS3^{WT} MF. Subjects who do not meet the criteria for randomization will be discontinued from the study and may continue to receive commercially available ruxolitinib as per standard of care treatment.

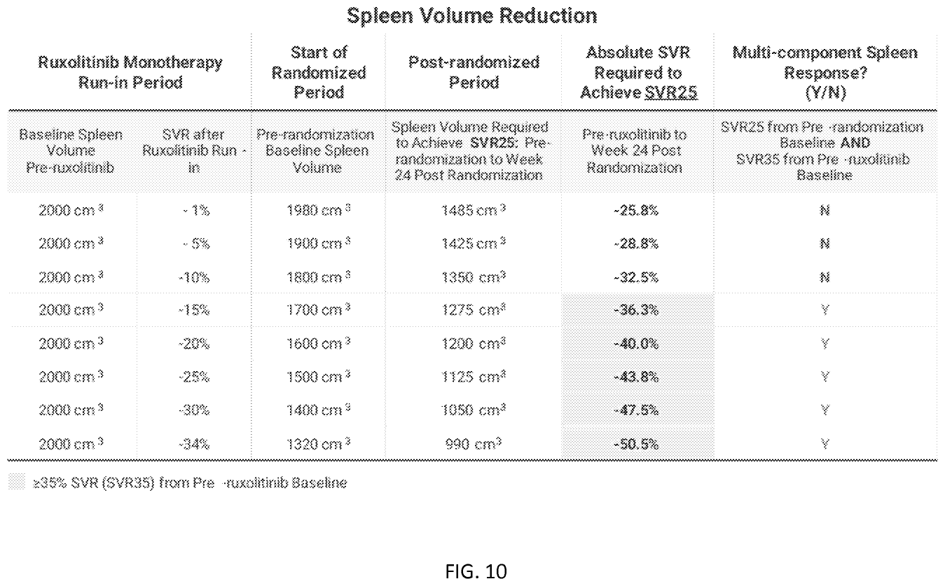

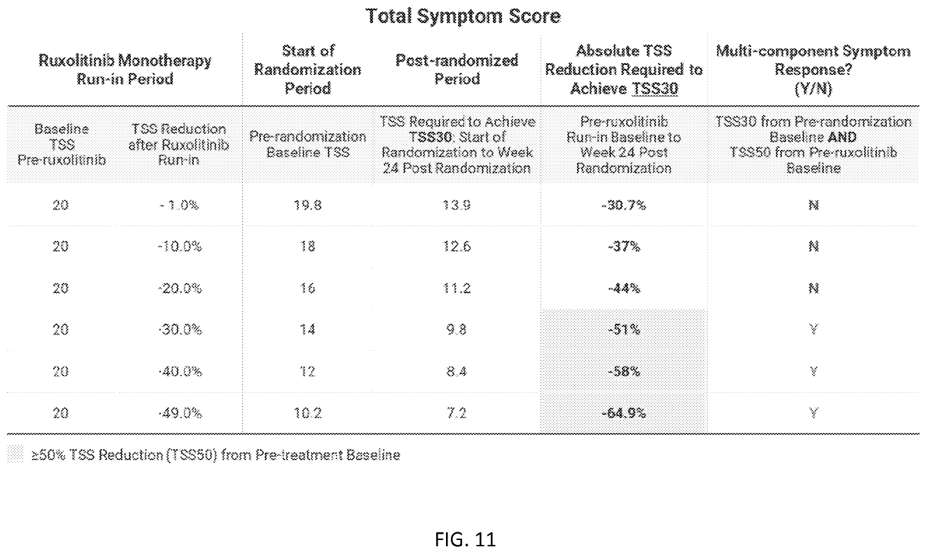

A study of a plurality of subjects will be performed to measure spleen size and symptoms for JAKi-naïve MF patients at three key time points: at a run-in period prior to treatment with ruxolitinib monotherapy treatment (pre-ruxolitinib baseline), at a second time point after ruxolitinib monotherapy treatment (the pre-randomization baseline), and at a third time point after add-on treatment (e.g., 24 weeks after randomization to either add-on navtemadlin or add-on placebo). Efficacy evaluations will be performed, including but not limited to SVR25, TSS30, SVR35, and/or TSS50. This unique study design allows the evaluation of the contribution of effect of either the combination of the first composition and the second composition or the combination of the first composition and the placebo (e.g., add-on navtemadlin or add-on placebo) by comparing a multi-component endpoint comprising two component endpoints SVR25 and TSS30 as determined based on a comparison of a second response set to a second set of baseline measurements (e.g., from a pre-randomization baseline) to the established SVR35 and TSS50 thresholds (e.g., from a pre-ruxolitinib baseline). The multi-component endpoints ensure that only patients who achieve an SVR35 or TSS50 in the first set of baseline measurements (e.g., from the pre-ruxolitinib baseline) are defined as responders if achieving both SVR25 and TSS30 in the second set of baseline measurements (e.g., from the pre-randomization baseline), thereby attributing within-patient clinically meaningful benefit according to established thresholds (FIG. 10, FIG. 11). In one preferred embodiment, the multi-component endpoints comprise: 1. the proportion of patients in each arm who achieve an SVR of ≥25% (SVR25) from the pre-randomization baseline and an SVR of ≥35% (SVR35) from the pre-ruxolitinib baseline; and 2. the proportion of patients in each arm who achieve a TSS reduction of ≥30% (TSS30) from the pre-randomization baseline and a TSS reduction of ≥50% (TSS50) from the pre-ruxolitinib baseline.

Multi-component spleen response endpoints based on spleen volume reduction are shown in FIG. 10. Patients achieving an SVR35 (spleen volume reduction ≥35%) from the pre-ruxolitinib baseline are defined as responders (despite achieving a SVR25 (spleen volume reduction ≥25%) from pre-randomization baseline). Multi-component spleen response endpoints based on spleen volume reduction are shown in FIG. 11. Patients achieving a TSS50 (≥50% total symptom score reduction) from the pre-ruxolitinib baseline are defined as responders (despite achieving a TSS30 (≥30% total symptom score reduction) from pre-randomization baseline).

Taken together, the multi-component endpoints will isolate the contribution of add-on navtemadlin, while ensuring the magnitude of spleen and symptom benefit is clinically meaningful from the established SVR35 and TSS50 endpoints perspective.

### Concordance Analysis of SVR25 and TSS30 to SVR35 and TSS50

To tie the response thresholds of SVR25 and TSS30, as measured from the pre-randomization baseline, with the historic thresholds for clinically meaningful benefit of SVR35 and TSS50 in the JAKi-naïve population (e.g., from the pre-ruxolitinib baseline), a three-step approach was taken.

### Step One:

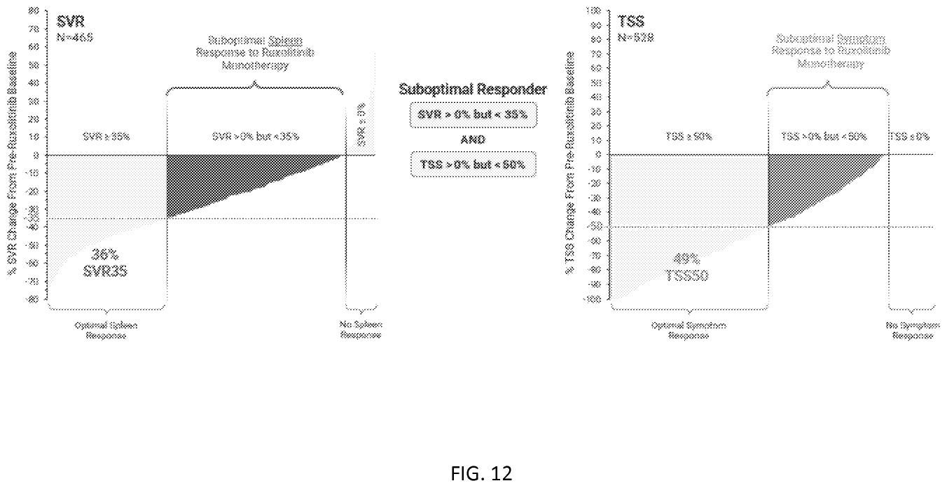

Data was pooled from published phase 3 studies for patients treated with ruxolitinib in the JAKi-naive MF setting (Verstovsek 2012; Mesa 2017; Pemmaraju 2023; Rampal 2023) to understand the natural history of patients with MF who achieve a suboptimal response to ruxolitinib, as defined in Study KRT-232-115 (e.g., a subject with an SVR>0% but <35% and a TSS reduction >0% but <50%) (FIG. 12).

FIG. 12 illustrates suboptimal spleen and symptom responders from the pooled phase 3 ruxolitinib data, including patients treated with ruxolitinib monotherapy with and without placebo. SVR data was obtained for 465 subjects from the three studies COMFORT-1, SIMPLIFY-1, and TRANSFORM-1 (Verstovsek 2012; Mesa 2017; Pemmaraju 2023). TSS data was obtained for 528 subjects from the three studies. Week-24 data used as a proxy to Week-18 suboptimal response assessment.

### Step Two:

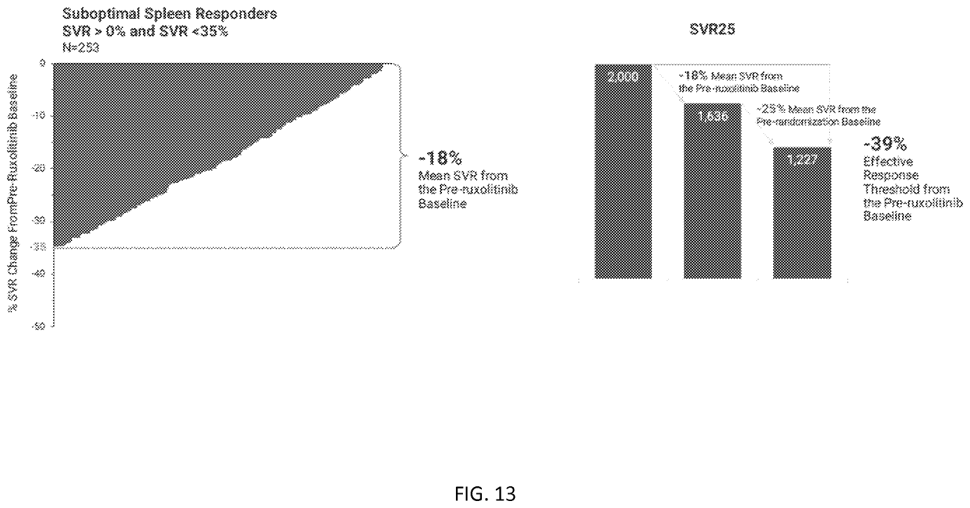

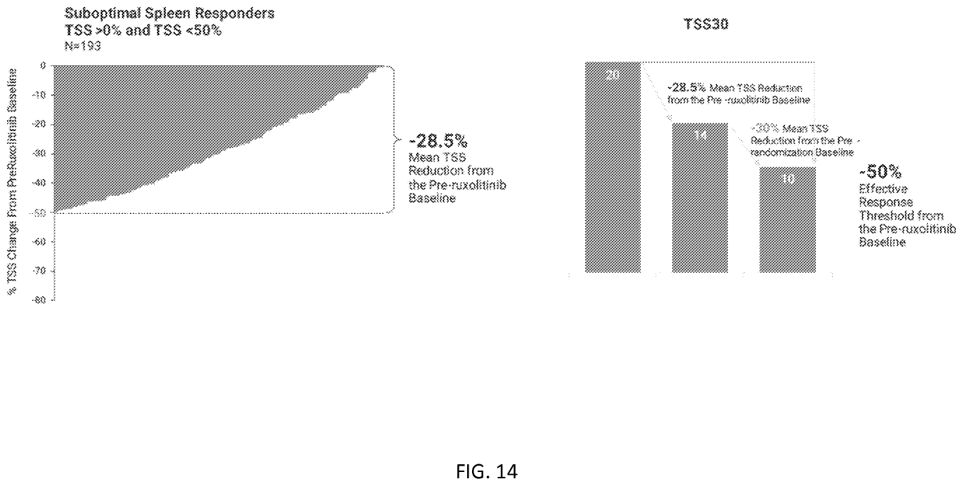

From the pooled dataset, it was determined that among the JAKi-naive MF patients with a suboptimal response to ruxolitinib the mean SVR was 18% (FIG. 13) and the mean TSS reduction was 28.5% (FIG. 14).

The information was then incorporated with the pre-randomization thresholds of SVR25 and TSS30 to determine congruence with historical thresholds of clinical benefit (e.g., SVR35 and TSS50) established in JAKi-naïve MF patients, as measured from the pre-ruxolitinib baseline.

An additional SVR of 25% to a mean SVR of 18% would result in a 39% effective response threshold from the pre-ruxolitinib baseline, more than the current SVR35 standard. Similarly, an additional TSS reduction of 30% to a mean TSS reduction of 28.5% would establish a 50% effective response threshold from the pre-ruxolitinib baseline, at parity with the current standard of TSS50.

### Step Three:

Lastly, a concordance analysis was performed comparing two sets of outcomes: SVR25 and TSS30 (measured from the pre-randomization baseline) with SVR35 and TSS50 (measured from the pre-ruxolitinib baseline). This was done to understand how well these outcomes align when assessing the effect of add-on navtemadlin or add-on placebo in suboptimal responders in study KRT-232-115.

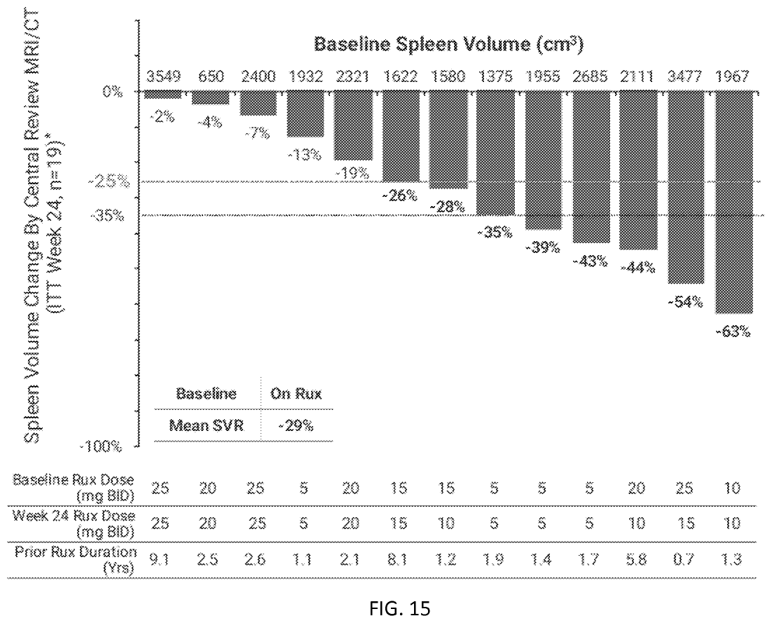

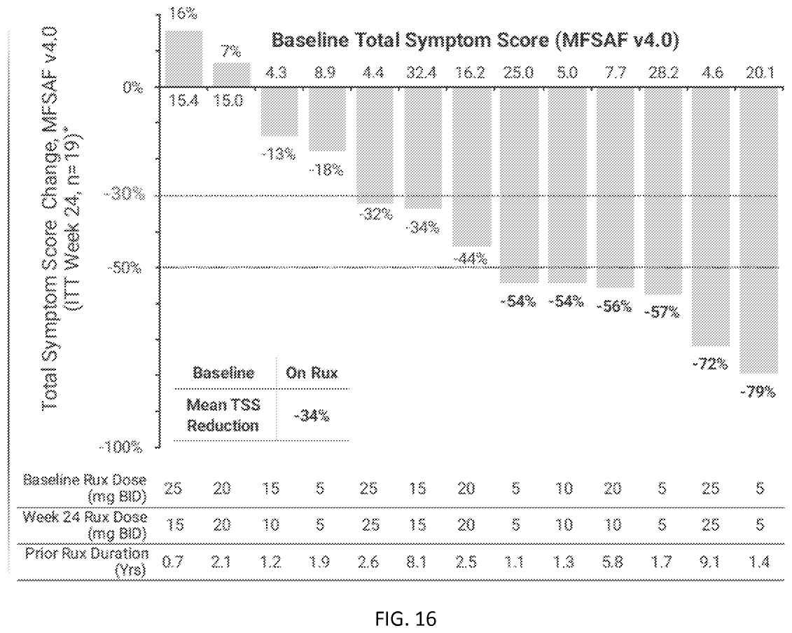

To do this, suboptimal responder patient data was pooled from the published phase 3 studies in JAKi-naïve MF (Verstovsek 2012; Mesa 2017; Pemmaraju 2023; Rampal 2023), together with the results from KRT-232-109 (FIG. 15, FIG. 16), a phase 2 study of add-on navtemadlin in patients with a suboptimal response to ruxolitinib.

FIG. 15 illustrates the spleen volume reduction after add-on navtemadlin to a stable dose of ruxolitinib for evaluable subjects with spleen volume assessments at baseline and Week 24. Baseline spleen volume magnetic resonance imaging/computed tomography scans were taken while subjects were on a stable dose of ruxolitinib for ≥8 weeks (e.g., no ruxolitinib wash-out). FIG. 16 illustrates the total symptom score reduction after add-on navtemadlin to a stable dose of ruxolitinib, with baseline TSS assessments taken while subjects were on the same period of a stable dose of ruxolitinib for ≥8 weeks (e.g., no ruxolitinib wash-out). For both FIGS. 15 and 16, no dose increases of ruxolitinib above the stable baseline dose occurred during the 24-week assessment period. The median duration of ruxolitinib treatment prior to the addition of navtemadlin was 21.6 months (range: 7 to 129). Six subjects discontinued treatment prior to Week 24.

In these analyses, the contribution effect of navtemadlin on SVR and TSS from study KRT-232-109 were modeled onto the pooled suboptimal responder data to establish the add-on benefit of navtemadlin from the perspective of the pre-ruxolitinib baseline. This model permitted an estimate of the proportion of suboptimal responder patients who would achieve an SVR25 or TSS30 from the pre-randomization baseline.

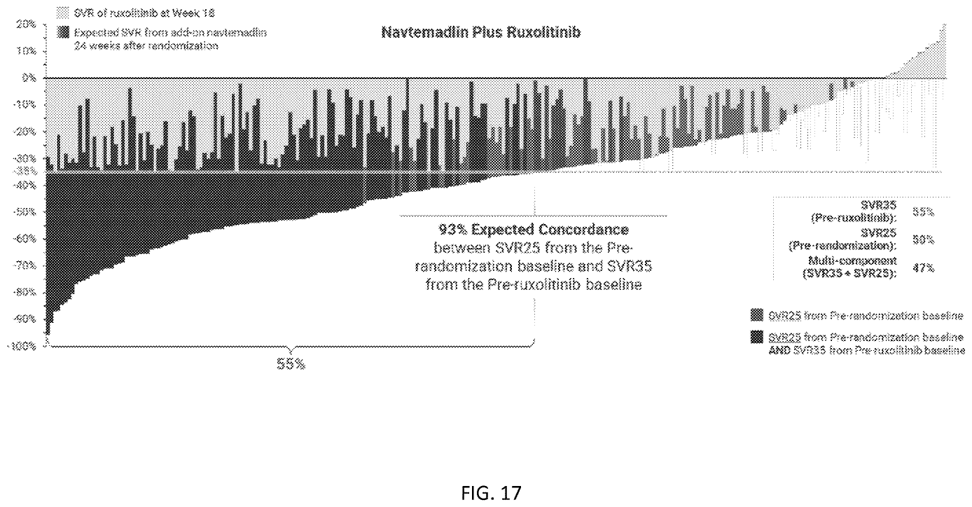

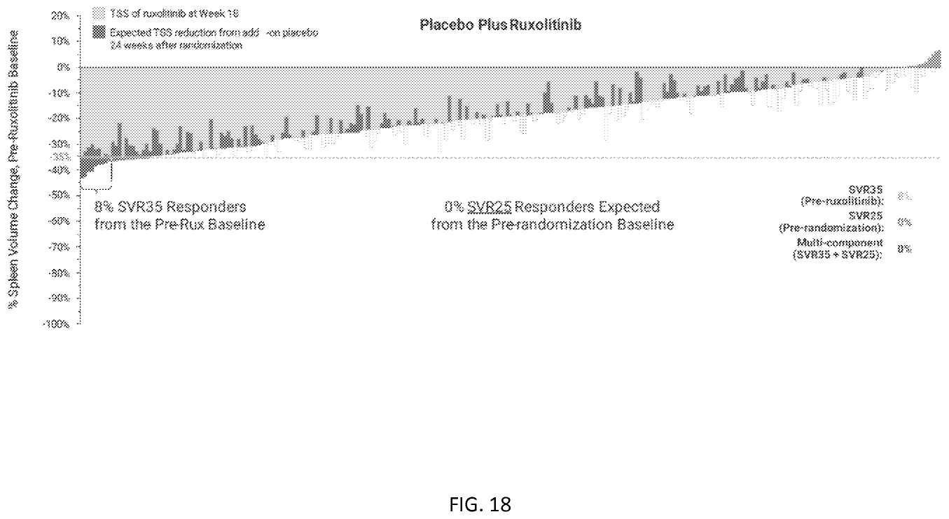

Based on this model, 55% of patients receiving add-on navtemadlin were expected to achieve an SVR35 from the pre-ruxolitinib baseline and 50% an SVR25 from the pre-randomization baseline compared to 8% and 0% for patients receiving add-on placebo, respectively (FIG. 17, FIG. 18). FIG. 17 illustrates the expected SVR for add-on navtemadlin to ruxolitinib, with spleen volume reductions modeled from KRT-232-109 and published phase 3 myelofibrosis studies. Light gray bars depict the baseline distribution of SVR based on pooled, extractable data from published phase 3 JAKi-naïve MF studies for patients with suboptimal spleen response to ruxolitinib monotherapy (±placebo); COMFORT-1, SIMPLIFY-1, and TRANSFORM-1 (Verstovsek 2012; Mesa 2017; Pemmaraju 2023). Blue bars, regardless of shade, show the expected additional benefit of treating patients with add-on navtemadlin. Darkest blue bars depict SVR25 responders from a pre-randomization baseline that also cross the SVR35 threshold from a pre-ruxolitinib baseline (e.g., multi-component SVR endpoint). FIG. 18 illustrates the expected SVR for add-on placebo to ruxolitinib. Dark gray bars show the expected additional benefit of treating patients with add-on placebo. No patients (0% of patients) were modeled to achieve the multi-component SVR endpoint of SVR25 from a pre-randomization baseline AND SVR35 from a pre-ruxolitinib baseline.

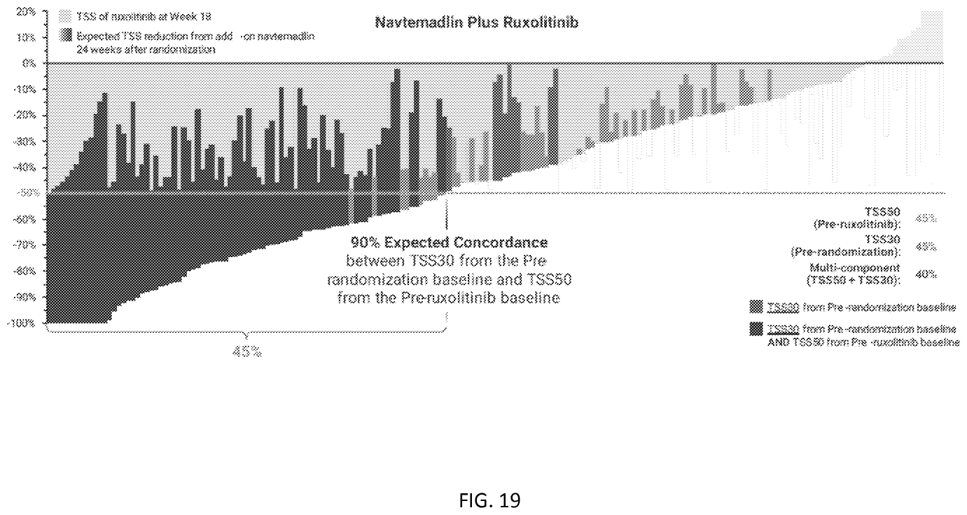

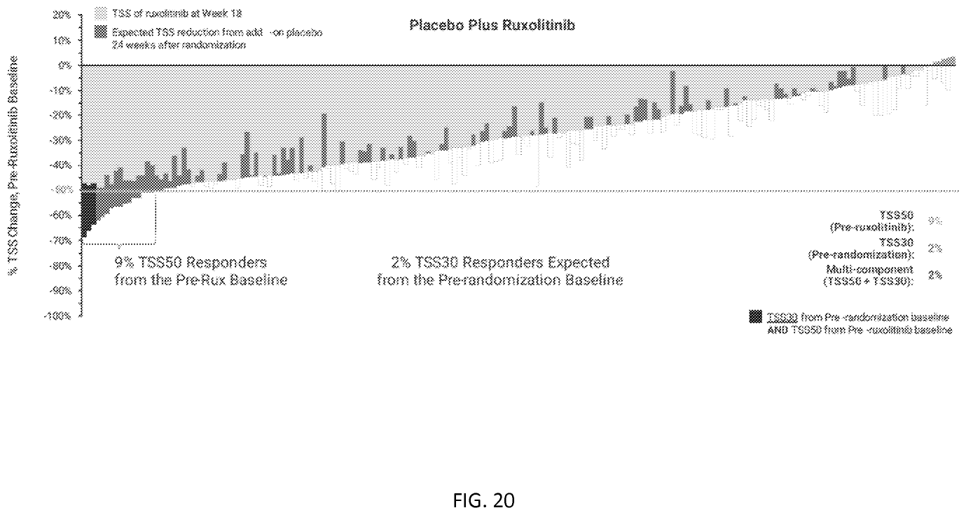

Similarly, 45% of patients receiving add-on navtemadlin were expected to achieve a TSS50 from the pre-ruxolitinib baseline and 45% a TSS30 from the pre-randomization baseline compared to 9% and 2% for add-on placebo (FIG. 19, FIG. 20).

FIG. 19 illustrates the expected TSS reduction add-on navtemadlin to ruxolitinib. Light gray bars depict the baseline distribution of TSS reductions based on pooled extractable data from published phase 3 JAKi-naïve MF studies for patients with suboptimal symptom response to ruxolitinib monotherapy (±placebo). Green bars, regardless of shade, show the expected additional symptom improvement benefit of treating patients with add-on navtemadlin. Darkest green bars depict TSS30 responders from a pre-randomization baseline that also cross the TSS50 threshold from a pre-ruxolitinib baseline (multi-component TSS endpoint). FIG. 20 illustrates the expected TSS reductions for placebo add-on to ruxolitinib. Light gray bars depict the baseline distribution of TSS reductions based on pooled extractable data from published phase 3 JAKi-naïve MF studies for patients with suboptimal symptom response to ruxolitinib monotherapy (±placebo). Darker gray bars show the expected additional benefit of treating patients with add-on placebo. Black bars depict TSS30 responders from a pre-randomization baseline that also cross the TSS50 threshold from a pre-ruxolitinib baseline (e.g., multi-component TSS endpoint).

A 93% concordance was observed between the expected SVR25 from the pre-randomization baseline and the expected SVR35 from the pre-ruxolitinib baseline (e.g., 93% of SVR25 responders would also achieve SVR35 from the pre-ruxolitinib baseline). Similarly, a 90% concordance was observed between the expected TSS30 from the pre-randomization baseline and the expected TSS50 from the pre-ruxolitinib baseline (e.g., 90% of TSS30 responders from would also achieve a TSS50 from the pre-ruxolitinib baseline).

### REFERENCES

Al-Ali H K, Vannucchi A M. Managing patients with myelofibrosis and low platelet counts. Ann Hematol. April 2017; 96(4):537-548.
Aubrey B J, Kelly G L, Janic A, et al. How does p53 induce apoptosis and how does this relate to p53-mediated tumour suppression?Cell Death Differ. January 2018; 25(1):104-113.
Barosi G, Mesa R A, Thiele J, et al. Proposed criteria for the diagnosis of post-polycythemia vera and post-essential thrombocythemia myelofibrosis: a consensus statement from the International Working Group for Myelofibrosis Research and Treatment. Leukemia. February 2008; 22(2):437-8.
Canon J, Osgood T, Olson S H, et al. The MDM2 inhibitor AMG 232 demonstrates robust antitumor efficacy and potentiates the activity of p53-inducing cytotoxic agents. Mol Cancer Ther. March 2015; 14(3):649-58.
Cervantes F, Dupriez B, Pereira A, et al. New prognostic scoring system for primary myelofibrosis based on a study of the International Working Group for Myelofibrosis Research and Treatment. Blood. Mar. 26, 2009; 113(13):2895-901.
Clevenger T, Cheung J, Krantz F, et al. Adding Navtemadlin (nvtm) to Ruxolitinib (rux) Potentiates Apoptosis in Myeloblasts from Patients (pts) with Myelofibrosis (MF). presented at: EHA2023 Congress; 2023; Frankfurt, Germany.
Erba H P, Becker P S, Shami P J, et al. Phase 1b study of the MDM2 inhibitor AMG 232 with or without trametinib in relapsed/refractory acute myeloid leukemia. Blood Adv. Jul. 9, 2019; 3(13):1939-1949.
Fiedler W, Henke R P, Ergun S, et al. Derivation of a new hematopoietic cell line with endothelial features from a patient with transformed myeloproliferative syndrome: a case report. Cancer. Jan. 15, 2000; 88(2):344-51.
Food and Drug Administration. Guidance for Industry Drug-Induced Liver Injury: Premarketing Clinical Evaluation. 2009.
Greenfield G, McPherson S, Mills K, et al. The ruxolitinib effect: understanding how molecular pathogenesis and epigenetic dysregulation impact therapeutic efficacy in myeloproliferative neoplasms. J Transl Med. Dec. 17, 2018; 16(1):360.
Guy W. ECDEU assessment manual for psychopharmacology. U S Department of Health, Education, and Welfare; 1976.
Gwaltney C, Paty J, Kwitkowski V E, et al. Development of a harmonized patient-reported outcome questionnaire to assess myelofibrosis symptoms in clinical trials. Leuk Res. August 2017; 59:26-31.
Harrison C, Kiladjian J J, Al-Ali H K, et al. JAK inhibition with ruxolitinib versus best available therapy for myelofibrosis. N Engl J Med. Mar. 1, 2012; 366(9):787-98.
Harrison C N, Vannucchi A M, Kiladjian J J, et al. Long-term findings from COMFORT-II, a phase 3 study of ruxolitinib vs best available therapy for myelofibrosis. Leukemia. August 2016; 30(8):1701-7.
Harrison C N, Vannucchi A M, Platzbecker U, et al. Momelotinib versus best available therapy in patients with myelofibrosis previously treated with ruxolitinib (SIMPLIFY 2): a randomised, open-label, phase 3 trial. Lancet Haematol. February 2018; 5(2):e73-e81.
Harutyunyan A, Klampfl T, Cazzola M, et al. p53 lesions in leukemic transformation. N Engl J Med. Feb. 3, 2011; 364(5):488-90.
Hussein K, Van Dyke D L, Tefferi A. Conventional cytogenetics in myelofibrosis: literature review and discussion. Eur J Haematol. May 2009; 82(5):329-38.
Jakafi Prescribing Information. Jakafi® (ruxolitinib) prescribing information. Available on the Internet atjakafi.com/pdf/prescribing-information.pdf
Kvasnicka H M, Thiele J, Bueso-Ramos C E, et al. Long-term effects of ruxolitinib versus best available therapy on bone marrow fibrosis in patients with myelofibrosis. J Hematol Oncol. Mar. 15, 2018; 11(1):42.
Lussana F, Rambaldi A. Inflammation and myeloproliferative neoplasms. J Autoimmun. December 2017; 85:58-63.
Mascarenhas J, Hoffman R, Talpaz M, et al. Pacritinib vs Best Available Therapy, Including Ruxolitinib, in Patients with Myelofibrosis: A Randomized Clinical Trial. JAMA Oncol. May 1, 2018; 4(5):652-659.
Mascarenhas J, Jain T, Otoukesh S, et al. An Open-Label, Global, Phase 1B/2 Study Adding Navtemadlin (NVTM) to Ruxolitinib (RUX) in Patients (PTS) with Primary or Secondary Myelofibrosis (MF) Who Have a Suboptimal Response to RUX. presented at: EHA2023 Congress; 2023; Frankfurt, Germany.
Mesa R A, Gotlib J, Gupta V, et al. Effect of ruxolitinib therapy on myelofibrosis-related symptoms and other patient-reported outcomes in COMFORT-I: a randomized, double-blind, placebo-controlled trial. J Clin Oncol. Apr. 1, 2013; 31(10):1285-92.
Mesa R A, Kiladjian J-J, Catalano J V, et al. SIMPLIFY-1: A Phase III randomized trial of momelotinib versus ruxolitinib in Janus kinase inhibitor-naïve patients with myelofibrosis. Journal of Clinical Oncology. 2017; 35(34):3844-3850.
Nakatake M, Monte-Mor B, Debili N, et al. JAK2(V617F) negatively regulates p53 stabilization by enhancing MDM2 via La expression in myeloproliferative neoplasms. Oncogene. Mar. 8, 2012; 31(10):1323-33.
National Institute of Mental Health (NIMH). Patient global impression of change (PGIC). 2017. Available on the Internet at eprovide.mapi-trust.org/instruments/patient-global-impression-of-change.
Newberry K J, Patel K, Masarova L, et al. Clonal evolution and outcomes in myelofibrosis after ruxolitinib discontinuation. Blood. Aug. 31, 2017; 130(9):1125-1131.
Orvain C, Luque Paz D, Dobo I, et al. Circulating CD34⁺ cell count differentiates primary myelofibrosis from other Philadelphia-negative myeloproliferative neoplasms: a pragmatic study. Ann Hematol. October 2016; 95(11):1819-23.
Palandri F, Breccia M, Bonifacio M, et al. Life after ruxolitinib: Reasons for discontinuation, impact of disease phase, and outcomes in 218 patients with myelofibrosis. Cancer. Mar. 15, 2020; 126(6):1243-1252.
Pardanani A, Harrison C, Cortes J E, et al. Safety and Efficacy of Fedratinib in Patients with Primary or Secondary Myelofibrosis: A Randomized Clinical Trial. JAMA Oncol. August 2015; 1(5):643-51.
Pemmaraju N, Mead A J, Somervaille T C, et al. Transform-1: A randomized, double-blind, placebo-controlled, multicenter, international phase 3 study of navitoclax in combination with ruxolitinib versus ruxolitinib plus placebo in patients with untreated myelofibrosis. Blood. 2023 Nov. 28; 142:620.
Pikman Y, Lee B H, Mercher T, et al. MPLW515L is a novel somatic activating mutation in myelofibrosis with myeloid metaplasia. PLoS Med. July 2006; 3(7):e270.
Rampal R K, Grosicki S, Chraniuk D, et al. Pelabresib in Combination with Ruxolitinib for Janus Kinase Inhibitor Treatment-Naïve Patients with Myelofibrosis: Results of the MANIFEST-2 Randomized, Double-Blind, Phase 3 Study. Blood. 2023; 142(1):628.
Raza S, Viswanatha D, Frederick L, et al. TP53 mutations and polymorphisms in primary myelofibrosis. Am J Hematol. February 2012; 87(2):204-6.
Sun D, Li Z, Rew Y, et al. Discovery of AMG 232, a potent, selective, and orally bioavailable MDM2-p53 inhibitor in clinical development. J Med Chem. Feb. 27, 2014; 57(4):1454-72.
Tefferi A, Cervantes F, Mesa R, et al. Revised response criteria for myelofibrosis: International Working Group-Myeloproliferative Neoplasms Research and Treatment (IWG-MRT) and European LeukemiaNet (ELN) consensus report. Blood. Aug. 22, 2013; 122(8):1395-8.
Tefferi A, Thiele J, Orazi A, et al. Proposals and rationale for revision of the World Health Organization diagnostic criteria for polycythemia vera, essential thrombocythemia, and primary myelofibrosis: recommendations from an ad hoc international expert panel. Blood. Aug. 15, 2007; 110(4):1092-7.
Thiele J, Kvasnicka H M, Facchetti F, et al. European consensus on grading bone marrow fibrosis and assessment of cellularity. Haematologica. August 2005; 90(8):1128-32.
Vachhani P, Lange A, Delgado R G, et al. Potential Disease-Modifying Activity of Navtemadlin (KRT-232), a First-in-Class MDM2 Inhibitor, Correlates with Clinical Benefits in Relapsed/Refractory Myelofibrosis (MF). Blood. 2021; 138:3581-3584.
Vachhani P, Perkins A, Mascarenhas J, et al. Disease-modifying activity of navtemadlin correlated with survival outcomes in Janus kinase inhibitor (JAKi) relapsed or refractory myelofibrosis patients. presented at: European Hematology Association; 2023; Frankfurt.
Vannucchi A M, Kantarjian H M, Kiladjian J J, et al. A pooled analysis of overall survival in COMFORT-I and COMFORT-II, 2 randomized phase III trials of ruxolitinib for the treatment of myelofibrosis. Haematologica. September 2015; 100(9):1139-45.
Vaseva A V, Moll U M. The mitochondrial p53 pathway. Biochim Biophys Acta. May 2009; 1787(5):414-20.
Verstovsek S, Mesa R A, Gotlib J, et al. Long-term treatment with ruxolitinib for patients with myelofibrosis: 5-year update from the randomized, double-blind, placebo-controlled, phase 3 COMFORT-I trial. J Hematol Oncol. Feb. 22, 2017; 10(1):55.
Verstovsek S, Mesa R A, Gotlib J, et al. A double-blind, placebo-controlled trial of ruxolitinib for myelofibrosis. N Engl J Med. Mar. 1, 2012; 366(9):799-807.
Vousden K H, Lane Dp. p53 in health and disease. Nat Rev Mol Cell Biol. April 2007; 8(4):275-83.

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown in any combination of FIGS. 1A-C or 2A-C. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A method for treating myelofibrosis in a human subject who has been previously treated for myelofibrosis with ruxolitinib monotherapy and had a suboptimal response to said previous treatment with ruxolitinib monotherapy comprising concurrent administration to the human of a therapeutically effective amount of navtemadlin or a pharmaceutically acceptable salt thereof and ruxolitinib.

2. The method of claim 1, wherein the spleen volume reduction (SVR) in the human after the previous treatment with ruxolitinib monotherapy is <35% compared to the spleen volume prior to the previous treatment with ruxolitinib monotherapy; and the total symptom score (TSS) of the human after the previous treatment with ruxolitinib monotherapy is less than 50% compared to a TSS prior to the previous treatment with ruxolitinib monotherapy.

3. The method of claim 1, wherein the spleen volume reduction (SVR) in the human after the previous treatment with ruxolitinib monotherapy is >zero but <35% compared to the spleen volume prior to the previous treatment with ruxolitinib monotherapy; and the total symptom score (TSS) of the human after the previous treatment with ruxolitinib monotherapy is >zero but less than 50% compared to a TSS prior to the previous treatment with ruxolitinib monotherapy.

4. The method of claim 1, wherein the spleen volume reduction (SVR) in the human after the previous treatment with ruxolitinib monotherapy is >zero but <20% compared to the spleen volume prior to the previous treatment with ruxolitinib monotherapy; and the total symptom score (TSS) of the human after the previous treatment with ruxolitinib monotherapy is >zero but less than 30% compared to a TSS prior to the previous treatment with ruxolitinib monotherapy.

5. The method of claim 1, wherein the spleen volume reduction (SVR) in the human after the previous treatment with ruxolitinib monotherapy is ≥20% but <35% compared to the spleen volume prior to the previous treatment with ruxolitinib monotherapy; and the total symptom score (TSS) of the human after the previous treatment with ruxolitinib monotherapy is ≥30% but <50% compared to a TSS prior to the previous treatment with ruxolitinib monotherapy.

6. The method of claim 1, wherein the previous treatment with ruxolitinib monotherapy is about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, or about 52 weeks.

7. The method of claim 1, wherein the previous treatment with ruxolitinib monotherapy is at least 12 weeks.

8. The method of claim 1, wherein the previous treatment with ruxolitinib monotherapy is at least 18 weeks.

9. The method of claim 1, wherein the concurrent administration of navtemadlin and ruxolitinib is at least 12 weeks, at least 16 weeks, at least 18 weeks, at least 24 weeks, at least 28 weeks, at least 32 weeks, at least 36 weeks, at least 40 weeks, at least 44 weeks, at least 48 weeks, or at least 52 weeks.

10. The method of claim 1, wherein the concurrent administration of navtemadlin and ruxolitinib is at least 24 weeks.

11. The method of claim 1, wherein navtemadlin or a pharmaceutically acceptable salt thereof is administered at a dose of 240 mg daily.

12. The method of claim 1, wherein navtemadlin or a pharmaceutically acceptable salt thereof is administered on days 1-7 of a 28-day treatment cycle; wherein navtemadlin or a pharmaceutically acceptable salt thereof is not administered on days 8-28 of the 28-day treatment cycle.

13. The method of claim 12, wherein navtemadlin or a pharmaceutically acceptable salt thereof is administered for one or more treatment cycles.

14. The method of claim 12, wherein navtemadlin or a pharmaceutically acceptable salt thereof is administered for one, two, three, four, five, six, seven, eight, nine, or ten treatment cycles.

15. The method of claim 13, wherein the spleen volume reduction (SVR) in the human after the one or more treatment cycles with navtemadlin is at least 25%, 30%, 35%, 40%, 45%, 50%, 55%, 60%, 65%, 70%, 75%, 80%, 85%, 90%, or 95% compared to the spleen volume prior to the one or more treatment cycles.

16. The method of claim 13, wherein the spleen volume reduction (SVR) in the human after the one or more treatment cycles with navtemadlin is at least 35%, 40%, 45%, 50%, 55%, 60%, 65%, 70%, 75%, 80%, 85%, 90%, or 95% compared to the spleen volume prior to the previous treatment with ruxolitinib monotherapy.

17. The method of claim 13, wherein the TSS in the human after the one or more treatment cycles with navtemadlin is reduced by at least 30%, 40%, 45%, 50%, 55%, 60%, 65%, 70%, 75%, 80%, 85%, 90%, or 95% compared to a TSS prior to the one or more treatment cycles.

18. The method of claim 13, wherein the TSS of the human after the one or more treatment cycles with navtemadlin is reduced by at least 50%, 55%, 60%, 65%, 70%, 75%, 80%, 85%, 90%, or 95% compared to a TSS prior to the previous treatment with ruxolitinib monotherapy.

19. The method of claim 14, wherein the SVR in the human after the one or more treatment cycles with navtemadlin is at least 25% compared to the spleen volume prior to the one or more treatment cycles and at least 35% compared to the spleen volume prior to the previous treatment with ruxolitinib monotherapy.

20. The method of claim 15, wherein the TSS of the human after the one or more treatment cycles with navtemadlin is reduced by at least 30% compared to a TSS prior to the one or more treatment cycles and at least 50% compared to the TSS prior to the previous treatment with ruxolitinib monotherapy.

21. The method of claim 15, wherein the SVR in the human after the one or more treatment cycles with navtemadlin is at least 25% compared to the spleen volume prior to the one or more treatment cycles and at least 35% compared to the spleen volume prior to the previous treatment with ruxolitinib monotherapy; wherein the TSS of the human after the one or more treatment cycles is reduced by at least 30% compared to a TSS prior to the one or more treatment cycles and at least 50% compared to the TSS prior to the previous treatment with ruxolitinib monotherapy.

22. The method of claim 1, wherein ruxolitinib is administered at a dose of 5 mg, 10 mg, 15 mg, 20 mg, or 25 mg twice daily during ruxolitinib monotherapy.

23. The method of claim 1, wherein ruxolitinib is administered at a dose of 5 mg, 10 mg, 15 mg, 20 mg, or 25 mg twice daily during concurrent administration.

24. The method of claim 1, wherein the human has a JAK2V617F mutation.

25. The method of claim 1, wherein the human does not have TP53 mutation.

26. The method of claim 1, wherein the human is JAK inhibitor-naïve prior to ruxolitinib monotherapy.
